(19) United States
(12) Patent Application Publication
(10) Pub. No.: US 2022/0122696 A1
(43) Pub. Date: Apr. 21, 2022

(54) SYSTEM AND METHOD FOR ACHIEVING HIGH GENE DATA RESOLUTION USING TRAINING SETS

(71) Applicants: Yanmei Huang, Chestnut Hill, MA (US); Isabel Fernandez Escapa, Brooklyn, NY (US); Katherine Lemon, Houston, TX (US); Floyd E. Dewhirst, Medfield, MA (US)

(72) Inventors: Yanmei Huang, Chestnut Hill, MA (US); Isabel Fernandez Escapa, Brooklyn, NY (US); Katherine Lemon, Houston, TX (US); Floyd E. Dewhirst, Medfield, MA (US)

(21) Appl. No.: 17/311,610

(22) PCT Filed: Sep. 27, 2019
(86) PCT No.: PCT/US2019/053634

§ 371 Date: Jun. 7, 2021

Related U.S. Application Data
(60) Provisional application No. 62/775,997, filed on Dec. 6, 2018.

(51) Int. Cl. G16B 40/20; G16B 10/00; C12N 15/10; G06N 20/00

(52) U.S. Cl. CPC G16B 40/20; G16B 10/00; G06N 20/00; C12N 15/1065; C12N 15/1089

(57) ABSTRACT

Systems, methods, and computer program products for generating an enhanced set of sequences for taxonomical classification are disclosed. In various embodiments, a plurality of reference sequences are received. Each of the plurality of reference sequences corresponds to a taxonomical classification. A label corresponding to at least one of the reference sequences is assigned to each of a plurality of supplemental sequences. Each of the plurality of supplemental sequences and each of the plurality of reference sequences are truncated to a region of interest to thereby generate a truncated set of sequences. Similarity is measured between pairs of truncated sequences in the truncated set of sequences to determine whether the similarity is above a predetermined threshold. An intermediate taxonomical label is assigned to the pair of truncated sequences in the truncated set of sequences when the similarity is above the predetermined threshold to thereby generate an enhanced set of sequences.

### CROSS REFERENCE TO RELATED APPLICATION

This application claims the benefit of U.S. provisional patent application No. 62/775,997, filed on Dec. 6, 2018, which is incorporated herein by reference in its entirety.

### STATEMENT REGARDING FEDERALLY SPONSORED RESEARCH

This invention was made with government support under grant numbers TR001102, GM117174, AI101018, DE016937, and DE024468 awarded by the National Institutes of Health. The government has certain rights in the invention.

### BACKGROUND

Embodiments of the present disclosure generally relate to taxonomic classification of microbiome within the human aerodigestive tract. In particular, the present disclosure describes a process for species-level taxonomic classification using a machine learning classifier coupled with minimum entropy decomposition.

### BRIEF SUMMARY

In various embodiments, a method of generating an enhanced set of genomic sequences for taxonomical classification is provided. A plurality of reference genomic sequences is received. Each of the plurality of reference genomic sequences corresponds to a taxonomical classification. Each of a plurality of supplemental genomic sequences is assigned a label corresponding to at least one of the reference genomic sequences. Each of the plurality of supplemental genomic sequences and each of the plurality of reference genomic sequences are truncated to a region of interest to thereby generate a truncated set of genomic sequences. Similarity is measured between pairs of truncated genomic sequences in the truncated set of genomic sequences to determine whether the similarity is above a predetermined threshold. An intermediate taxonomical label is assigned to the pair of truncated genomic sequences in the truncated set of genomic sequences when the similarity is above the predetermined threshold to thereby generate an enhanced set of genomic data.

In various embodiments, a computer program product for generating an enhanced set of genomic sequences for taxonomical classification is provided. The computer program product includes a computer readable storage medium having program instructions embodied therewith. The program instructions are executable by a processor to cause the processor to perform a method including receiving a plurality of reference genomic sequences. Each of the plurality of reference genomic sequences corresponds to a taxonomical classification. Each of a plurality of supplemental genomic sequences is assigned a label corresponding to at least one of the reference genomic sequences. Each of the plurality of supplemental genomic sequences and each of the plurality of reference genomic sequences are truncated to a region of interest to thereby generate a truncated set of genomic sequences. Similarity is measured between pairs of truncated genomic sequences in the truncated set of genomic sequences to determine whether the similarity is above a predetermined threshold. An intermediate taxonomical label is assigned to the pair of truncated genomic sequences in the truncated set of genomic sequences when the similarity is above the predetermined threshold to thereby generate an enhanced set of genomic data.

In various embodiments, a method for generating a species-labelled set of genomic sequences for taxonomical classification is provided. Genomic material is isolated from a microbial source. A predetermined region of the genomic material is amplified to generate a sequence library. The sequence library is sequenced to generate a plurality of genomic sequences. A species is determined for each of the plurality of genomic sequences to thereby generate a species-labelled set of genomic sequences. In various embodiments, determining a species for each of the plurality of genomic sequences includes receiving a plurality of reference genomic sequences. Each of the plurality of reference genomic sequences corresponds to a taxonomical classification. Each of a plurality of supplemental genomic sequences is assigned a label corresponding to at least one of the reference genomic sequences. Each of the plurality of supplemental genomic sequences and each of the plurality of reference genomic sequences are truncated to a region of interest to thereby generate a truncated set of genomic sequences. Similarity is measured between pairs of truncated genomic sequences in the truncated set of genomic sequences to determine whether the similarity is above a predetermined threshold. An intermediate taxonomical label is assigned to the pair of truncated genomic sequences in the truncated set of genomic sequences when the similarity is above the predetermined threshold to thereby generate an enhanced set of genomic data.

### BRIEF DESCRIPTION OF THE DRAWINGS

FIGS. 1A-1D illustrate a process for identifying Human Microbial Taxa (HMTs) from the aerodigestive tract to generate the eHOMD according to embodiments of the present disclosure.

FIGS. 2A-2D illustrate graphs of genera and species in the HMP nares V1-V3 dataset at both an overall and individual level according to embodiments of the present disclosure.

FIGS. 3A-3D illustrate graphs of three common nasal species/supraspecies exhibiting increased differential relative abundance when S. aureus is absent from the nostril microbiome according to embodiments of the present disclosure.

FIG. 4 illustrates a method for sequencing and bioinformatics according to embodiments of the present disclosure.

FIG. 5A illustrates exemplary rRNA gene positions according to embodiments of the present disclosure. FIG. 5B illustrates exemplary rRNA gene length variability according to embodiments of the present disclosure. FIGS. 5C and 5D illustrate exemplary read lengths from a primer according to embodiments of the present disclosure.

FIGS. 6A-6C illustrate exemplary sequencing reads according to embodiments of the present disclosure.

FIG. 7 illustrates a comparison of an OTU workflow and an eHOMD workflow according to embodiments of the present disclosure.

FIGS. 8A-8C illustrate various sequences according to embodiments of the present disclosure.

FIG. 9 illustrates a taxonomy assignment of various sequences according to embodiments of the present disclosure.

FIG. 10A illustrates a graph of reads misclassified according to embodiments of the present disclosure. FIG. 10B illustrates a graph of reads meeting a bootstrap threshold according to embodiments of the present disclosure.

FIG. 11A illustrates a graph of reads misclassified when using V1V3 instead of full length sequences according to embodiments of the present disclosure. FIG. 11B illustrates a graph of reads meeting a bootstrap threshold according to embodiments of the present disclosure.

FIG. 11C illustrates a graph of reads misclassified and reads not called according to embodiments of the present disclosure.

FIG. 12A illustrates a graph of reads meeting a bootstrap threshold according to embodiments of the present disclosure. FIG. 12B illustrates a graph of reads misclassified according to embodiments of the present disclosure.

FIGS. 13A-13E illustrates various clusters of sequences according to embodiments of the present disclosure.

FIG. 14A illustrates a graph of reads meeting a bootstrap threshold according to embodiments of the present disclosure. FIG. 14B illustrates a graph of reads misclassified according to embodiments of the present disclosure.

FIG. 15 illustrates a method for species-level rRNA analysis according to embodiments of the present disclosure.

FIG. 16 illustrates a method for species-level rRNA analysis according to embodiments of the present disclosure.

FIGS. 17A-17B illustrate exemplary graphs of the percentage of 16S rRNA gene sequences identified via blastn for the HMP nares V1-V3 rRNA dataset according to embodiments of the present disclosure.

FIG. 18 depicts an exemplary computing node according to various embodiments of the present disclosure.

### DETAILED DESCRIPTION OF THE PRESENT DISCLOSURE

The human aerodigestive tract, which includes the oral cavity, pharynx, esophagus, nasal passages and sinuses, commonly harbors both harmless and pathogenic bacterial species of the same genus. Optimizing the clinical relevance of microbiome studies for body sites within the aerodigestive tract requires sequence identification at the species or, at least, subgenus level. Understanding the composition and function of the microbiome of the aerodigestive tract is important for understanding human health and disease since aerodigestive tract sites are often colonized by common bacterial pathogens and are associated with prevalent diseases characterized by dysbiosis.

The reductions in the cost of NextGeneration DNA Sequencing (NGS) combined with the increasing ease of determining bacterial community composition using short NGS-generated 16S rRNA gene fragments now make this a practical approach for large-scale molecular epidemiological, clinical and translational studies. 16S ribosomal RNA (or 16S rRNA) is the component of the 30S small subunit of a prokaryotic ribosome that binds to the Shine-Dalgarno sequence. The genes coding for it are referred to as 16S rRNA gene and are used in reconstructing phylogenies, due to the slow rates of evolution of this region of the gene. Optimal clinical relevance of such studies requires at least species-level identification; however, to date, 16S rRNA gene-tag studies of the human microbiome are overwhelmingly limited to genus-level resolution. For example, many studies of nasal microbiota fail to distinguish medically important pathogens, e.g., Staphylococcus aureus, from generally harmless members of the same genus, e.g., Staphylococcus epidermidis. For many bacterial taxa, newer computational methods, e.g., Minimum Entropy Decomposition (MED), an unsupervised form of oligotyping (3), and DADA2 (4), parse NGS-generated short 16S rRNA gene sequences to species-level, sometimes strain-level, resolution. However, to achieve species-level taxonomy assignment for the resulting oligotypes/phylotypes, these methods must be used in conjunction with a high-resolution 16S rRNA gene taxonomic database and a classifying algorithm. Similarly, metagenomic sequencing provides species- and, often, strain-level resolution when coupled with a reference database that includes genomes from multiple strains for each species. For the mouth, the human oral microbiome database (HOMD) has enabled analysis/reanalysis of oral 16S rRNA gene short-fragment datasets with these new computational tools, revealing microbe-microbe and host-microbe species-level relationships, and has been a resource for easy access to genomes from which to build reference sets for metagenomic and metatranscriptomic studies. In the expanded human oral microbiome database (“eHOMD”), the number of genomes linked to aerodigestive tract taxa may be expanded considerably. Thus, the eHOMD may be used as a comprehensive web-based resource enabling the broad community of researchers studying the nasal passages, sinuses, throat, esophagus and mouth to leverage newer high-resolution approaches to study the microbiome of aerodigestive tract body sites in both health and disease. The eHOMD may also serve as an effective resource for lower respiratory tract (LRT) microbiome studies based on the breadth of taxa included, and that many LRT microbes are found in the mouth, pharynx and nasal passages.

In various embodiments, the eHOMD may facilitate rapid comparison of 16S rRNA gene sequences from studies worldwide by providing a systematic provisional naming scheme for unnamed taxa identified through sequencing. In various embodiments, each high-resolution taxon in eHOMD, as defined by 98.5% sequence identity across close-to-full-length 16S rRNA gene sequences, may be assigned a unique Human Microbial Taxon (HMT) number that can be used to search and retrieve that sequence-based taxon from any dataset or database. In various embodiments, this stable provisional taxonomic scheme for unnamed and uncultivated taxa is one of the strengths of eHOMD, since taxon numbers stay the same even when names change.

In various embodiments, the process of generating a revised eHOMD (e.g., eHOMDv15.1) may include using both 16S rRNA gene clone library and short-read datasets. In various embodiments, revised eHOMD new discoveries about the nostril microbiome based on analysis using the eHOMD.

In various embodiments, a system and method for achieving high resolution of genetic data using training sets is described. In various embodiments, these systems and methods relate to sequencing and analysis of genetic information, and in particular to assignment of species-level taxonomy.

In various embodiments, a method to sequence nucleic acids and to generate a high-resolution well curated training set that increases the accuracy for taxonomy assignment using the Ribosomal Database Project (RDP) Classifier is described.

In microbiota studies of most ecosystems and/or habitats, achieving ecologically and/or clinically relevant results requires species-level identification of constituents. Species-level taxonomic assignment is often critically important for host-associated microbial communities because the microbiomes of many eukaryotic hosts include commensal and pathogenic species of the same genus. Also, some microbial genera include species that are site specialists and inhabit distinct niches of a given environment. Metagenomic Whole Genome Sequencing (WGS) promises this for microbiomes for which there are a large number of reference genomes for all of the major species-level constituents.

Currently available reference genome datasets remain incomplete and the cost of metagenomic WGS limits its feasibility to studies of hundreds of samples. In contrast, the low cost of 16S rRNA gene sequencing makes it feasible for studies with hundreds to thousands of samples. But, 16S rRNA gene sequencing studies use read clustering at a percent similarity that constrains resolution to the genus level, i.e., 97% identity.

Recent reviews on best practices and benchmarking for 16S rRNA gene microbiota studies focus on genus-level operational taxonomic unit (OTU) analysis. In various embodiments, for Illumina MySeq, an overlap of the forward and reverse reads may be needed to minimize error rates. In various embodiments, an overlap of the forward and reverse reads may be true for OTU level analysis, but may not be needed if appropriate resolution algorithms are used to parse sequences. In various embodiments, Divisive Amplicon Denoising Algorithm (“DADA2”) and Minimal Entropy Decomposition (“MED”) are two algorithms that may be used to parse 16S rRNA gene short-read sequences to species- or strain-level resolution amplicon sequence variants (ASVs) for DADA2 or oligotypes for MED. There may be no step for assigning taxonomy to oligotypes within MED. In various embodiments, the DADA2 package may include a step to assign genus-level taxonomy to ASVs using the naïve Bayesian RDP Classifier [Wang 2007] followed by species-level assignment using exact string matching.

Microbial databases encompassing broad phylogenetic diversity, such as SILVA, RDP and Greengenes, serve the key role of being applicable to myriad different habitats but this valuable breadth comes with the trade-off of inclusion of taxonomically misannotated 16S rRNA gene sequences. For example, Edgar estimated annotation error rates as high as ˜10-17% in these comprehensive databases. Indeed, SILVA and RDP continue to undergo regular updates and contain a broadly expansive and comprehensive record of 16S rRNA gene sequences from all explored habitats, whereas, Greengenes was last updated in 2013. For habitats that have yet to be deeply interrogated, the access to this breadth outweighs the risk of misclassification due to annotation error. However, once a habitat is sufficiently explored, a habitat-specific database may enable accurate fine-level phylogenetic resolution for taxonomic assignment to ASVs. Existing habitat-specific databases are constructed with different methods and can be used to assign taxonomy via different approaches. Examples of this include the following: 1) stand-alone habitat-specific databases consisting of curated collections of close-to-full-length 16S rRNA gene sequences compiled both from other repositories and by generating new sequences from the habitat of interest, e.g., eHOMD for the human aerodigestive tract, HITdb for the human gut and RIM-DP for rumen; 2) custom addition of compiled sequences from a specific habitat of interest to augment a broad general database, e.g., HBDB for honey bee, DictDB for termite and cockroach gut, SILVA19Rum for rumen and MiDAS for activated sludge; 3) both a general and a habitat-specific database combined in the same pipeline, e.g., a general database followed by a most common ancestors approach with a custom species-level phylogeny of selected human-associated genera with pathogenic members and FreshTrain with the TaxAss workflow for freshwater. Many of these databases may be used to train classifiers for taxonomy assignment.

The naïve Bayesian RDP Classifier is one of several effective algorithms for assigning taxonomy, all of which require a training set. Properly formatted versions of the broad 16S rRNA gene databases (e.g., SILVA, RDP and/or Greengenes) are available to train the most popular implementations of the naïve Bayesian RDP Classifier. The quality of the training set strongly influences taxonomic assignment and habitat-specific training sets have been developed to increase accuracy of taxonomic assignments. However, the resolution of available training sets is mostly limited to the genus level. An exception is the manually curated subset of the Greengenes database corresponding to 89 clinically relevant bacterial genera that was used to assign species-level taxonomy of full-length 16S rRNA gene sequences of clinical isolates. Notwithstanding, species-level taxonomy assignment of short-read 16S rRNA gene datasets remains a challenge.

This disclosure relates to the use of a high-resolution well curated environmentally specific database that generates a high-resolution well curated training set that is leveraging the strength of the naïve Bayesian RDP Classifier to achieve species- or supraspecies (i.e., subgenus)-level taxonomic assignment to ASVs and oligotypes from the microbiomes of the human aerodigestive tract. By using the RDP Classifier, the ASVs or oligotypes are never clustered, and therefore the resolution achieved by DADA2 and MED are maintained in the process of taxonomy assignment.

In one aspect, the method includes 16S rRNA gene region sequencing. The choice of the 16S rRNA gene regions for short-read sequencing places an upper bound on the amount of species-level resolution that is possible within a dataset. Therefore, for any habitat of interest, it is key to determine which regions provide the most information for distinguishing the species that are common to that habitat. For the habitats within the human aerodigestive tract, i.e., nasal passages, more taxa are distinguishable with V1-V3 than with the commonly used V3-V4 region.

In some aspects, highly-informative 16S rRNA gene subregions within V1-V3 have been identified. A Shannon Entropy plot for the entropy across the V1-V3 region by projecting across the aligned region has been generated. Based on the entropy plot, it has been determined that sequencing less than 300 bp (base pairs) from the V1 primer and less than 150 bp (base pairs) from the V3 primer would capture that majority of the sequenced diversity needed to maximize species-level taxonomic assignment to the V1-V3 region for species included in eHOMD. In various embodiments, simulation data may be generated. In various embodiments, starting from the ˜770 eHOMD RefSeqs, a variability may be introduced to generate a simulated full V1-V3 16S rRNA gene dataset consisting of distinct sequences. In various embodiments, multiple versions of this simulated V1-V3 dataset may be created to mimic nonoverlapping paired Illumina sequences from the V1 and V3 primers. In various embodiments, the ˜770 eHOMD RefSeqs may be used as a training set (FL_RefSeqs) for a naïve Bayesian RDP Classifier to determine the percentage of sequences classified to the species-level at different boot strap values for each version of the simulated V1-V3 dataset. In various embodiments, from each primer, V1 and V3, separately, visualization is performed to determine at what read length there was no longer a gain in the percentage of classified sequences. In various embodiments, a read length from primer V1 at 350 bp may be fixed, based on the determination above that the first 300 bp capture the majority of sequence variability and the extra 50 bp allowed for variability in region length. In various embodiments, the read length from primer V3 at 200 bp may be fixed. In various embodiments, the percentage of sequences classified at species level may be determined as the read length from the opposite primer increased. In various embodiments, based on these assays, with 350 bp from V1, species-level assignment plateaued across all bootstrap values at 70 bp from primer V3. In various embodiments, with 200 bp from primer V3, species-level assignment started to plateau across all bootstrap values between 210 and 250. In various embodiments, species-level identification may be achieved for the majority of taxa in eHOMD while allowing for a gap in the V1-V3 region sequence. In various embodiments, these results may establish guidance for actual read lengths needed with Illumina sequencing of the 16S rRNA gene V1-V3 region. In various embodiments, an advantage of the naïve Bayesian RDP Classifier, a k-mer-based Bayesian approach, is that it may tolerate nonoverlapping sequences from the two primers and provide a single taxonomy assignment based on data in Read 1 (R1) and Read 2 (R2). In various embodiments, based on the variability in length of actual V1-V3 regions among taxa, using nonoverlapping sequences from primers V1 and V3 may effectively vary both the size and position of the nonoverlapping area. In various embodiments, the naïve Bayesian RDP Classifier may perform taxonomy assignment of the simulated dataset.

Further included are the methods to obtain high-quality sequences were using primers V1 and V3 with 500 cycles on the Illumina MiSeq. Standard 2×250 sequencing resulted in extremely poor-quality sequences from the primer V1, i.e., Read 2 (R2), failing to achieve 210 to 250 high-quality bpx. In various embodiments, the 16S rRNA gene sequence may be very highly conserved immediately 3′ of primer V1, which can result in clustering errors. In various embodiments, when Read 1 (R1) was from primer V1 this resulted in catastrophic sequencing failure. In various embodiments, the quality may drop off sooner from R2 than from R1 when R2 was from primer V1 sequences were obtained. Amplicon-based libraries may be challenging to sequence using the Illumina 2-channel sequencing chemistry due to, e.g., low diversity, which may hinder correct identification of DNA clusters and accurate base calling. In various embodiments, to mitigate this, Read 1 (R1) may start from the V3 primer instead of V1, since clusters are defined very early in an Illumina run (first 4 cycles) and sequences are mostly identical in the first positions immediately 3′ of the V1 primer. In various embodiments, a significant improvement in sequence quality may be obtained by steps as follows: 1) increasing the proportion of PhiX from 10-15% up to 30-40%, which we postulate minimizes over clustering; and 2) performing asymmetrical sequencing of R1 and R2 with R1=100 nt and R2=400 nt, which we postulate. In various embodiments, after trimming poor quality sequence from each read, high-quality sequences of at least 200 bp from primer V1 and 100 bp from primer V3 may be generated. In various embodiments, 250 bp from primer V1 and 100 bp from primer V3 may be used for the simulated eHOMD dataset (eHOMD V1-V3 250_100) used to test each step in the development of the eHOMD V1-V3 16S rRNA gene Training Set for the naïve Bayesian RDP Classifier.

In some aspects, the accuracy of species-level taxonomic classification is improved by using compilations of closely-related sequences rather than a few RefSeqs for each taxon in the training set. In various embodiments, the naïve Bayesian RDP Classifier may be used to achieve genus-level taxonomy assignment. In various embodiments, taxonomy assignment may be limited by the resolution to which sequences in datasets are parsed and by the nature of the training set used. In various embodiments, these limitations may be overcome using approaches such as oligotyping/MED, DADA2, or zero-radius OTU (ZOTU) to parse sequence variants at high resolution. In various embodiments, limitations inherent in training sets may be overcome in the following ways. In various embodiments, the algorithm of the naïve Bayesian RDP Classifier may indicate that a training set with a larger number of sequences representing each taxon will result in more confident taxonomy assignment. In various embodiments, as shown in equation 1 below, based on the conditional probability for a member of a taxon (7), the higher the number of possible times a given “k-mer” (word or wᵢ) could exist in the training set, the greater the confidence with which assignment of that taxon is made. In various embodiments, as the number of sequences (M) in the training set increases, the number of assignments passing the bootstrap threshold should increase. In various embodiments, based on this, to increase M, eHOMD RefSeqs may be used as bait to capture all the sequences present in NCBI matching to each RefSeq at 99% identity over 99% coverage. In various embodiments, after removing any duplicate sequences, the compilation of sequences for each taxon were then combined into a close-to-full-length (FL) eHOMD Compilation Training Set (FL_Compilation TS). In various embodiments, the simulation dataset consists of sequences with known, i.e., true, taxonomic assignment. In various embodiments, these known classifications allow for assessing the level of misclassification that occurred with different versions of our training set. Using the FL_Compilation TS, the percentage of reads in the eHOMD V1-V3 250_100 simulated dataset that classified at the species-level at incremental bootstrap values from 50 to 100 compared to with our FL_RefSeqs TS was assessed and an increase was observed, except at a bootstrap of 100. In various embodiments, additional reads classified with the FL RefSeq TS at a bootstrap of 100 represent over classification, which may be a problem in training sets with only a few representative sequences for each taxon.

$$P ( {w}_{i} \vert T ) = \frac{m ( {w}_{i} ) + {P}_{i}}{M + 1} \qquad ( Eqn . 1 )$$

In various embodiments, at each bootstrap threshold, the percentage of reads that were misclassified using FL_RefSeqs TS was low. In various embodiments, use of the FL_Compilation TS may result in an >50% decrease in the percentage of misclassified reads. In various embodiments, classification of the simulation dataset may result in a reduced error rate and increased confidence level when using a training set consisting of a compilation of closely related sequences instead of single reference sequences.

In some aspects, closely related taxa is combined into supraspecies to maximize the percentage of reads assigned at a subgenus level. A decrease in the percentage of reads assigned subgenus-level taxonomy using the V1V3_Compilation_Clean TS was observed. Tagging the identical V1-V3 sequences from more than one species with a combined name resulted in more assignment options within groups of closely related species with highly conserved 16S rRNA gene sequences.

In some aspects, short-read sequencing of the most informative 16S rRNA gene regions for the bacteria native to the environment of interest provide the maximal amount of species-level information and minimizes the number of unresolvable species.

In some aspects, a method to generate clusters of existing close-to-full length 16S rRNA gene sequences at the 99% level around highly curated reference sequences to increase the accuracy for taxonomy assignment using the RDP Classifier.

In some aspects, adding an intermediate level of assignment between genus and species, a supraspecies level, to all sequences that belong to 99% clusters that overlap increased the % of sequences assigned taxonomy below the genus level, by preventing the default of difficult-to-assign sequences to the genus level.

The present disclosure describes one or more embodiments, and it should be appreciated that many equivalents, alternatives, variations, and modifications, aside from those expressly stated, are possible and within the scope of the invention.

In various embodiments, the methods described herein may be used to generate a high resolution dataset of genomic sequences for taxonomical classification. In various embodiments, the high resolution dataset may include species labels. In various embodiments, the high resolution dataset may include sub-genus labels. In various embodiments, the methods described herein may allow classification accuracy to increase from 10-50% (using techniques known in the art) to more than 90% with error of 0.5% or less. In various embodiments, the methods described herein allow for the use of shorter sequences without losing resolution of the sequencing operation. In various embodiments, the methods described herein may increase the speed of taxonomical classification of genomic sequences by up to 3 times when compared to methods known in the art. In various embodiments, microbial sources of genomic material may be sampled from one or more locations of a body including: oral, nasal, sinus, esophagus, trout, lower/upper respiratory tract. In various embodiments, the microbial sources may be sampled from healthy and/or diseased individuals. In various embodiments, the methods described herein may be applied, without limitation, to other ecosystems where microbial sources may be sampled (e.g., synthetic surfaces, natural surfaces, plants, animals, bodies of water, earth, etc.).

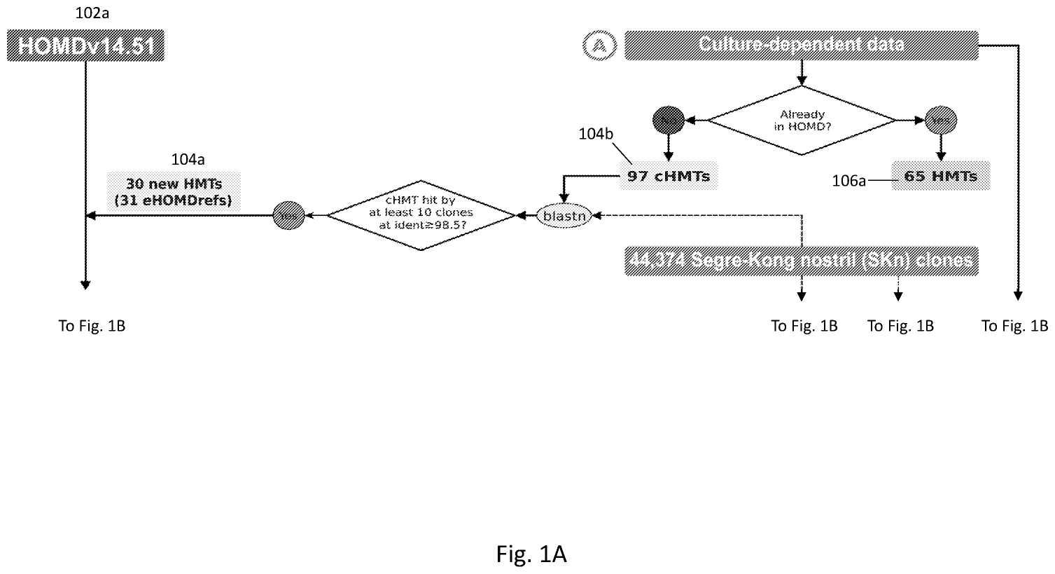

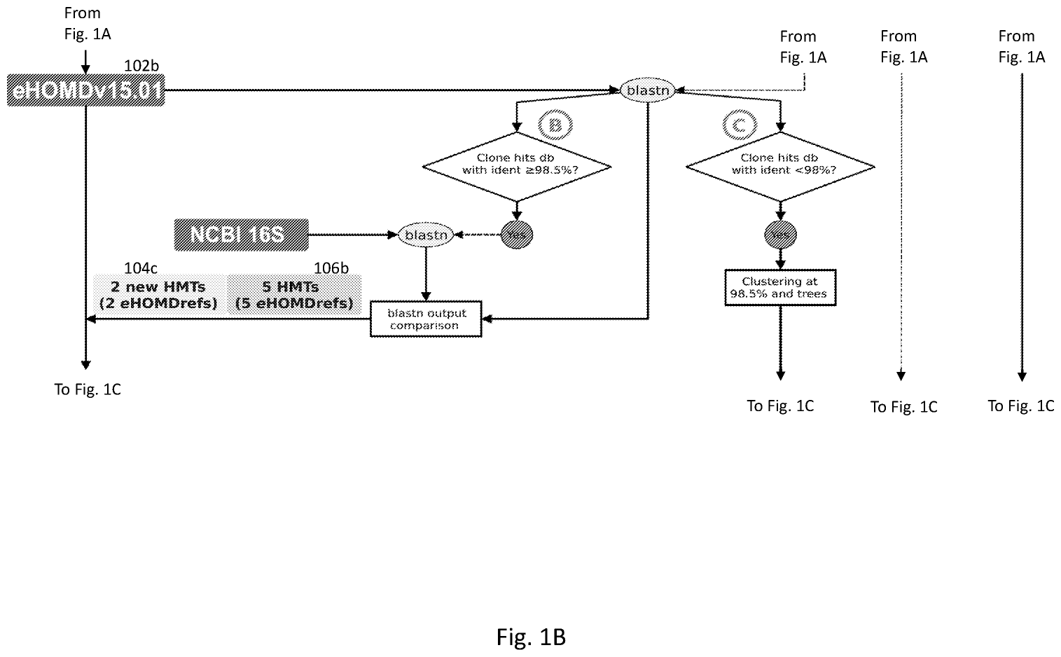

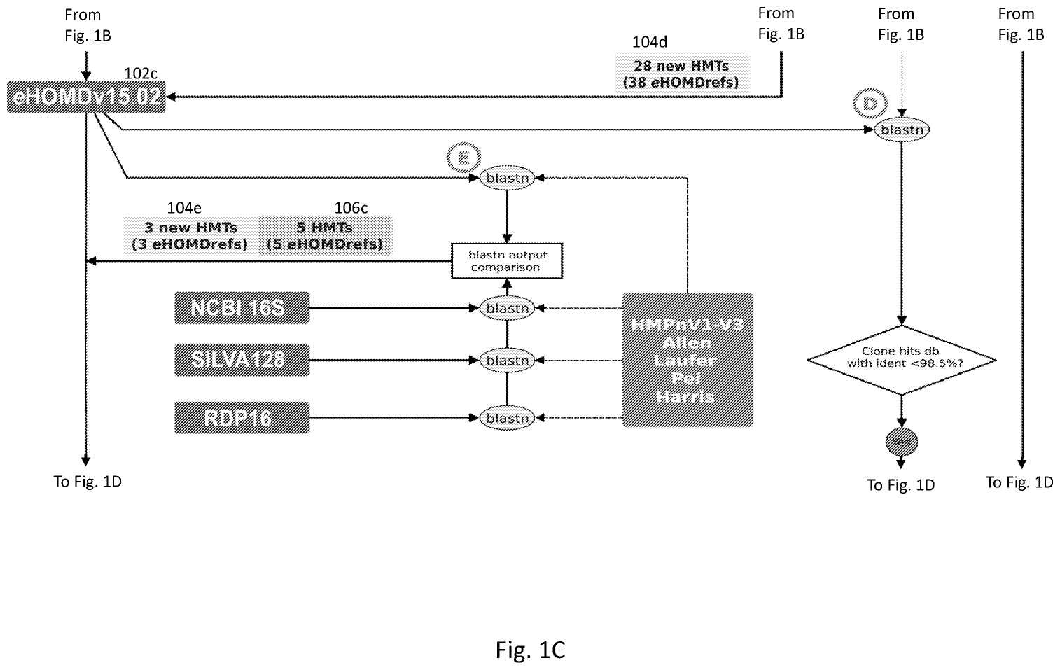

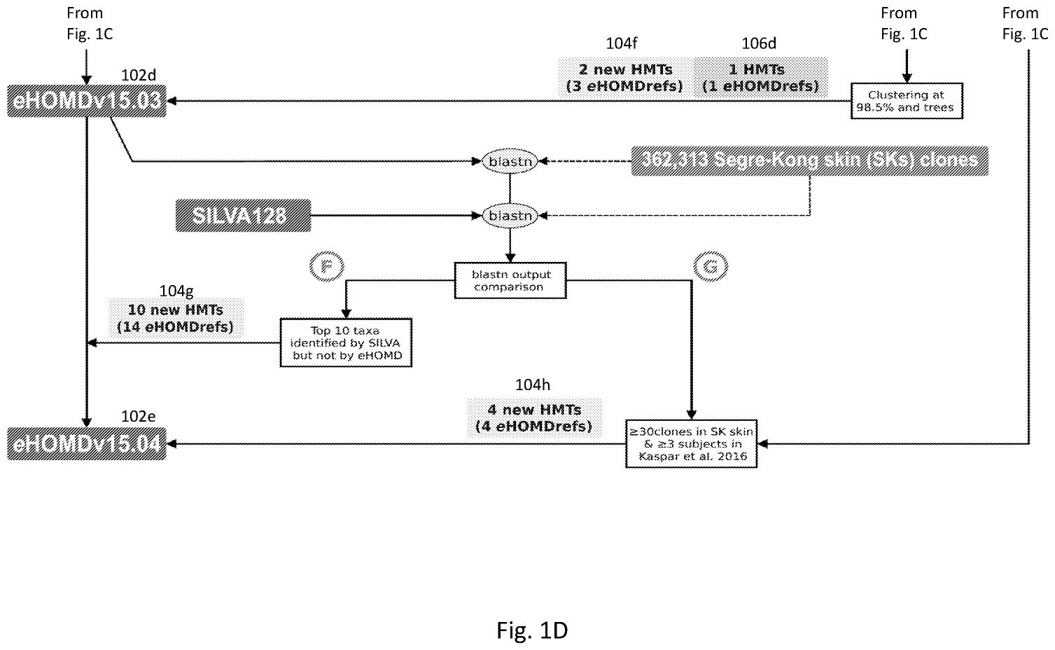

FIGS. 1A-1D illustrate a process for identifying Human Microbial Taxa (HMTs) from the aerodigestive tract to generate the eHOMD according to embodiments of the present disclosure. In various embodiments, the process for identifying Human Microbial Taxa (HMTs) from the aerodigestive tract to generate the eHOMD may be an interative process where the eHOMD is revised each time. In various embodiments, eHOMD database 102a is an prior HOMD taxonomy. In various embodiments, HMT replaces the old HOMD taxonomy prefix HOT (human oral taxon). In various embodiments, eHOMD database 102b is generated by adding bacterial species from culture-dependent studies. In various embodiments, eHOMD database 102c is generated by identifying additional HMTs from a dataset of 16S rRNA gene clones from human nostrils. In various embodiments, eHOMD database 102d is generated by identifying additional candidate taxa from culture-independent studies of aerodigestive tract microbiomes. In various embodiments, eHOMD database 102e is generated by identifying additional candidate taxa from a dataset of 16S rRNA gene clones from human skin. In FIGS. 1A-1D, NCBI 16S represents the NCBI 16 Microbial database, eHOMDref represents the eHOMD reference sequence, db represents the database and ident represents identity. In various embodiments, any suitable microbiome datasets as are known in the art may be used to revise the eHOMD. In various embodiments, the process may include adding new HMTs 104a-104h and/or new eHOMDrefs for present HMTs 106a-106d.

In various embodiments, nucleotide Basic Local Alignment Search Tool (“blastn”) may be use to find regions of local similarity between sequences. In various embodiments, Blastn may search nucleotide databases by using a nucleotide query.

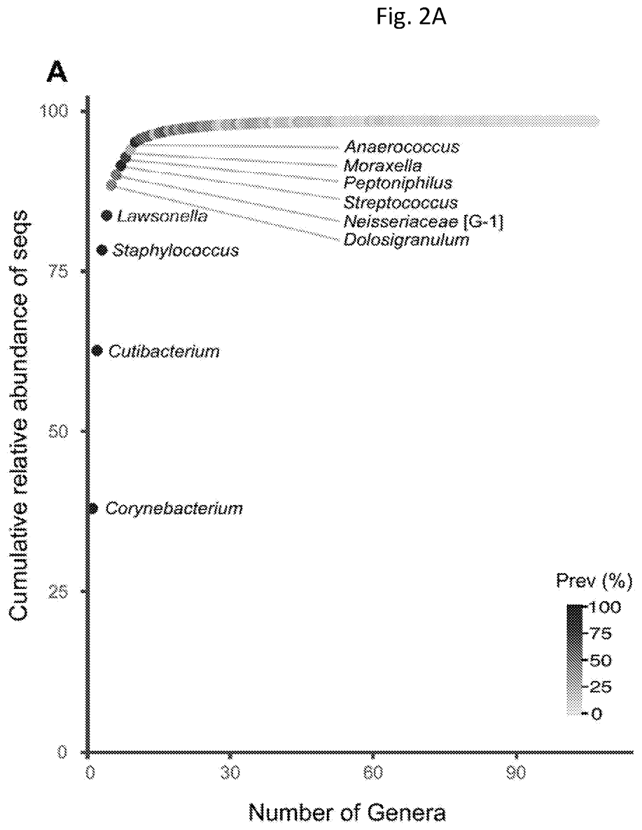

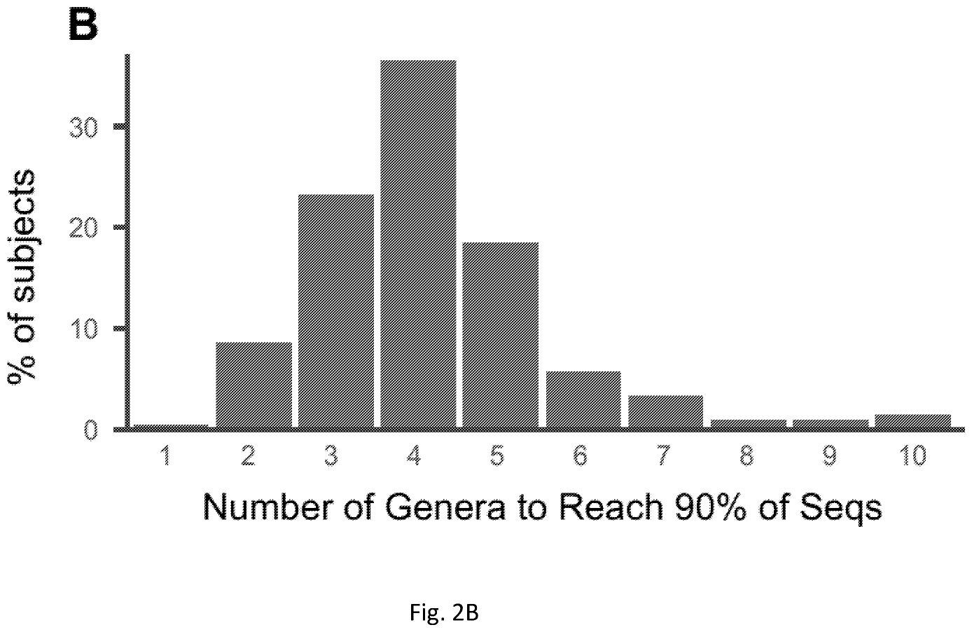

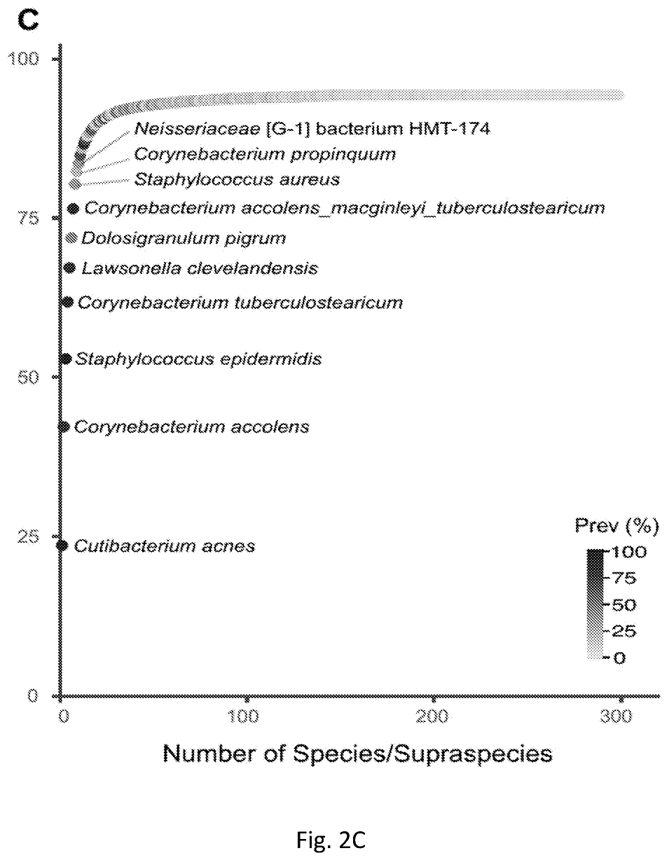

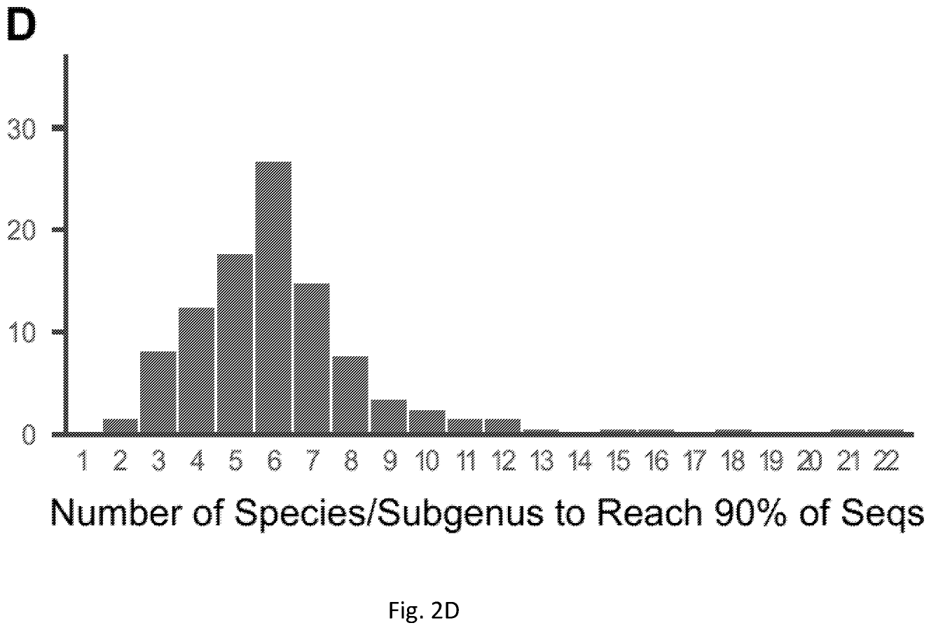

FIGS. 2A-2D illustrate graphs of genera and species in the HMP nares V1-V3 dataset at both an overall and individual level according to embodiments of the present disclosure. FIGS. 2A-2D illustrate that a small number of genera and species account for the majority of taxa in the HMP nares V1-V3 dataset at both an overall and individual level. Taxa identified in the reanalysis of the HMP nostril V1-V3 dataset graphed based on cumulative relative abundance of sequences at the genus- (FIG. 2A) and species/supraspecies- (FIG. 2C) level. The top 10 taxa are labeled. Prevalence (Prev) in % is indicated by the color gradient. The genus Cutibacterium includes species formerly known as the cutaneous Propionibacterium species, e.g., P. acnes (70). The minimum number of taxa at the genus- (FIG. 2B) and species/supraspecies- (FIG. 2D) level that accounted for 90% of the total sequences in each person's sample based on a table of taxa ranked by cumulative abundance from greatest to least. Ten or fewer species/supraspecies accounted for 90% of the sequences in 94% of the 210 HMP participants in this reanalysis. The cumulative relative abundance of sequences does not reach 100% because (1) 1.5% of the reads could not be assigned a genus and (2) 4.9% of the reads could not be assigned a species/supraspecies.

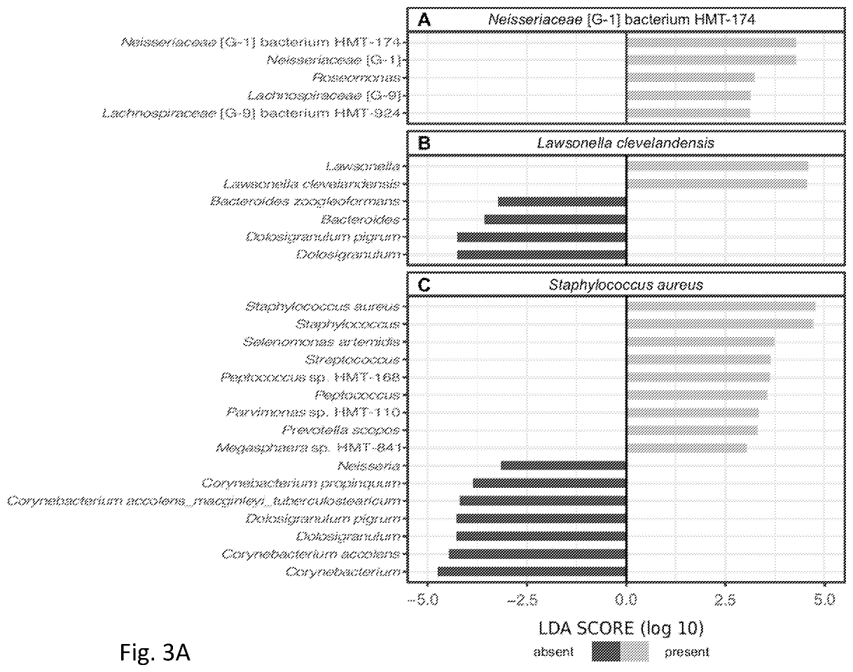

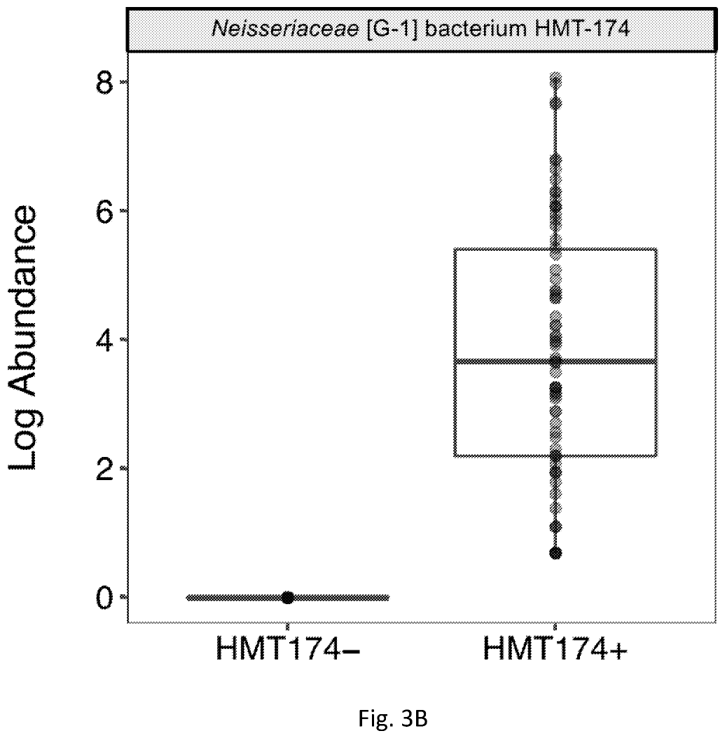

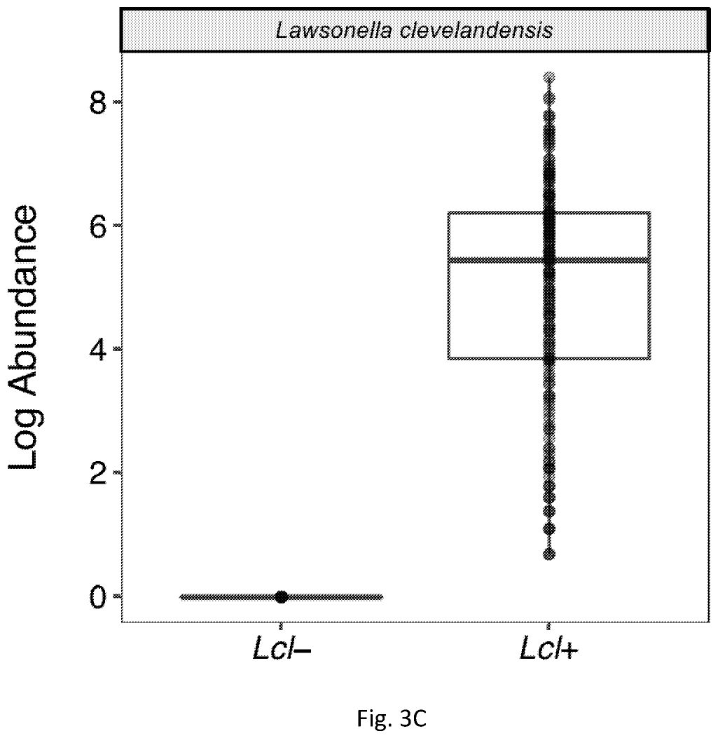

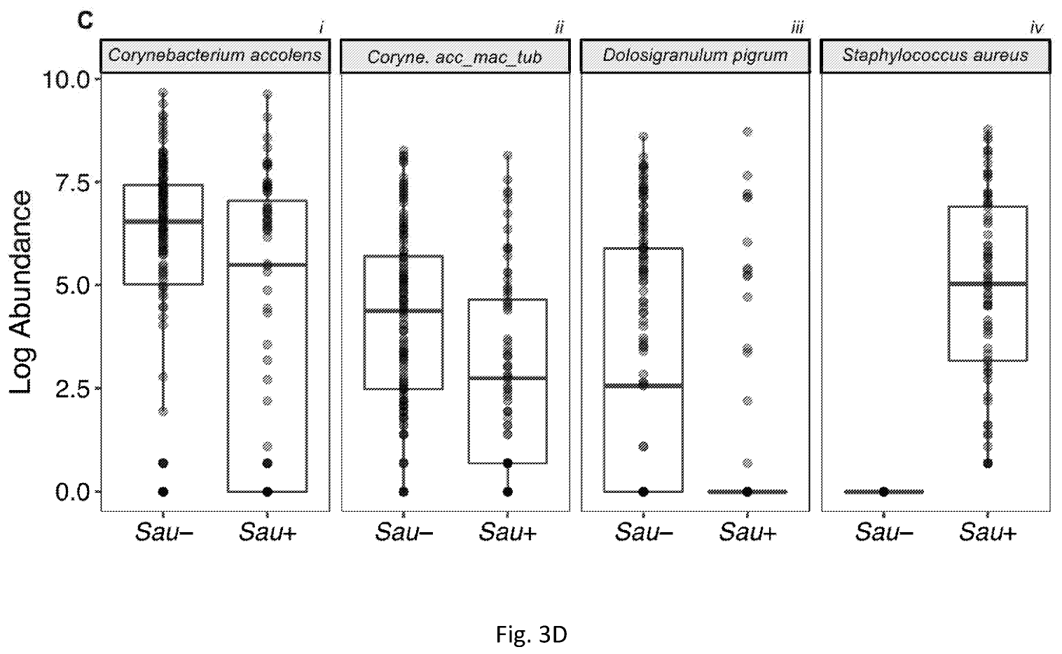

FIGS. 3A-3D illustrates a graph of three common nasal species/supraspecies exhibiting increased differential relative abundance when S. aureus is absent from the nostril microbiome according to embodiments of the present disclosure. In particular, three common nasal species/supraspecies exhibit increased differential relative abundance when S. aureus is absent from the nostril microbiome. In contrast, no other species showed differential abundance based on the presence/absence of Neisseriaceae [G-1] bacterium HMT-174 or Lawsonella clevelandensis. We used ANCOM to analyze species/supraspecies-level composition of the HMP nares V1-V3 dataset when Neisseriaceae [G-1] bacterium HMT-174(FIG. 3B), L. clevelandensis (Lcl) (FIG. 3C), or S. aureus (Sau) (FIG. 3D) were either absent (−) or present (+). Results were corrected for multiple testing. The dark bar represents the median, and lower and upper hinges correspond to the first and third quartiles. Each gray dot represents a sample, and multiple overlapping dots appear black. Coryne. acc_mac_tub represents the supraspecies Corynebacterium accolens_macginleyi_tuberculostearicum.

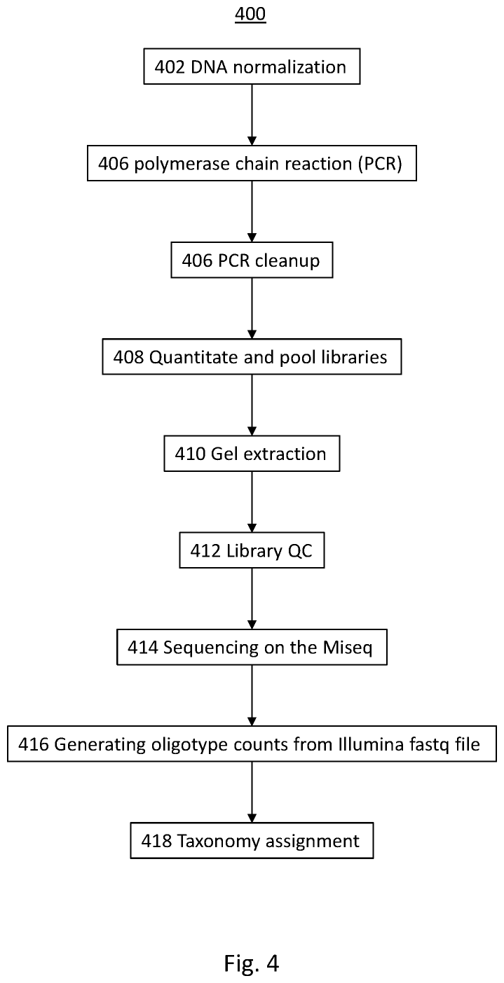

FIG. 4 illustrates a method 400 for sequencing and bioinformatics according to embodiments of the present disclosure. At 402, DNA normalization is performed. In various embodiments, the DNA samples may be normalized to an approximate concentration (e.g., 25 ng/ul) using sterile nuclease free water.

At 404, polymerase chaine reaction (PCR) is performed. In various embodiments, for the PCR reaction, the volumes of DNA template and sterile nuclease free water may vary depending on the DNA concentration. In various embodiments, the combined total of both volumes may equal 28 ul. In various embodiments, a total of 50 ng of the template is added to the following PCR reaction: 10 ul of DNA template, 20 ul of 5 Prime Hot MM, 1 ul of Forward (10 uM), 1 ul of Reverse (10 uM), 18 ul of Nuclease free water. In various embodiments, the reaction total may be 50 ul.

In various embodiments, standard PCR protocols as are known in the art may be used. In various embodiments, the PCR reaction may be run with the following conditions:

| Step | Temp. | Time | |
|---|---|---|---|
| 1 | 94° C. | 3 | min |
| 2 | 94° C. | 45 | sec |
| 3 | 55° C. | 1 min for 30 cycles | |
| 4 | 72° C. | 1.5 | min |
| 5 | 72° C. | 10 | min |

| | | |
|---|---|---|
| 6 | 4° C. | hold |

In various embodiments, primers used for PCR may include primers for the V1V3 region of a gene. In various embodiments, primers used for PCR may include both forward and reverse primers having indices (barcodes). In various embodiments, twelve i7.V1.SA70x (˜27R) primers and eight i5.V3.SA50x (˜518F) primers may correspond to the NexteraXT A indexes. In various embodiments, a ˜518F primer may include the following sequence AATGATACGGCGACCACCGAGATCTACACATCGTACGTATGGTAATTCAATTACCGC GGCTGCTGG where AATGATACGGCGACCACCGAGATCTACAC is a 5′ adaptor, ATCGTACG is a barcode, TATGGTAATT is a primer pad region, and CAATTACCGCGGCTGCTGG is a 16S forward primer. In various embodiments, a ˜27R primer may include the following sequence CAAGCAGAAGACGGCATACGAGATAACTCTCGAGTCAGTCAGCCGAGTTTGATCMT GGCTCAG where CAAGCAGAAGACGGCATACGAGAT is a reverse compliment to a 3′ adaptor, AACTCTCG is a barcode, AGTCAGTCAG is a primer pad region, and CCGAGTTTGATCMTGGCTCAG is a 16S reverse primer.

At 406, PCR cleanup may be performed. In various embodiments, PCR products may be purified using AmpureXP beads using protocols as are known in the art.

At 408, the reaction may be quantitated and libraries may be pooled. In various embodiments, the purified PCR products were quantitated using the nanodrop. Equal amounts of each sample library are pooled into 1 tube (˜100 ng/library).

At 410, Gel extraction may be performed. Typically 80 ul of the pooled library is added to 20 ul of gel loading dye and run on a 1 or 2% agarose gel. The band is cut at ˜590 bps and DNA is extracted using the Qiagen Minelute Gel extraction kit.

At 412, library QC may be performed. In various embodiments, the library may be quantified using a qPCR. In various embodiments, the qPCR may be run on the Roche LIghtcycler using the NEBNext Library Quant Kit for Illumina from NEB. In various embodiments, the samples and standards were prepared and run in triplicate as directed in the protocol, and three dilutions of the library were also run in triplicate.

At 414, sequencing on the Miseq may be performed. In various embodiments, the average concentration determined from the qPCR analysis is used to dilute the purified pooled library to 4 nM. In various embodiments, the sample loading MiSeq Protocol from Illumina was followed for preparation of the library to 10 pM for sequencing. Once the final desired concentration was reached, 20% denatured PhiX was added to the amplicon pool. In various embodiments, the sequencing may be run on a MiSeq using the 500-cycle v2 reagent kit PE kit. In various embodiments, a “Sample Sheet”.csv file was created using Illumina Experiment Manager, and the barcodes used for the samples allows the MiSeq to demultiplex the samples once the run has been completed. In various embodiments, custom sequencing primers may be added to the proper wells of the cartridge. In various embodiments, the sequences of these primers may include:

Read 1 Sequencing Primer (V3_518):
TATGGTAATTCAATTACCGCGGCTGCTGG
Read 2 Sequencing Primer (V1_27):
AGTCAGTCAGCCGAGTTTGATCMTGGCTCAG
Index Sequencing Primer:
CTGAGCCAKGATCAAACTCGGCTGACTGACT

At 416, oligotype counts may be generated from an illumine fastq file. In various embodiments, demultiplexed Fastq files from Illumina sequencing were processed using the DADA2 package (version 1.4.0, Callahan et al., 2016) following a standard workflow of quality trimming, de-replicating, DADA2 denoising, read-pair merging and chimera removal steps with the following parameter settings: For quality trimming, truncLen=c(249, 149), maxEE=c(Inf, Inf), minQ=c(0, 0); for error rate learning and DADA2 denoising, selfConsist=TRUE, pool=TRUE; for chimera removal, method=“pooled”. In various embodiments, the DADA2 program may identify any suitable number of oligotypes from all the samples. For example, the DADA2 program may identify a total of 6436 oligotypes from all the samples, amounting to a total read count of 2,993,794.

At 418, taxonomy assignment may be performed. In various embodiments, the oligotypes identified by the DADA2 program were searched against eHOMD v15.1 16S reference sequences using NCBI BLASTN (Boratyn et al., 2013) with default parameters to identify those that likely originated from species collected in eHOMD, thus can be classified using a Naïve Bayesian Classifier, e.g., the RDP classifier trained with a training dataset. Of the 6436 oligotypes, 1033 were found to match with ≥98% sequence identity and ≥98% sequence coverage to at least one eHOMD reference, accounting for about 72.1% of the total read count. In various embodiments, these oligotypes may be assigned taxonomy using the RDP classifier with acceptable bootstrap value cutoff set at 50. In various embodiments, the remaining oligotypes, i.e., those that do not have good match to any eHOMD reference (5403 out of the 6436 oligotypes, accounting for about 27.1% of the total reads) are assigned taxonomy using a previously described NCBI BLASTN-based pipeline. In various embodiments, 16S rRNA databases used in the BLASTN-based pipeline may include the eHOMD (version 15.1), HOMD 16S rRNA RefSeq Extended Version 1.1 (EXT), GreenGeneGold (GG), and/or the NCBI 16S rRNA reference sequence set. In various embodiments, the number of reference sequences may be 998 (HOMD), 495 (EXT), 3,940 (GG), and 19,670 (NCBI) respectively. In various embodiments, results from the RDP classifier and the BLASTN pipeline may be merged to construct the final taxa count table.

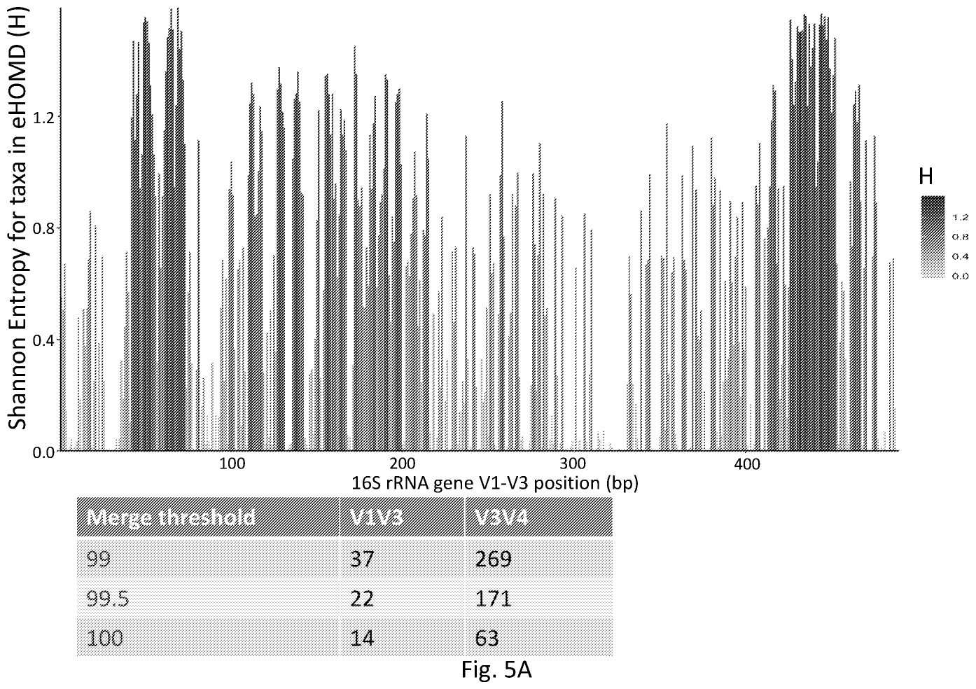

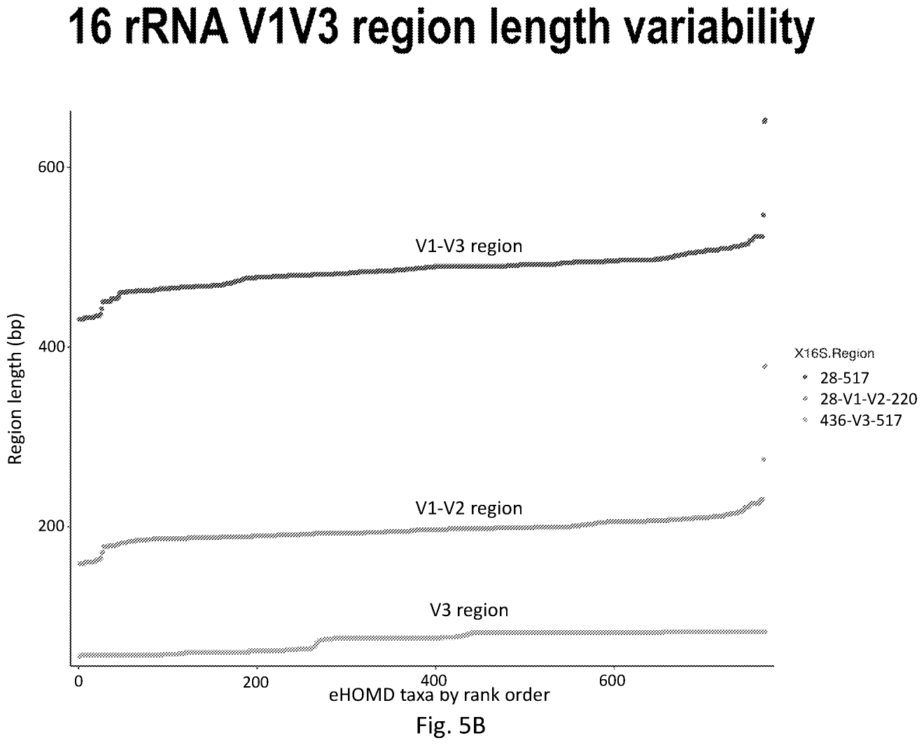

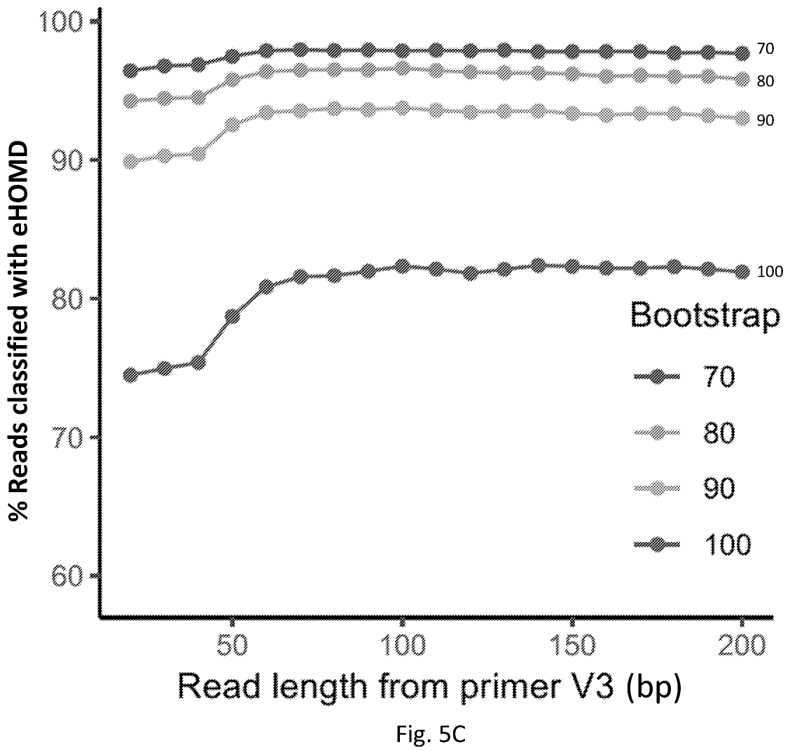

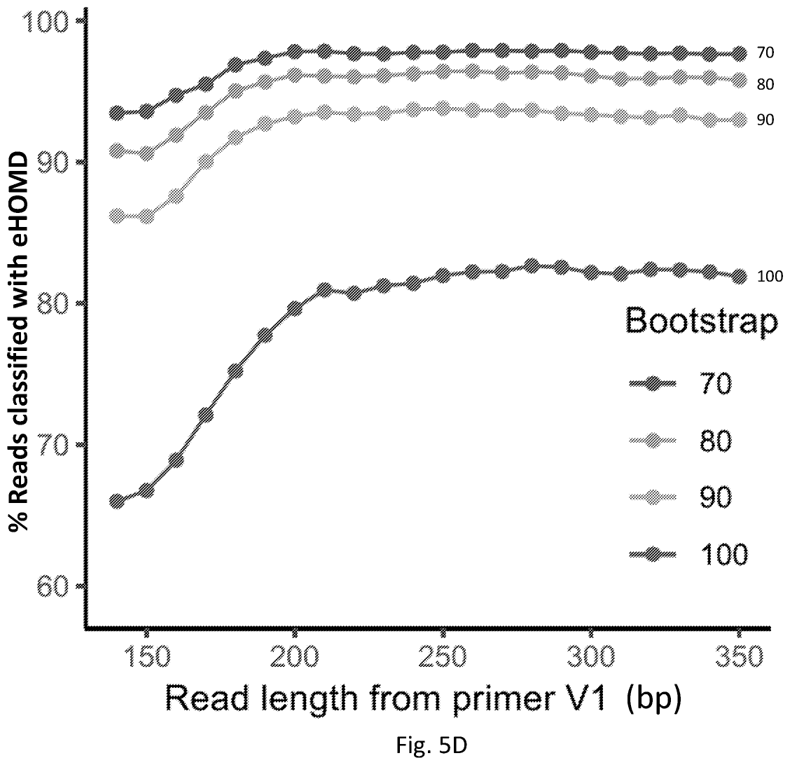

FIG. 5A illustrates exemplary rRNA gene positions according to embodiments of the present disclosure. FIG. 5B illustrates exemplary rRNA gene length variability according to embodiments of the present disclosure. In various embodiments, selecting the V1V2 region may capture more diversity than other regions of the genes, such as, for example, V3V4. FIGS. 5C and 5D illustrate exemplary read lengths from a primer according to embodiments of the present disclosure.

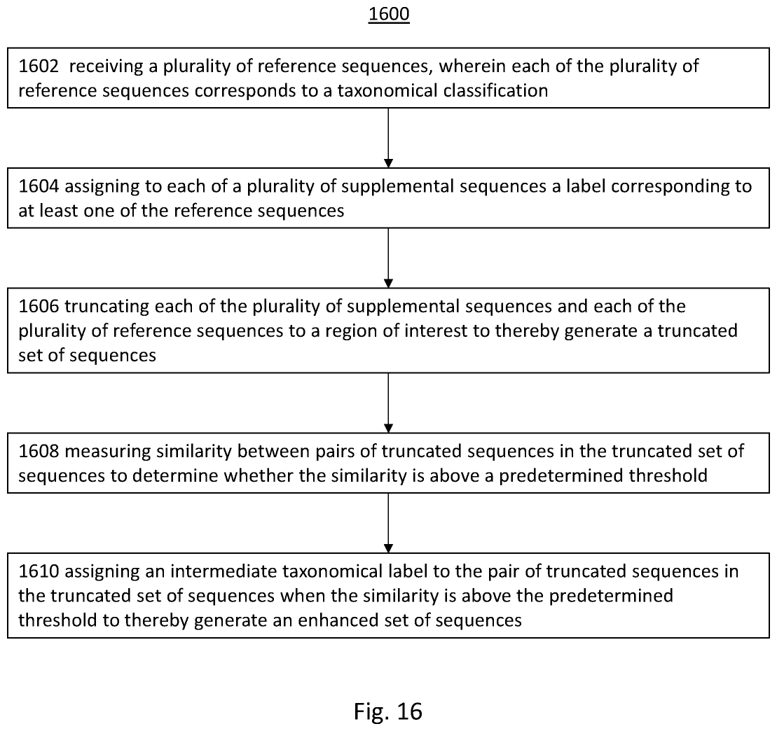

As shown in FIGS. 5A-5D, 16S rRNA gene V1-V3 region sequences do not require overlap to provide maximal information for human aerodigestive tract-associated bacteria. In particular, FIG. 5A shows a rank order of eHOMD taxa based on the nucleotide length of regions V1-V3, V1-V2 and V3 of the 16S rRNA gene. FIG. 5B shows Shannon Entropy (H) across the 16S rRNA gene V1-V3 region for all taxa in eHOMD. For easier visualization, bars are color-coded in gray scale based on their entropy values, i.e., the taller a bar is the darker it is. The percentage of eHOMD-derived simulated reads that can be classified with the FL_eHOMDrefs training set at bootstrap values from 70 to 100 (see key) using a fixed read length of 350 bp from primer V1 and variable read lengths from primer V3 (as shown in FIG. 5C) or a fixed read length of 200 bp from primer V3 and variable read lengths from primer V1 (as shown in FIG. 5D).

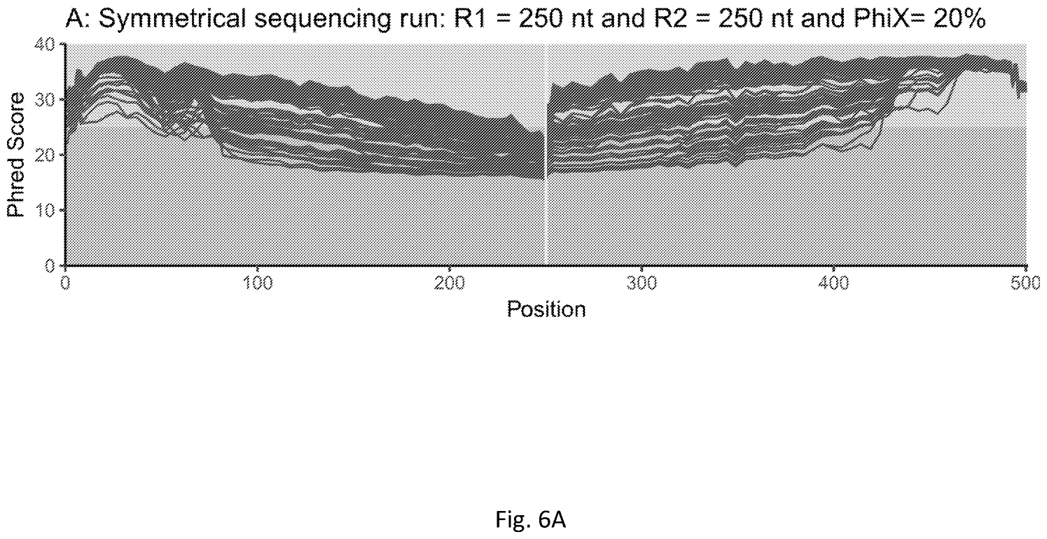

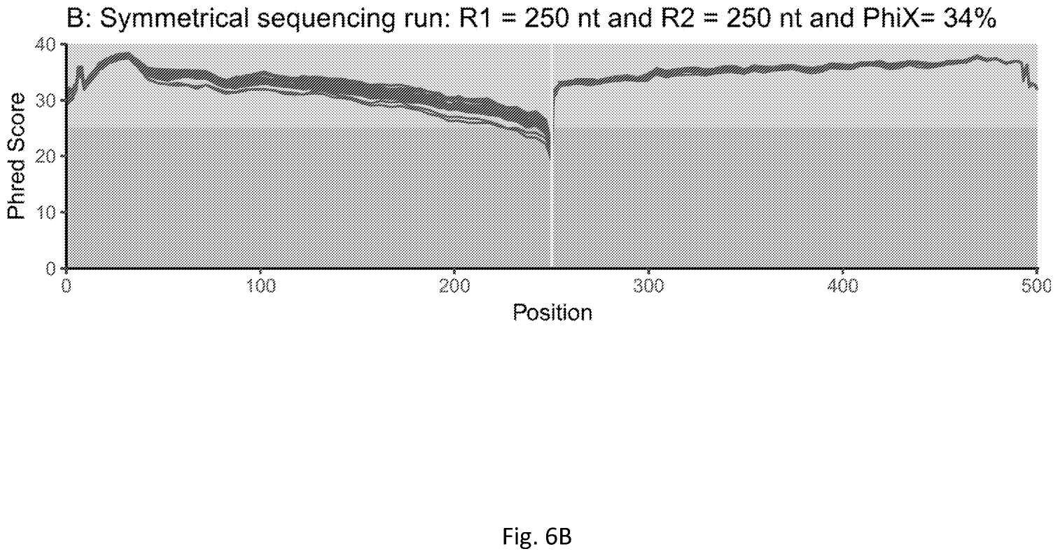

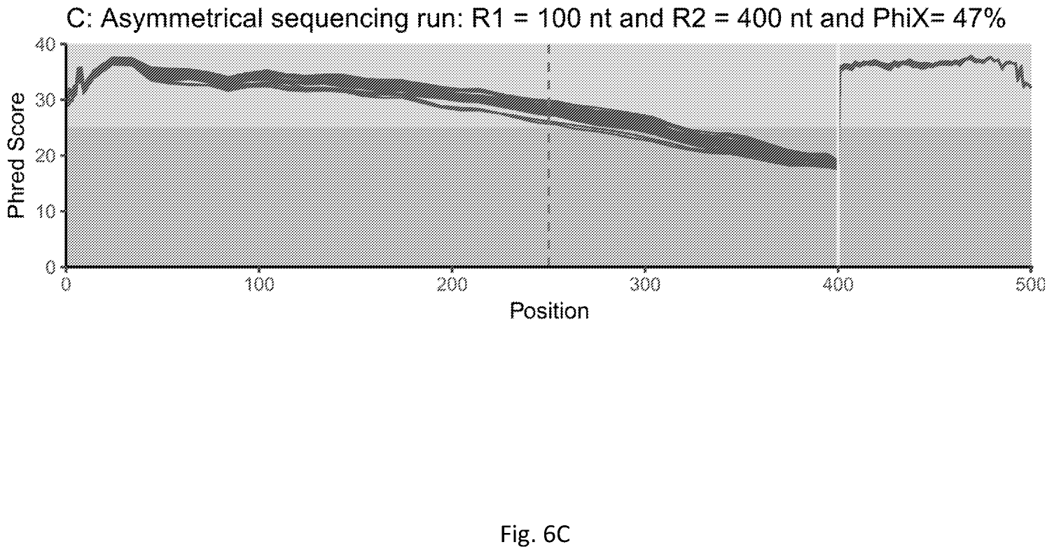

FIGS. 6A-6C illustrate exemplary sequencing reads according to embodiments of the present disclosure. FIG. 6A shows a symmetrical sequencing run where read 1 (R1) equals 250 nt and read 2 (R2) equals 250 nt and PhiX is equal to 20%. FIG. 6B shows a symmetrical sequencing run where read 1 (R1) equals 250 nt and read 2 (R2) equals 250 nt and PhiX is equal to 34%. FIG. 6C shows an asymmetrical sequencing run where read 1 (R1) equals 100 nt and read 2 (R2) equals 400 nt and PhiX is equal to 47%.

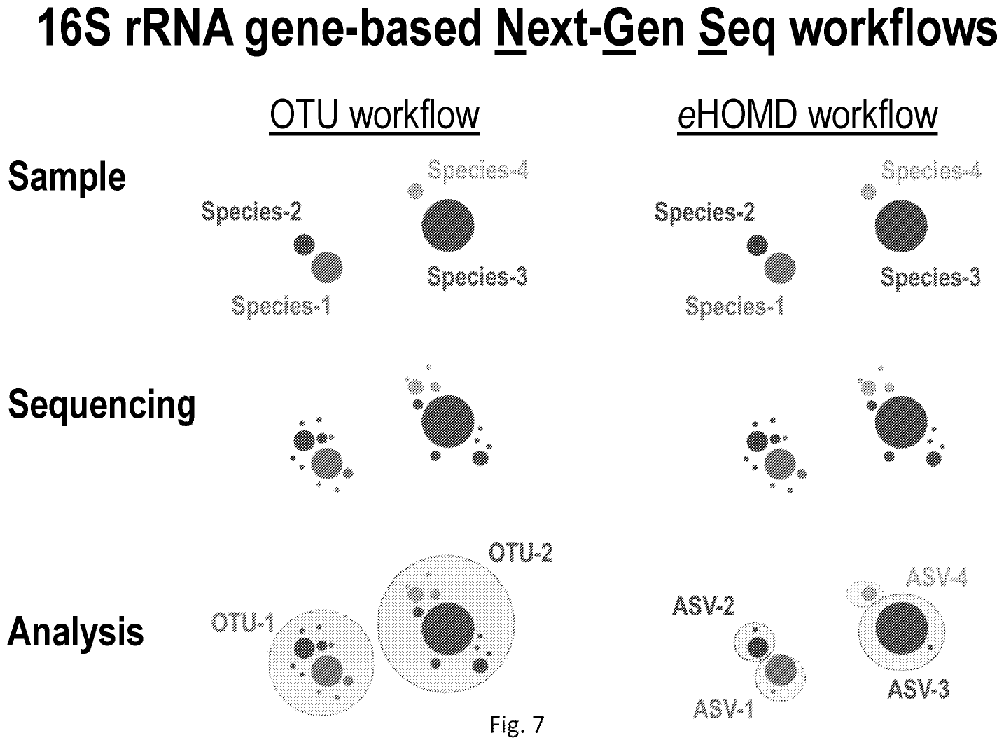

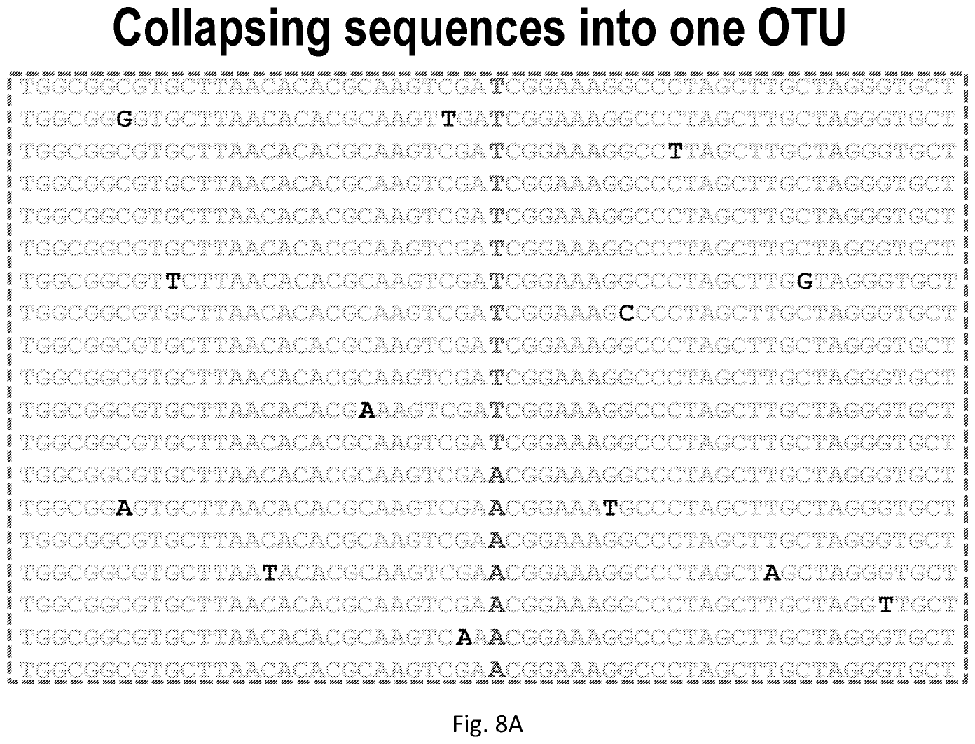

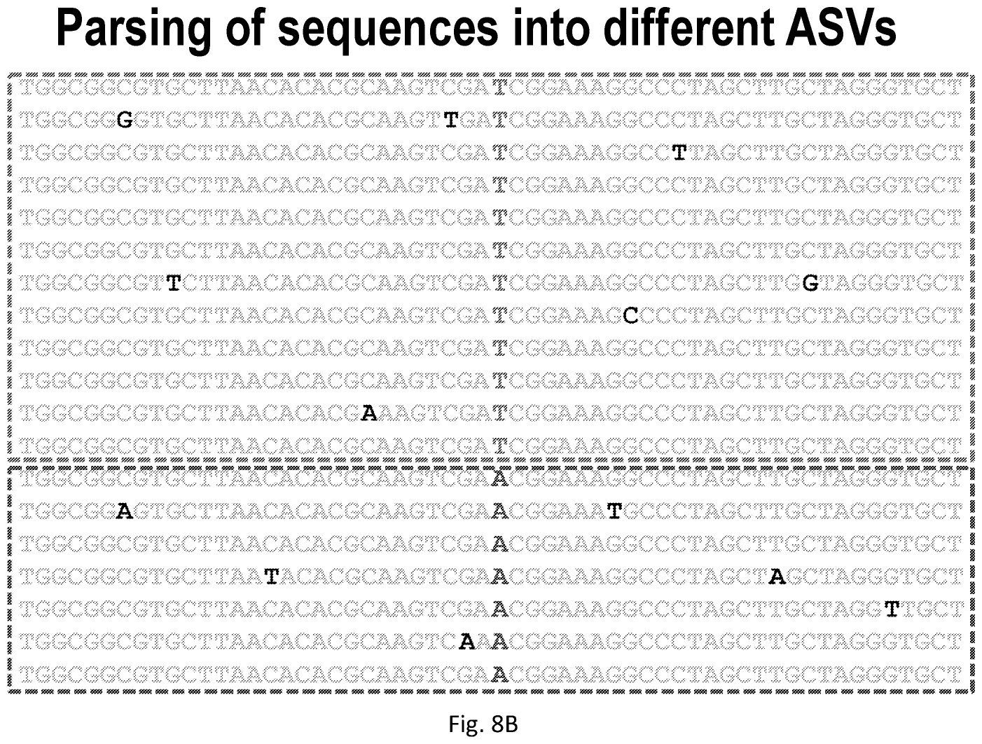

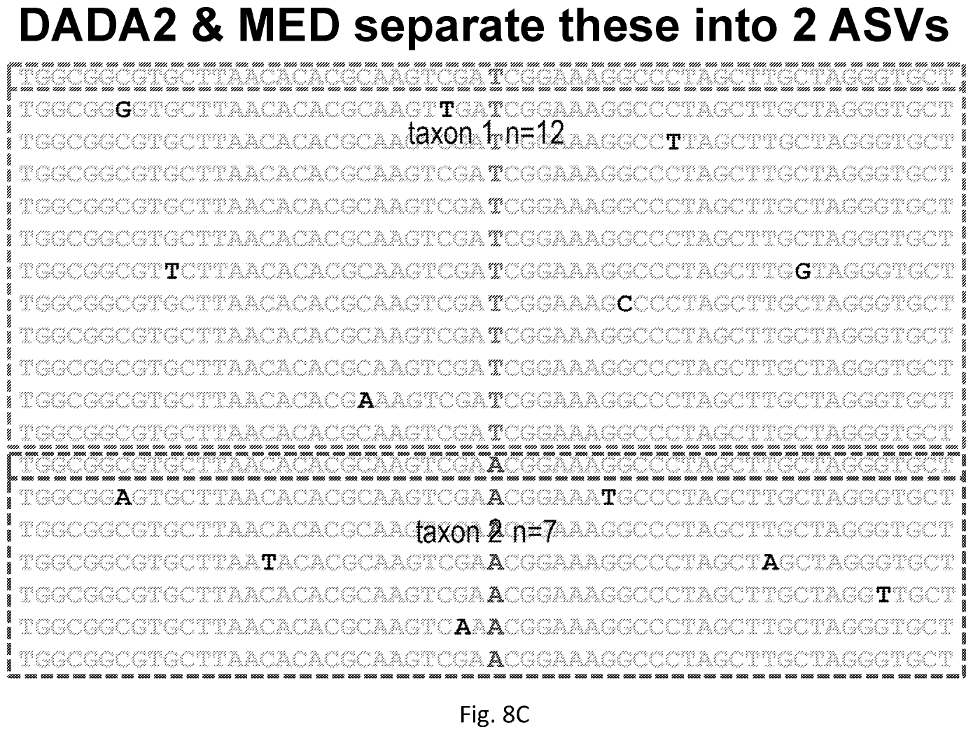

FIG. 7 illustrates a comparison of a OTU workflow and an eHOMD workflow according to embodiments of the present disclosure. In particular, FIG. 8 illustrates the differences between an OUT workflow and an eHOMD workflow. In both workflows, species 1-4 are sampled from a habitat, and genetic material (that is purified and amplified) is sequenced in a sequencer (e.g., next-gen sequencer). With an OTU workflow, two OTUs are generated where the OTU-1 encompasses species 1 and 2 while OTU-2 encompasses species 3 and 4. The OTU workflow thus fails to differentiate between species 1 and 2 and species 3 and 4. In the eHOMD workflow, however, taxons 1-4 are generated using an analysis of the 16S region with OUT binning at 97%. Taxon 1-4 accurately identify species 1-4 from the sampled habitat. In various embodiments, sequencing may be performed on the 16S region. In various embodiments, analysis may include analyzing the 16S region with OTU binning of sequencing reads having 97% similarity.

In various embodiments, the sample step represents the real microbial community composition. In various embodiments, the dots represent different species, the size of the dots represents their absolute abundance, and the separation between them represents the phylogenetic distance. In various embodiments, the sequencing step illustrates noise (e.g., errors) that may be generated during the library preparation and sequencing. In various embodiments, traditional OTU analysis pipelines collapse several species on the same OTU (for example, species 1 and 2 are collapsed into a single OTU including the small noise/error dots). In various embodiments, using high-resolution algorithms (e.g., MED or DADA2) instead of grouping reads into OTUs, species level information may be retained.

FIGS. 8A-8C illustrate various sequences according to embodiments of the present disclosure. In particular, FIG. 8A illustrates an example of how the sequences in the species 1 and 2 dots (including surrounding small dots due to noise/error) from FIG. 7 are collapsed into one OTU. FIG. 8B illustrates an example of how a high-resolution algorithm may separate the species 1 and 2 sequences into different ASVs based on highly informative regions of the sequence (e.g., the 16S region). As shown in FIGS. 8A-8C, most of the reads in the species 1 ASV are only one nucleotide apart from the ones in the species 2 ASV, but there is a highly informative position that makes evident that there are two types of reads here and, thus, the reads should not be collapsed as the same OTU. In various embodiments, algorithms such as, for example, MED or DADA2, may be able to differentiate reads into individual taxons (e.g., taxon 1 and 2) as shown in FIG. 8C.

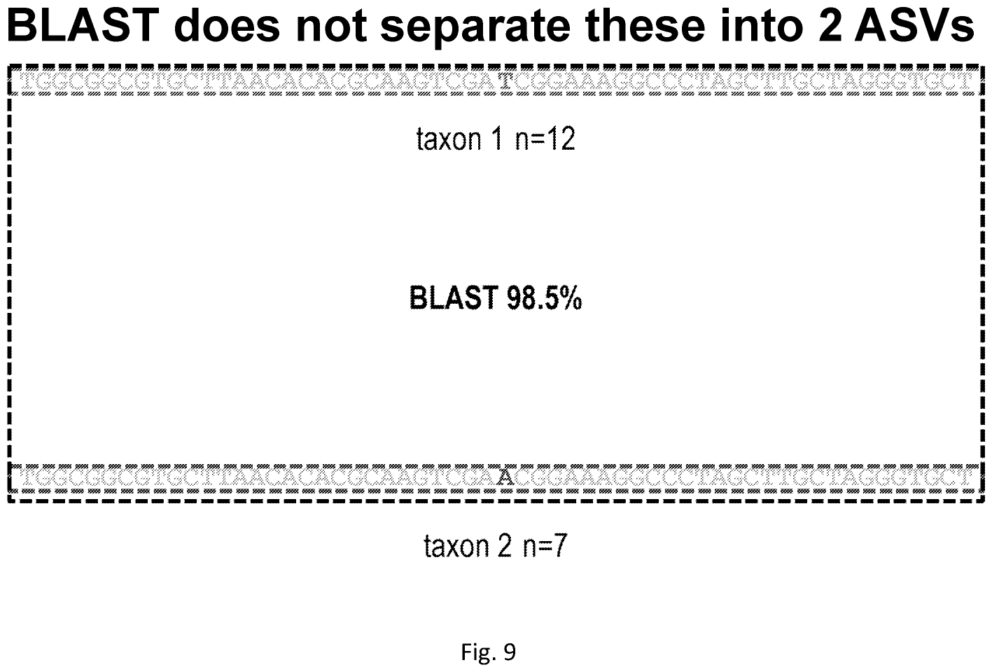

FIG. 9 illustrates a taxonomy assignment of various sequences according to embodiments of the present disclosure. In particular, FIG. 9 shows that when some algorithms are used to classify ASVs, e.g., a basic local alignment search tool (BLAST) for nucleotides, the algorithm may not be able to differentiate two ASVs (taxon 1 and taxon 2) from one another. In various embodiments, these algorithms may not be able to differentiate two different ASVs even at a similarity of 98.5%. In contrast, algorithms as described above (DADA2 and/or MED) may be capable of differentiating the two ASVs and, thus, may be preferable to algorithms such as BLAST for classification of ASVs.

In various embodiments, a taxonomy assignment algorithm may be applied to sequence data that uses positional information. For example, the naïve Bayesian classifier may be used to classify sequence data. In various embodiments, the Naïve Bayesian classifier may be from the ribosomal data project (RDP).

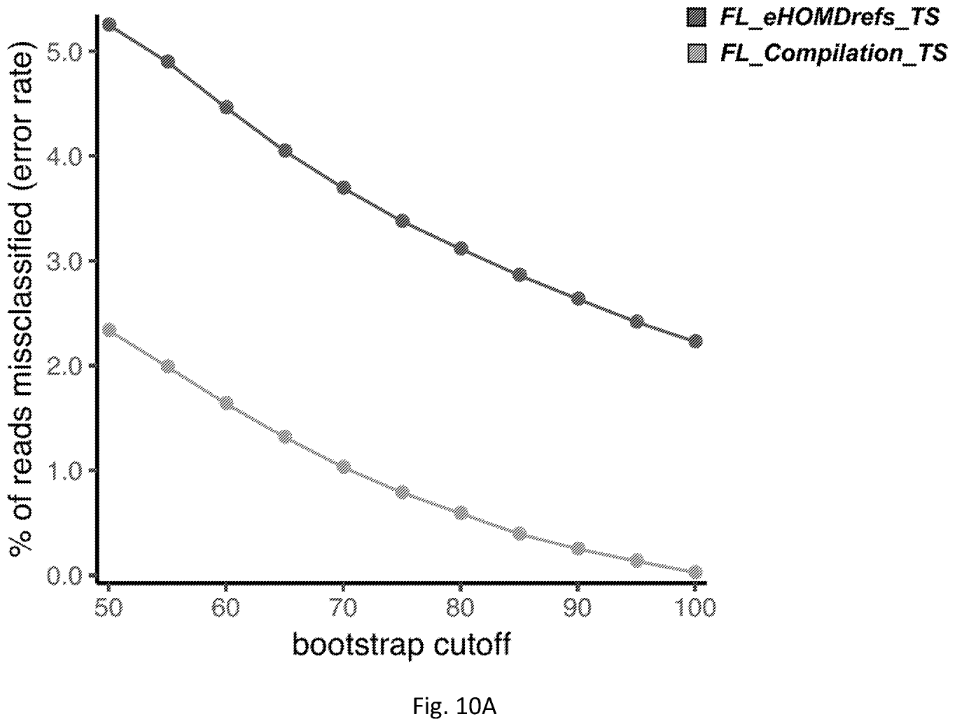

FIG. 10A illustrates a graph of reads misclassified according to embodiments of the present disclosure. FIG. 10B illustrates a graph of reads meeting a bootstrap threshold according to embodiments of the present disclosure. In the figures, FL_eHOMDref refers to full length reference sequences, while FL_Compilation_TS refers to the full length sequences related to each reference sequence by a similarity measure. In various embodiments, a reference sequence may be used. In various embodiments, a cluster of sequences that are approximately (e.g., 99%) identical to the reference sequence may be used. In various embodiments, a specific fragment of the gene (e.g., V1V3) may be sequenced instead of sequencing the full length.

In particular, FIGS. 10A-10B show the FL_Compilation_TS training set provides higher classification percentages with a lower error rate. The naïve Bayesian RDP Classifier was used with bootstrap values ranging from 50 to 100. FIG. 10A shows the percentage of eHOMD-derived simulated reads classified using the FL_eHOMDrefs_TS training set versus the FL_Compilation_TS training set. FIG. 10B shows the percentage of classified reads that were misclassified (i.e., reads for which the assigned taxonomic identity was different than the known identity of the original sequence from which the simulated read was derived).

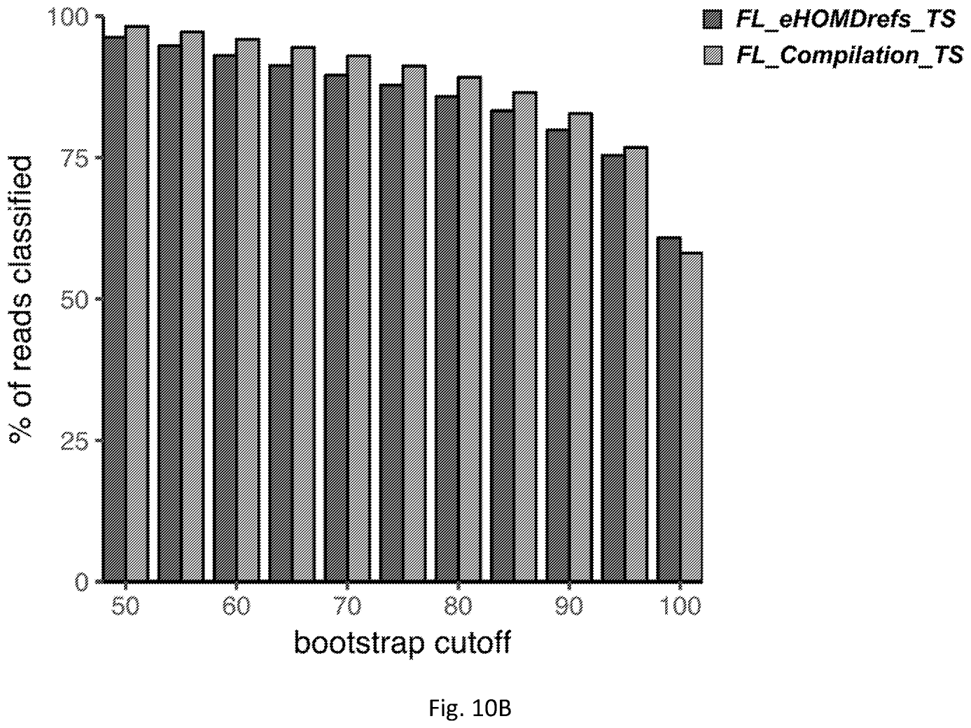

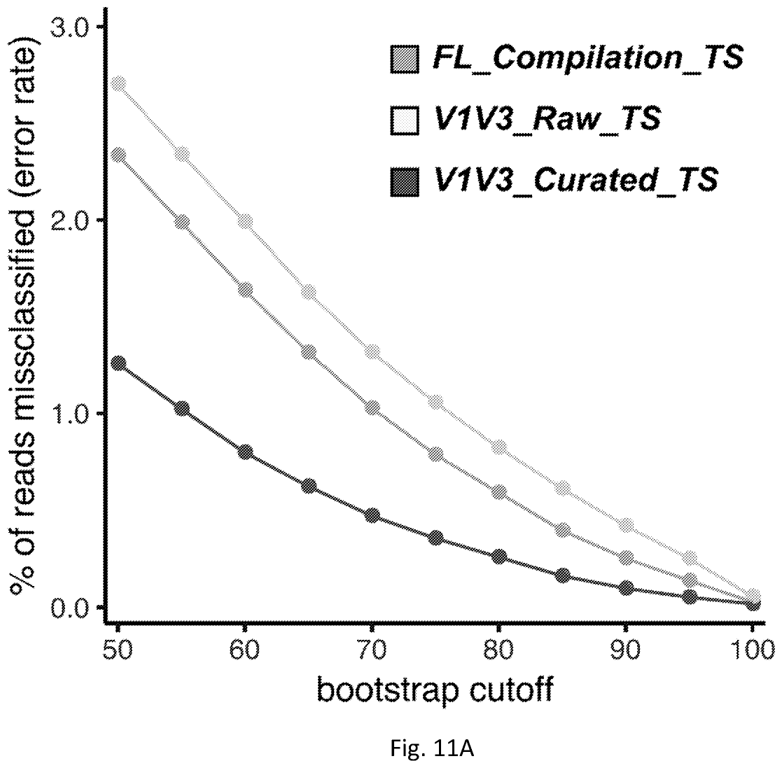

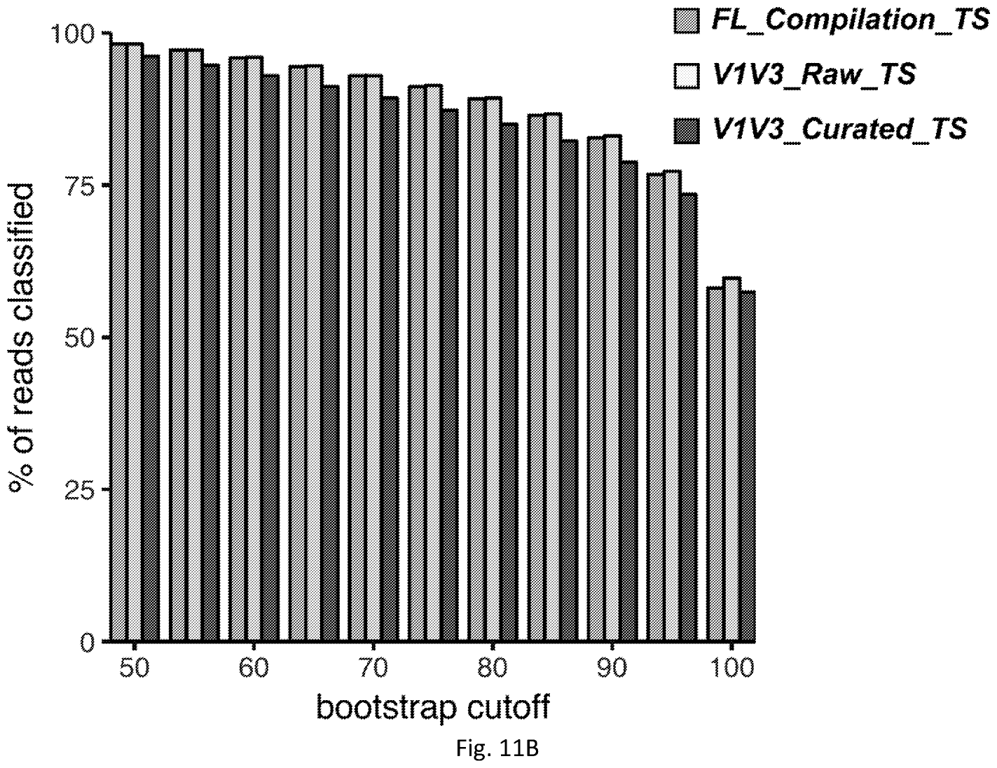

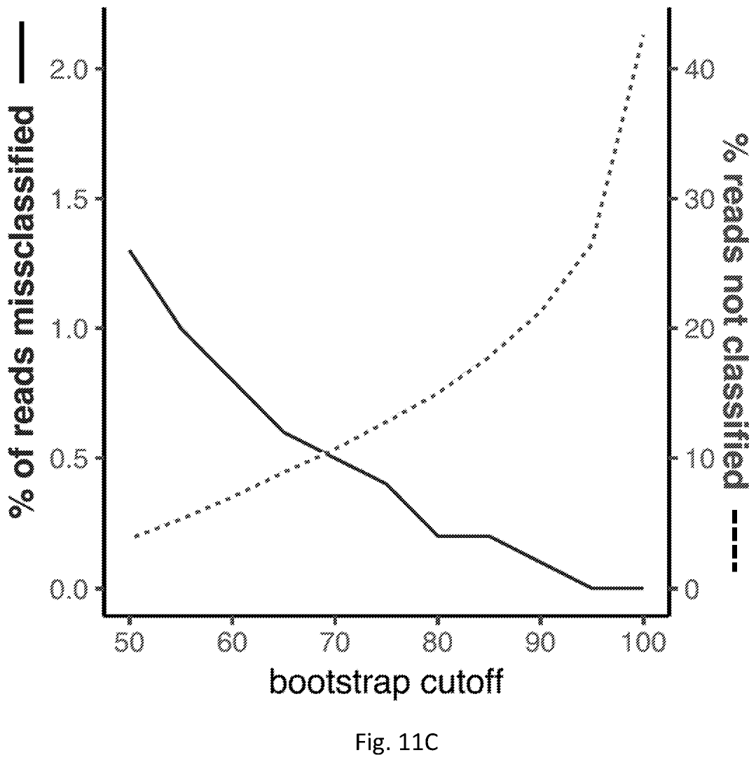

FIG. 11A illustrates a graph of reads misclassified when using V1V3 instead of full length sequences according to embodiments of the present disclosure. Comparison among truncated sequences is advantageous with respect to computation complexity. Moreover, as shown herein, truncation to V1V3 leads to fewer misreads due to do trivial variation outside of V1V3. FIG. 11B illustrates a graph of reads meeting a bootstrap threshold according to embodiments of the present disclosure. In various embodiments, the specific fragment of the gene that is analyzed may be cleaned. In various embodiments, cleaning may include, e.g., collapsing identical sequences, creating credible joint taxa, and discarding spurious data. FIG. 11C illustrates a graph of reads misclassified and reads not called according to embodiments of the present disclosure.

In particular, FIGS. 11A-11C show trimming the training set to the specific sequenced region further reduces the error rate. FIG. 11A shows the percentage of eHOMD-derived simulated reads classified at species level using the FL_Compilation_TS training set compared to subsequent trimmed versions V1V3_Raw_TS and V1V3_Curated_TS. FIG. 11B shows the percentage of classified reads that were misclassified with each of these three training sets. The naïve Bayesian RDP Classifier was used with bootstrap values ranging from 50 to 100. FIG. 11C shows a graph, which is specific to the eHOMD training set construction (V1V3_eHOMDSim_250N100 dataset), that indicates how researchers can determine the bootstrap value to use with the naïve Bayesian RDP Classifier by deciding an acceptable level of the % of reads misclassified (e.g., 0.5%) and/or of the % of reads that are not classified.

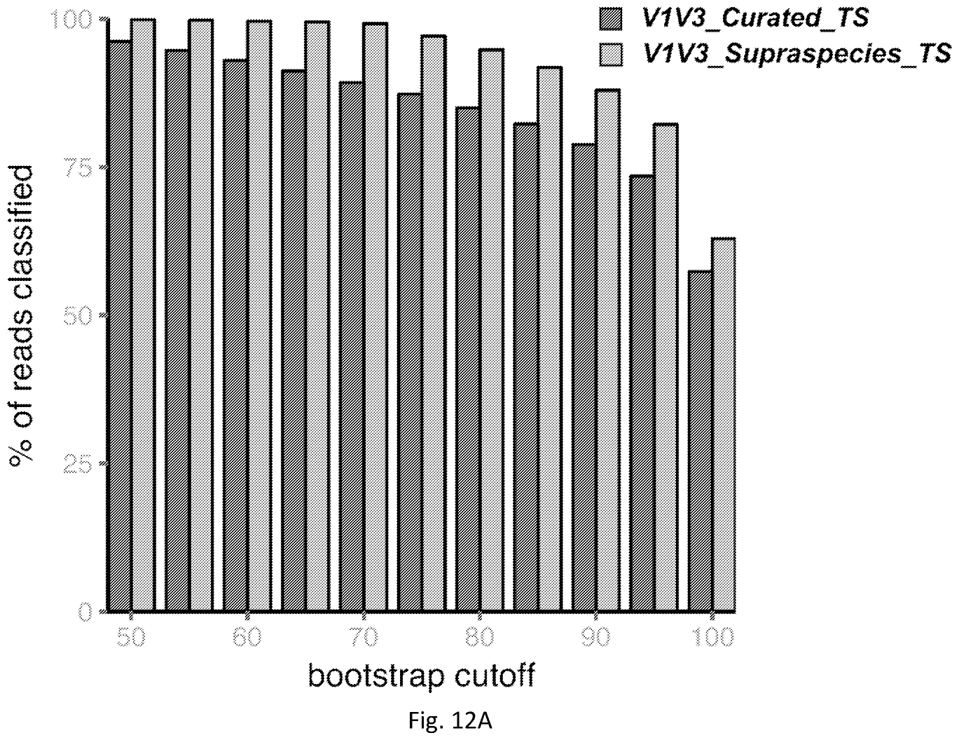

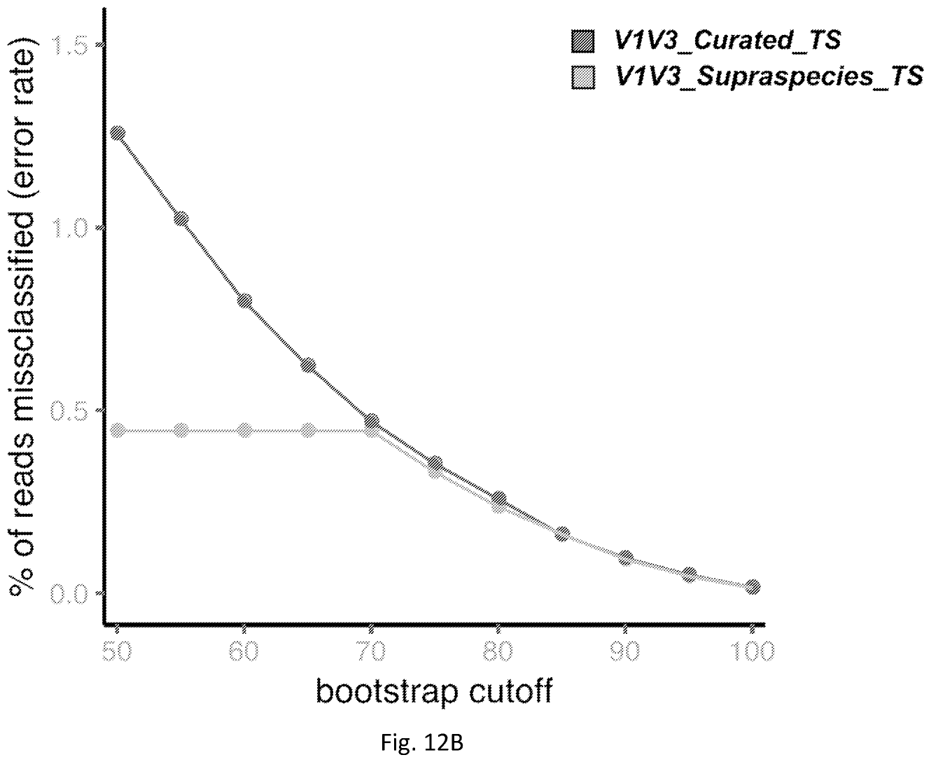

FIG. 12A illustrates a graph of reads meeting a bootstrap threshold according to embodiments of the present disclosure. FIG. 12B illustrates a graph of reads misclassified according to embodiments of the present disclosure. In particular, FIGS. 12A-12B show the addition of a supraspecies level to the training set increases the percentage of classified reads. (a) The percentage of eHOMD-derived simulated reads classified at species/supraspecies level using the V1V3_Curated_TS training set (red) versus the FL_Supraspecies_TS training set. (b) Percentage of classified reads that were misclassified with each of these TS. The naïve Bayesian RDP Classifier was used with bootstrap values ranging from 50 to 100.

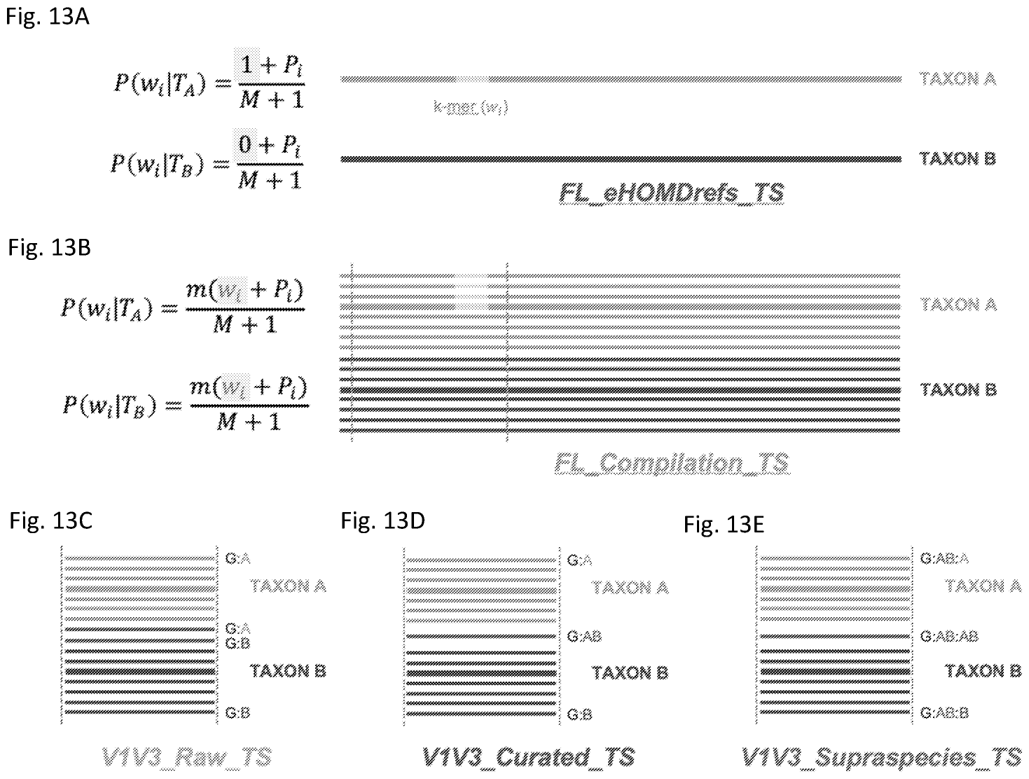

FIGS. 13A-13E illustrates various clusters of sequences according to embodiments of the present disclosure. The bold lines signify the reference sequences for exemplary Taxa A and B. FIG. 13A, gives the conditional probability of Taxa A and B, for the full length reference sequences, as described further above. In FIG. 13B, members of Taxa A and B are arranged around the reference sequences according to their similarity to those sequences. Referring to FIG. 13C, two exemplary sequences are close to the reference sequence for both Taxa A and B, after truncation to V1V3. Accordingly, as shown in FIG. 13D, this intermediate sequence is tagged with combined taxon AB. In some embodiments, taxon AB is considered to be hierarchically a super category of the taxa A and B. Accordingly, in FIG. 13D, members of Taxa A and B are also members of super-taxon AB. As noted herein, in some embodiments, the taxon may correspond to a species while the super taxon may correspond to a supraspecies.

In particular, FIGS. 13A-13E show a schematic representation of the steps to generate sequential habitat-specific training sets. FIG. 13A shows the FL_eHOMDrefs_TS training set contains all full-length eHOMDrefs (thick lines) from eHOMDv15.1 together with their respective taxonomic assignment. When only one read represents each taxon (M=1) a given k-mer can only be either present (1) or absent (0). FIG. 13B shows a higher number of sequences per taxon (M) allows for better resolution on the assignment, with the presence of a given k-mer across each cluster of reads (wi) being represented as a proportion out of the total number of reads in that taxon (M). Therefore, to better represent the known sequence diversity of the 16S rRNA gene(s) for each taxon, the training set FL_Compilation_TS includes clusters of sequences (thin lines) recovered from the NCBI nonredundant nucleotide (nr/nt) database that matched with 99% identity and ≥98% coverage (see methods) to each eHOMDref (thick line). FIG. 13C shows the training set V1V3_Raw_TS is a V1-V3 trimmed version of the FL_Compilation_TS training set. The schematic illustrates how trimming to this region leads to identical reads having two different taxonomic designations. Here, G is genus and species are labeled as A or B. FIG. 13D shows to construct the V1V3_Curated_TS training set, identical V1-V3 sequences in the V1V3_Raw_TS training set were collapsed into one. If identical sequences came from more than one taxon, species-level names of all taxa involved were concatenated (AB). FIG. 13E shows the V1V3_Supraspecies_TS training set includes the same sequences that the V1V3_Raw_TS training set; however, the headers in the fasta file include the supraspecies taxon (AB) as an extra level between the genus (G) and species taxonomic levels (A, B or AB), as illustrated here.

In various embodiment, this process may use a cluster of sequences that are 99% identical to a full length reference sequence. In various embodiments, a reference sequence has an associated label. For example, the label may identify a given taxon, such as a genus or species, to which the reference sequence belongs. Additional sequences may be compared to the reference sequences, for example on a pairwise basis, in order to determine clusters of similar sequences. In some embodiments, a 99% similarity threshold is imposed to define a cluster around a reference sample. However, it will be appreciated that a variety of alternative thresholds may be imposed. For example, a 98% or 98.5% threshold may be imposed.

In various embodiment, a supraspecies level may be introduced in classification of clusters of sequences according to embodiments of the present disclosure. In various embodiments, for those sequences that are highly similar, a combined taxon may be formed. For example, where two sequences have been assigned difference labels, but have greater than a predetermined similarity, they may be assigned to a combined taxon. In this example, those sequences which would have been assigned to species A or B, but which are highly similar to each other, are instead assigned to a combined species AB. This combines species is referred to herein as a supraspecies or supraspecies, as it spans more than one species, and thus lies between genus and species in terms of breadth.

FIG. 13A illustrates various clusters of sequences where the bolded (i.e., thicker) lines signify the reference sequences for exemplary Taxa A and B. In FIG. 13B, members of Taxa A and B are arranged around the reference sequences according to their similarity to those sequences. Referring to FIG. 13C, two exemplary sequences are close to the reference sequence for both Taxa A and B. Accordingly, as shown in FIG. 13D, this intermediate sequence is tagged with combined taxon AB. In some embodiments, taxon AB is considered to be hierarchically a supercategory of the taxa A and B. Accordingly, in FIG. 13D, members of Taxa A and B are also members of super-taxon AB. As noted herein, in some embodiments, the taxon may correspond to a species while the super taxon may correspond to a supraspecies.

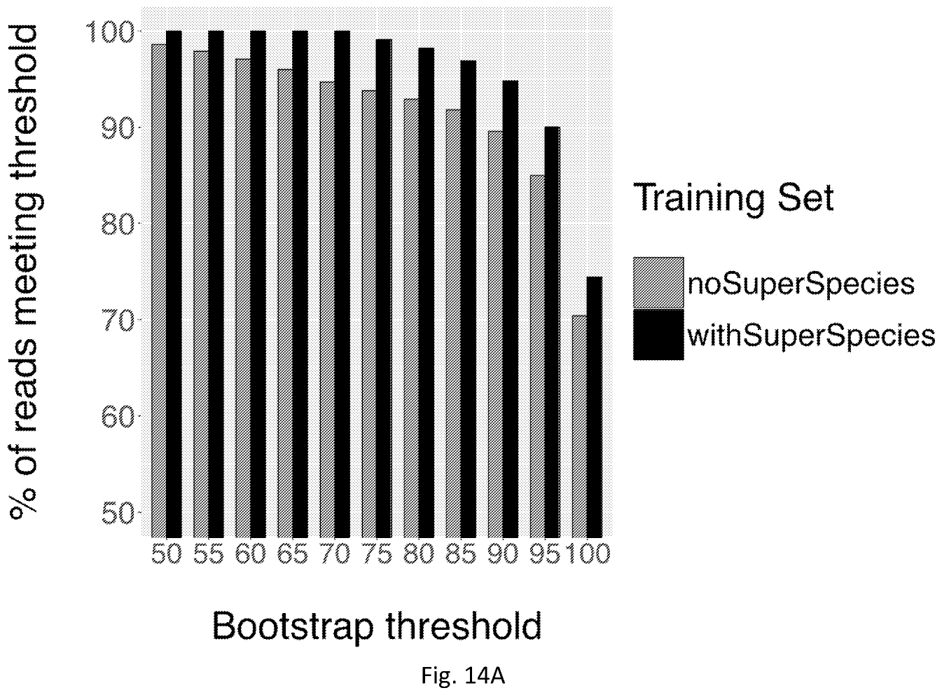

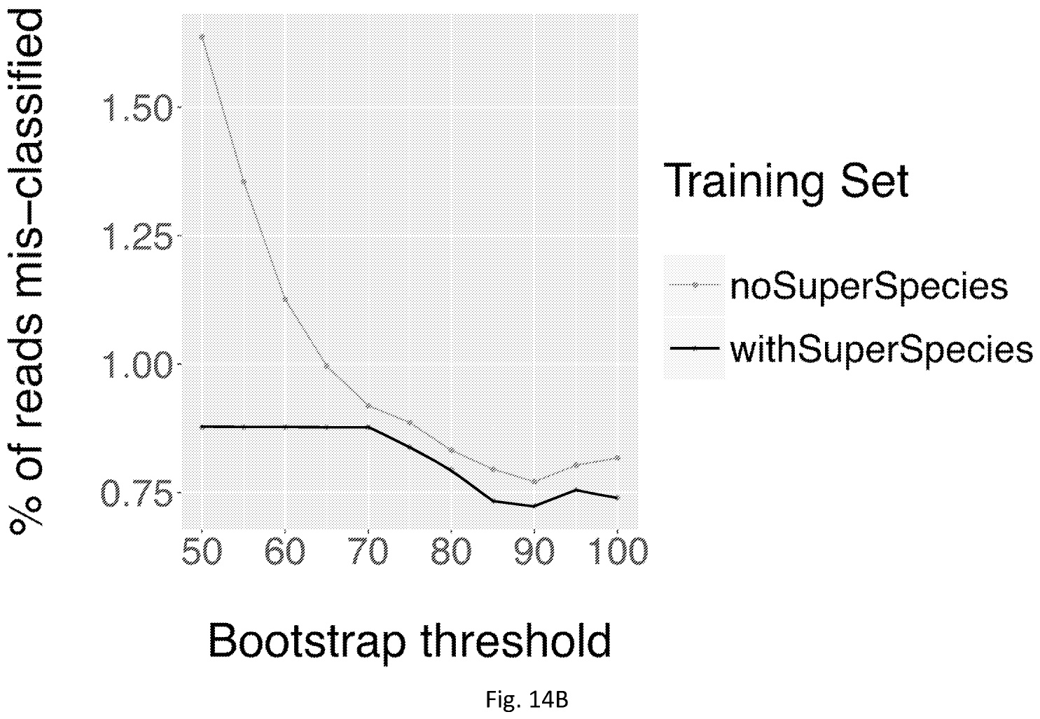

FIG. 14A illustrates a graph of reads meeting a bootstrap threshold according to embodiments of the present disclosure. FIG. 14B illustrates a graph of reads misclassified according to embodiments of the present disclosure. As shown, the addition of supraspecies leads to higher accuracy. Significant gains in accuracy (i.e., lower % of reads misclassified) are observed for bootstrap thresholds of 50 to 70 and an approximately constant gain in accuracy is observed at a bootstrap level of 70 and 100.

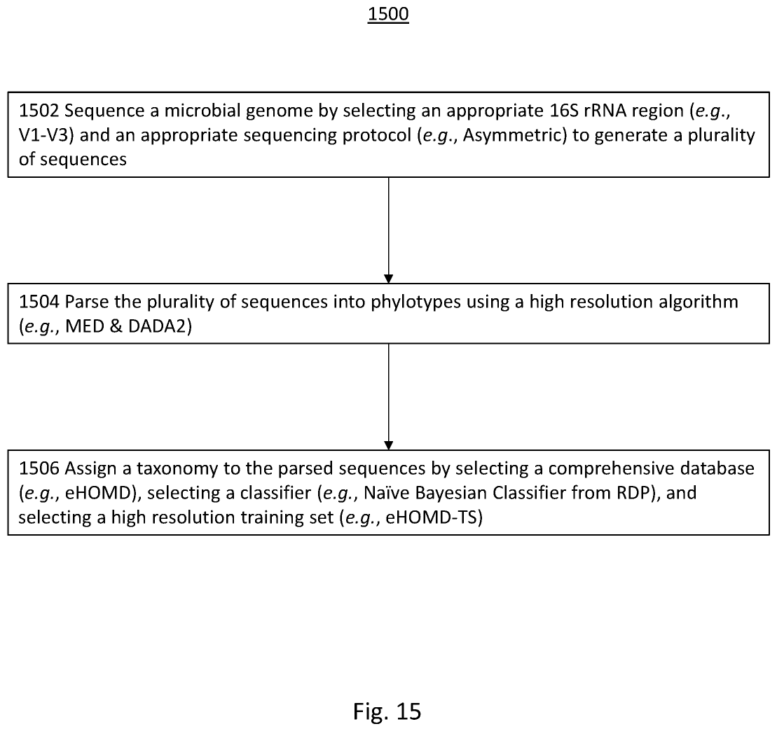

FIG. 15 illustrates a method 1500 for species-level rRNA analysis according to embodiments of the present disclosure. At 1502, a microbial genome is sequenced by selecting an appropriate 16S rRNA region (e.g., V1-V3) and an appropriate sequencing protocol (e.g., Asymmetric) to generate a plurality of sequences. At 1504, the plurality of sequences are parsed into phylotypes using a high resolution algorithm (e.g., MED & DADA2). At 1506, a taxonomy is assigned to the parsed sequences by selecting a comprehensive database (e.g., eHOMD), selecting a classifier (e.g., Naïve Bayesian Classifier from RDP), and selecting a high resolution training set (e.g., eHOMD-TS).

FIG. 16 illustrates a method 1600 for species-level rRNA analysis according to embodiments of the present disclosure. At 1602, a plurality of reference sequences are received. Each of the plurality of reference sequences corresponds to a taxonomical classification. At 1604, a label corresponding to at least one of the reference sequences is assigned to each of a plurality of supplemental sequences. At 1606, each of the plurality of supplemental sequences and each of the plurality of reference sequences are truncated to a region of interest to thereby generate a truncated set of sequences. At 1608, similarity is measured between pairs of truncated sequences in the truncated set of sequences to determine whether the similarity is above a predetermined threshold. At 1610, an intermediate taxonomical label is assigned to the pair of truncated sequences in the truncated set of sequences when the similarity is above the predetermined threshold to thereby generate an enhanced set of sequences.

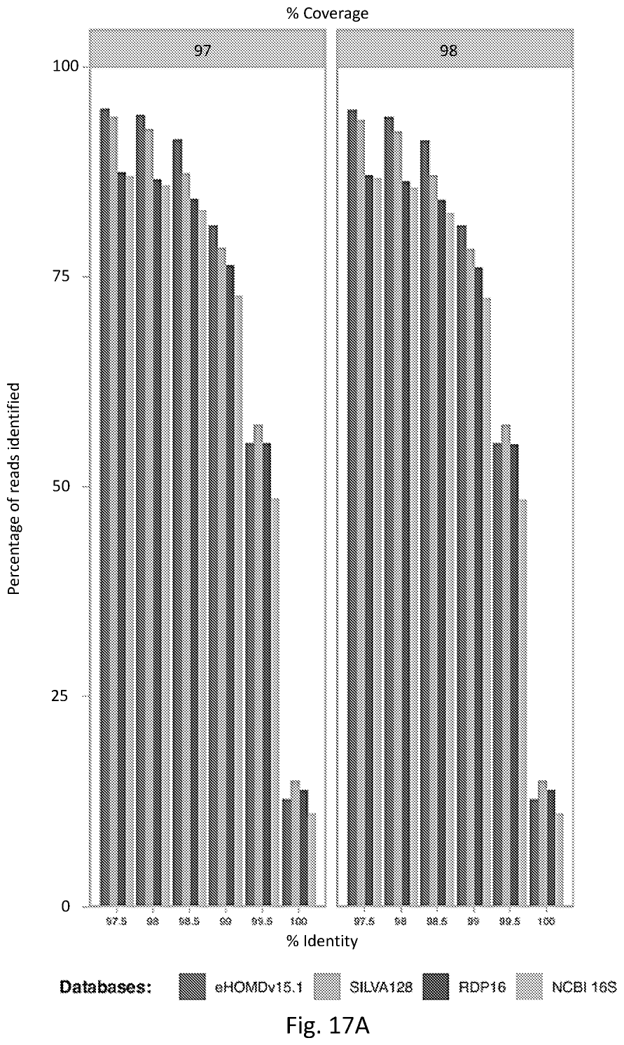

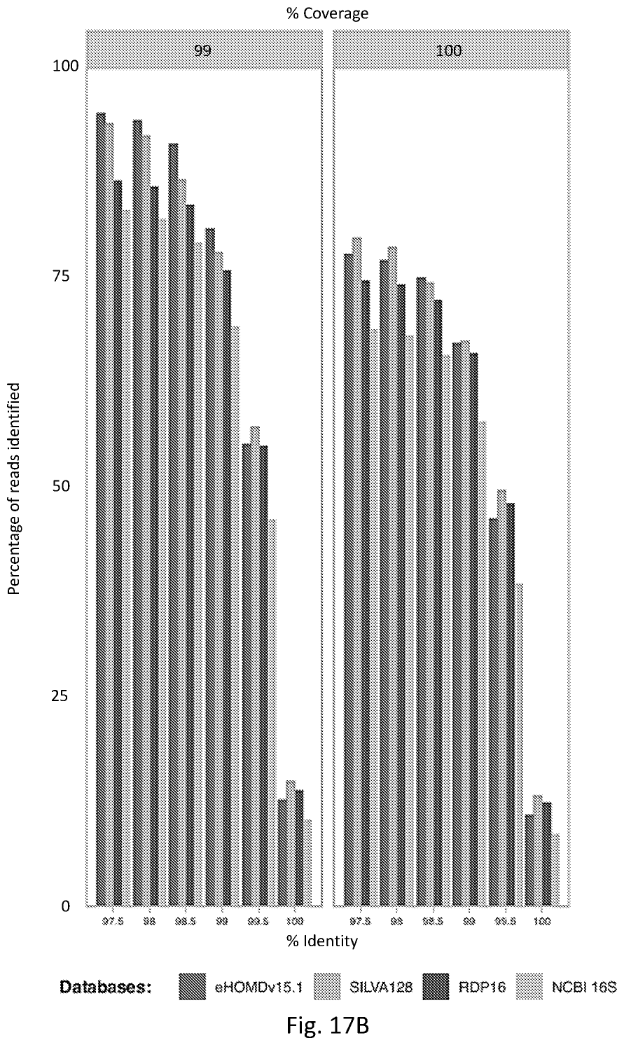

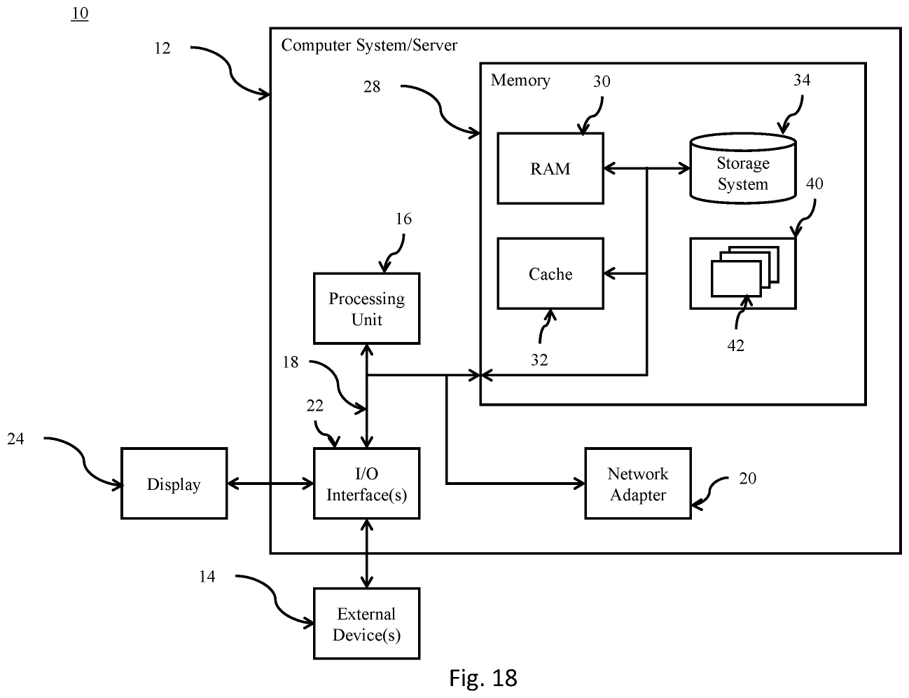

FIGS. 17A-17B illustrate exemplary graphs of the percentage of 16S rRNA gene sequences identified via blastn for the HMP nares V1-V3 rRNA dataset according to embodiments of the present disclosure. In particular, the percentage of 16S rRNA gene sequences identified via blastn declines sharply at identity thresholds above 98.5% across the range of coverage tested. Blastn results of the HMP nares V1-V3 16S rRNA dataset, as an example of a short NGS-generated dataset, are compared against four different databases. The grey panels on top show the range of % coverage used. The x-axis represents the range of % identity thresholds used. Each database is represented in a different color (see key). In various embodiments, a threshold of 98.5% identity and 98% coverage for blastn analysis may be selected. Data used to generate FIGS. 17A and 17B may be found in Tables 1 and 2 below.

The expanded Human Oral Microbiome Database (eHOMD) is a comprehensive microbiome database for sites along the human aerodigestive tract that revealed new insights into the nostril microbiome. The eHOMD provides well-curated 16S rRNA gene reference sequences linked to available genomes and enables assignment of species-level taxonomy to most NextGeneration sequences derived from diverse aerodigestive tract sites, including the nasal passages, sinuses, throat, esophagus and mouth. Using Minimum Entropy Decomposition coupled with the RDP Classifier and our eHOMD V1-V3 training set, we reanalyzed 16S rRNA V1-V3 sequences from the nostrils of 210 Human Microbiome Project participants at the species level revealing four key insights. First, we discovered that Lawsonella clevelandensis, a recently named bacterium, and Neisseriaceae [G-1] HMT-174, a previously unrecognized bacterium, are common in adult nostrils. Second, just 19 species accounted for 90% of the total sequences from all participants. Third, one of these 19 belonged to a currently uncultivated genus. Fourth, for 94% of the participants, two to ten species constituted 90% of their sequences, indicating nostril microbiome may be represented by limited consortia. These insights highlight the strengths of the nostril microbiome as a model system for studying interspecies interactions and microbiome function. Also, in this cohort, three common nasal species (Dolosigranulum pigrum and two Corynebacterium species) showed positive differential abundance when the pathobiont Staphylococcus aureus was absent, generating hypotheses regarding colonization resistance. By facilitating species-level taxonomic assignment to microbes from the human aerodigestive tract, the eHOMD is a vital resource enhancing clinical relevance of microbiome studies.

The eHOMD is a valuable resource for researchers, from basic to clinical, who study the microbiomes, and the individual microbes, in health and disease of body sites in the human aerodigestive tract, which includes the nasal passages, sinuses, throat, esophagus and mouth, and also provides coverage of the lower respiratory tract. The eHOMD is an actively curated, web-based, open-access resource. The eHOMD provides the following: (1) species-level taxonomy based on grouping 16S rRNA gene sequences at 98.5% identity, (2) a systematic naming scheme for unnamed and/or uncultivated microbial taxa, (3) reference genomes to facilitate metagenomic, metatranscriptomic and proteomic studies and (4) convenient cross-links to other databases (e.g., PubMed and Entrez). By facilitating the assignment of species names to sequences, the eHOMD is a vital resource for enhancing the clinical relevance of 16S rRNA gene-based microbiome studies, as well as metagenomic studies. The eHOMD provisional naming scheme may permit cross-study comparison of 16S rRNA gene sequences from both formally named species and as-yet-unnamed or uncultivated species.

### Results and Discussion

I. The eHOMD is a Resource for Microbiome Research on the Human Upper Digestive and Respiratory Tracts.

As described below, the eHOMD is a comprehensive, actively curated, web-based resource open to the entire scientific community that classifies 16S rRNA gene sequences at a high resolution (98.5% sequence identity). Further, the eHOMD provides a systematic provisional naming scheme for as-yet unnamed/uncultivated taxa and a resource for easily searching available genomes for included taxa, thereby, facilitating the identification of aerodigestive and lower respiratory tract bacteria and providing phylogenetic, genomic, phenotypic, clinical and bibliographic information for these microbes.

The eHOMD captures the breadth of diversity of the human nostril microbiome. Here we describe the generation of eHOMDv15.1, which performed as well or better than four other commonly used 16S rRNA gene databases (SILVA128, RDP16, NCBI 16S and Greengenes GOLD) in assigning species-level taxonomy via blastn to sequences in a dataset of nostril-derived 16S rRNA gene clones (Table 1) and short-read fragments (Table 2). Species-level taxonomy assignment was defined as 98.5% identity with 98% coverage via blastn. An initial analysis showed that the oral-focused HOMDv14.5 enabled species-level taxonomic assignment of only 50.2% of the 44,374 16S rRNA gene clones from nostril (anterior nares) samples generated by Julie Segre, Heidi Kong and colleagues, henceforth the SKn dataset (Table 1).

TABLE 1
The eHOMD outperforms comparable databases for
species-level taxonomic assignment to 16S rRNA
reads from nostril samples (SKn dataset).

| | # Reads | % Reads |
|---|---|---|
| Database | Identified^{a} | Identified^{a} |
| HOMDv14.5 | 22,274 | 50.2 |
| eHOMDv15.1 | 42,197 | 95.1 |
| SILVA128 | 40,597 | 91.5 |
| RDP16 | 38,815 | 87.5 |
| NCBI 16S | 38,337 | 86.4 |
| Greengenes GOLD | 31,195 | 70.3 |

^{a}Reads identified via blastn at 98.5% identity and 98% coverage

TABLE 2
Performance of eHOMD and comparable databases for species-level taxonomic assignment
to 16S rRNA gene datasets from sites throughout the human aerodigestive tract.

| | 16S | 16S | Sequecing | | | # Reads | | # Reads | % Reads |
|---|---|---|---|---|---|---|---|---|---|
| Dataset | Region | Primers | Technique | Sample Type | # Samples | analyzed | Database | Identified^{a} | Identified^{a} |
| Laufer- | V1-V2 | 27F 338R | Roche/454 | Nostril | 108 children | 120274 | eHOMDv15.1 | 96233 | 80.0 |
| Pettigrew | | | | swab | (108 samples) | | SILVA128 | 97233 | 80.8 |
| (2011) | | | | | | | RDP16 | 97464 | 81.0 |
| | | | | | | | NCBI 16S | 87082 | 72.4 |
| Allen-Sale | V1-V3 | 27F 534R | 454-FLX | Nasal lavage | 10 adults | 75310 | eHOMDv15.1 | 68594 | 91.1 |
| (2014) | | | | fluid | (97 samples) | | SILVA128 | 69082 | 91.7 |
| | | | | | | | RDP16 | 65028 | 86.4 |
| | | | | | | | NCBI 16S | 63892 | 84.8 |
| Pei-Blaser | CL | 318F 1519R | CL | Esophageal | 4 (10 | 7414 | eHOMDv15.1 | 7276 | 98.1 |
| (2004; 2005) | | 8F 1513R | | biopsies | libraries | | SILVA128 | 7019 | 94.7 |
| | | | | | each) | | RDP16 | 6847 | 92.4 |
| | | | | | | | NCBI 16S | 6686 | 90.2 |
| Harris-Pace | CL | 27F 907R | CL | Brochial | 57 children | 3203 | eHOMDv15.1 | 2684 | 83.8 |
| (2007) | | | | alveolar | (50 libraries | | SILVA128 | 2633 | 82.2 |
| | | | | lavage | CF and 19 | | RDP16 | 2500 | 78.1 |
| | | | | fluid | control) | | NCBI 16S | 2427 | 75.8 |
| HMPnV1-V3 | V1-V3 | 27F 534R | Roche/454 | Nostril | 227 adults | 2338563 | eHOMDv15.1 | 2133083 | 91.2 |
| | | | | swab | (363 | | SILVA128 | 2035882 | 87.1 |
| | | | | | samples)^{b} | | RDP16 | 1965611 | 84.1 |
| | | | | | | | NCBI 16S | 1932732 | 82.6 |
| vanderGast- | CL | 7F 1510R | CL | Expectorated | 14 adults | 2137 | eHOMDv15.1 | 2123 | 99.3 |
| Bruce (2011) | | | | Sputa | (CF) | | SILVA128 | 2084 | 97.5 |
| | | | | | | | RDP16 | 2057 | 96.3 |
| | | | | | | | NCBI 16S | 2045 | 95.7 |
| Flanagan- | CL | 27F 1492R | CL | Endotracheal | 6 adults, 1 | 3278 | eHOMDv15.1 | 3193 | 97.4 |
| Bristow | | | | tube | children (2-5 | | SILVA128 | 3199 | 97.6 |
| (2007) | | | | aspirate | samples each) | | RDP16 | 3193 | 97.4 |
| | | | | | | | NCBI 16S | 3186 | 97.2 |
| Perkins- | CL | 8F 1391R | CL | Extubated | 8 adults | 1263 | eHOMDv15.1 | 1008 | 79.8 |
| Angenent | | | | endotracheal | | | SILVA128 | 1000 | 79.2 |
| (2010) | | | | tube | | | RDP16 | 916 | 72.5 |
| | | | | | | | NCBI 16S | 832 | 65.9 |

^{a}Reads identified via blastn at 98.5% identity and 98% coverage
^{b}See Supplemental Methods
CL = Clone library;
CF = Cystic Fibrosis

To expand HOMD to be a resource for the microbiomes of the entire human aerodigestive tract, we started with the addition of nasal- and sinus-associated bacterial species. As illustrated in FIGS. 1A-1D, and described in detail in the methods, a list of candidate nasal and sinus species gleaned from culture-dependent studies plus anaerobes cultivated from cystic fibrosis sputa were compiled (Table S1A). To assess which of these candidate species are most likely to be common members of the nasal microbiome, we used blastn to identify those taxa present in the SKn dataset. We then added one or two representative close-to-full-length 16S rRNA gene sequences (eHOMDrefs) for each of these taxa to a provisional expanded database (FIGS. 1A-1D). Using blastn, we assayed how well this provisional eHOMDv15.01 captured clones in the SKn dataset (Table SIB). Examination of sequences in the SKn dataset that were not identified resulted in further addition of new HMTs generating the provisional eHOMDv15.02 (FIGS. 1A-1D). Next, we evaluated how well eHOMDv15.02 served to identify sequences in the SKn clone dataset using blastn (FIGS. 1A-1D). To evaluate its performance for other datasets as compared to other databases, we took an iterative approach using blastn to evaluate the performance of eHOMDv15.02 against a set of three V1-V2 or V1-V3 16S rRNA gene short-read datasets and two close-to-full-length 16S rRNA gene clone datasets from the aerodigestive tract in children and adults in health and disease in comparison to three commonly used 16S rRNA gene databases: NCBI 16S Microbial (NCBI 16S), RDP16 and SILVA128 (FIGS. 1A-1D and Table SIC). These steps resulted in the generation of the provisional eHOMDv15.03. Further additions to include taxa that can be present on the skin of the nasal vestibule (nostril or nares samples) but which are more common at other skin sites resulted from using blastn to analyze the full Segre-Kong skin 16S rRNA gene clone dataset, excluding nostrils, (the SKs dataset) against both eHOMDv15.03 and SILVA128 (FIGS. 1C and 1D). Based on these results, we generated the eHOMDv15.1, which identified 95.1% of the 16S rRNA gene reads in the SKn dataset outperforming the three other commonly used 16S rRNA gene databases (Table 1). Importantly, examination of the 16S rRNA gene phylogenetic tree of all eHOMDrefs in eHOMDv15.1 demonstrated that this expansion maintained the previous distinctions among oral taxa with the exception of Streptococcus thermophiles, which is >99.6% similar to S. salivarius and S. vestibularis (Supplemental Data S1A and link to current version http://www.ehomd.org/ftp/HOMD_phylogeny/current). Each step in this process improved eHOMD with respect to identification of clones from the SKn dataset, establishing eHOMD as a resource for the human nasal microbiome (FIGS. 1A-1D and Table SIB).

SILVA128 identified the next largest percentage of the SKn clones (91.5%) at species-level by blastn with our criteria (Table 1). Of the 44,373 clones in the SKn dataset, a common set of 90.2% were captured at 98.5% identity and 98% coverage by both databases but with differential species-level assignment for 15.6% (6,237) (Table S2A). Another 1.3% were identified only with SILVA (Table S2B) and 4.9% were identified only with eHOMDv15.1 (Table S2C). Of the differentially named SKn clones, 45% belong to the genus Corynebacterium. Therefore, we generated a tree of all of the references sequences for Corynebacterium species from both databases (Supplemental Data SIB). This revealed that the C. jeikeium SILVA-JVVY01000068.479.1974 reference sequence clades with C. propinquum references from both databases, indicating a misannotation in SILVA128. This accounted for 34.4% (2,147) of the differentially named clones, which eHOMD correctly attributed to C. propinquum (Table S2A). Another 207 SKn clones were attracted to C. fastidiosum SILVA-AJ439347.1.1513. eHOMDv15.1 lacks this species, so incorrectly attributed 3.3% (207) to C. accolens. The bulk of the remaining differentially named Corynebacterium also resulted from misannotation of reference sequences in SILVA128, e.g., SILVA-JWEP01000081.32.1536 as C. urealyticum, JVX001000036.12.1509 as C. aurimucosum and SILVA-HZ485462.10.1507 as C. pseudogenitalium, which is not a validly recognized species name (Supplemental Data SIB). As described above, Edgar estimated an annotation error as high as ˜17% in comprehensive databases, e.g., SILVA128. Since eHOMD taxa are represented by just one to six highly curated eHOMDrefs, we minimize the misannotation issues observed in larger databases. At the same time, our deep analysis of the phylogenetic space of each taxon allows eHOMD to identify a high percentage of reads in aerodigestive tract datasets. Having compared eHOMDv15.1 and SILVA128, we next benchmarked the performance of eHOMDv15.1 for assigning taxonomy to both other 16S rRNA gene clone libraries and against short-read 16S rRNA fragment datasets from the human aerodigestive tract (Table 2).

The 16S rRNA gene V1-V3 region provides superior taxonomic resolution for bacteria from the human aerodigestive tract compared to the V3-V4 region that is commonly used in microbiome studies. The choice of variable region for NGS-based short-read 16S rRNA gene microbiome studies impacts what level of phylogenetic resolution is attainable. For example, for skin, V1-V3 sequencing results show high concordance with those from metagenomic sequencing. Similarly, to enable species-level distinctions within respiratory tract genera that include both common commensals and pathogens, V1-V3 is preferable for the nasal passages, sinuses and nasopharynx. In eHOMDv15.1, we observed that only 14 taxa have 100% identity across the V1-V3 region, whereas 63 have 100% identity across the V3-V4 region (Table 3). The improved resolution with V1-V3 was even more striking at 99% identity, with 37 taxa indistinguishable using V1-V3 compared to 269 indistinguishable using V3-V4. Table S3A-F shows the subsets of taxa collapsing into undifferentiated groups at each percent identity threshold for the V1-V3 and V3-V4 regions respectively. This analysis provides clear evidence that V1-V3 sequencing is necessary to achieve maximal species-level resolution for 16S rRNA gene-based microbiome studies of the human oral and respiratory tracts, i.e., the aerodigestive tract. Therefore, we used 16S rRNA gene V1-V2 or V1-V3 short-read datasets to assess the performance of eHOMDv15.1 in Table 2.

The eHOMD is a resource for taxonomic assignment of 16S rRNA gene sequences from the entire human aerodigestive tract, as well as the lower respiratory tract. To assess its performance and the value for analysis of datasets from sites throughout the human aerodigestive tract, eHOMDv15.1 was compared with three commonly used 16S rRNA gene databases and consistently performed better than or comparable to these databases (Table 2). For these comparisons, we used blastn to assign taxonomy to three short-read (V1-V2 and V1-V3) and five approximately full-length-clone-library 16S rRNA gene datasets from the human aerodigestive tract that are publicly available. For short-read datasets, we focused on those covering all or part of the V1-V3 region of the 16S rRNA gene for the reasons discussed above. The chosen datasets include samples from children or adults in health and/or disease. The samples in these datasets are from human nostril swabs, nasal lavage fluid, esophageal biopsies, extubated endotracheal tubes, endotracheal tube aspirates, sputa and bronchoalveolar lavage (BAL) fluid. Endotracheal tube sampling may represent both upper and lower respiratory tract microbes and sputum may be contaminated by oral microbes, whereas BAL fluid represents microbes present in the lower respiratory tract. Therefore, these provide broad representation for bacterial microbiota of the human aerodigestive tract, as well as the human lower respiratory tract (Table 2). The composition of the bacterial microbiota from the nasal passages varies across the span of human life and eHOMD captures this variability. The performance of eHOMDv15.1 in Table 2 establishes it as a resource for microbiome studies of all body sites within the human respiratory and upper digestive tracts.

The eHOMDv15.1 performed very well for nostril samples (Tables 1 and 2), which are a type of skin microbiome sample since the nostrils open onto the skin-covered surface of the nasal vestibules. In various embodiments, the eHOMD may also perform well for other skin sites. To test this hypothesis, we used eHOMDv15.04 to perform blastn for taxonomic assignment of 16S rRNA gene reads from the complete set of clones from multiple nonnasal skin sites generated by Segre, Kong and colleagues (SKs dataset). As shown in Table 4, eHOMDv15.04 performed very well for oily skin sites (alar crease, external auditory canal, back, glabella, manubrium, retroauricular crease and occiput) and the nostrils (nares), identifying >88% of the clones, which was more than the other databases for six of these eight sites. Either SILVA128 or eHOMDv15.04 consistently identified the most clones for each skin site to species level (98.5% identity and 98% coverage); eHOMDv15.04 is almost identical to the released eHOMDv15.1. In contrast, eHOMDv15.04 performed less well than SILVA128 for the majority of the moist skin sites (Table 4), e.g., the axillary vault (arm pit). A review of the details of these results revealed that a further expansion comparable to what we did to go from an oral-focused to an aerodigestive tract-focused database is necessary for eHOMD to include the full diversity of all skin sites.

The eHOMD is a resource for annotated genomes matched to HMTs for use in metagenomic and metatranscriptomic studies. Well-curated and annotated reference genomes correctly named at the species level are a critical resource for mapping metagenomic and metatranscriptomic data to gene and functional information, and for identifying species-level activity within the microbiome. There are currently >160,000 microbial genomic sequences deposited to GenBank; however, many of these genomes remain poorly or not-yet annotated or lack species-level taxonomy assignment, thus limiting the functional interpretation of metagenomic/metatranscriptomic studies to the genus level. Therefore, as an ongoing process, one goal of the eHOMD is to provide correctly named, curated and annotated genomes for all HMTs. In generating eHOMDv15.1, we determined the species-level assignment for 117 genomes in GenBank that were previously identified only to the genus level and which matched to 25 eHOMD taxa (Supplemental Data S1C and S1D). For each of these genomes, the phylogenetic relationship to the assigned HMT was verified by both phylogenetic analysis using 16S rRNA gene sequences (Supplemental Data S1C) and by phylogenomic analysis using a set of core proteins and PhyloPhlAn (41) (Supplemental Data S1D). To date, 85% (475) of the cultivated taxa (and 62% of all taxa) included in eHOMD have at least one sequenced genome.

The eHOMD is a resource for species-level assignment to the outputs of high-resolution 16S rRNA gene analysis algorithms. Algorithms, such as DADA2 and MED, permit high-resolution parsing of 16S rRNA gene short-read sequences. Moreover, the RDP naïve Bayesian Classifier is an effective tool for assigning taxonomy to 16S rRNA gene sequences, both full length and short reads, when coupled with a robust, well-curated training set. Together these tools permit species-level analysis of short-read 16S rRNA gene datasets. Because the V1-V3 region is the most informative short-read fragment for most of the common bacteria of the aerodigestive tract, we generated a training set for the V1-V3 region of the 16S rRNA gene that includes all taxa represented in the eHOMD, which is described elsewhere. In our training set, we grouped taxa that were indistinguishable based on the sequence of their V1-V3 region together as supraspecies to preserve subgenus-level resolution, e.g., Staphylococcus capitis_caprae.

Advantages and limitations of the eHOMD. The eHOMD has advantages and limitations when compared to other 16S rRNA gene databases, such as RDP, NCBI, SILVA and Greengenes. Its primary distinction is that eHOMD is dedicated to providing taxonomic, genomic, bibliographic and other information specifically for the approximately 800 microbial taxa found in the human aerodigestive tract (summarized in Table 5). Here, we highlight five advantages of eHOMD. First, the eHOMD is based on extensively curated 16S rRNA reference sets (eHOMDrefs) and a taxonomy that uses phylogenetic position in 16S rRNA-based trees rather than a taxon's currently assigned, or misassigned, taxonomic name. For example, the genus “Eubacteria” in the phylum Firmicutes includes members that should be divided into multiple genera in seven different families. In eHOMD, members of the “Eubacteria” are placed in their phylogenetically appropriate family, e.g., Peptostreptococcaceae, rather than incorrectly into the family Eubacteriaceae. Appropriate taxonomy files are readily available from eHOMD for mothur and other programs. Second, because eHOMD includes a provisional species-level naming scheme, sequences that can only be assigned genus-level taxonomy in other databases are resolved to species level via an HMT number. This enhances the ability to identify and learn about taxa that currently lack full identification and naming. Importantly, the HMT number is stable, i.e., it stays constant even as a taxon is named or the name is changed. This facilitates tracking knowledge of a specific taxon over time and between different studies. Third, in eHOMD, for the 475 taxa with at least one sequenced genome, genomes can be viewed graphically in the dynamic JBrowse genome web viewer or searched using blastn, blastp, blastx, tblastn or tblastx. For taxa lacking accessible genomic sequences the available 16S rRNA sequences are included. Many genomes of aerodigestive tract organisms are in the whole-genome shotgun contigs (wgs) section of NCBI and are visible by blast search only through wgs provided that one knows the genome and can provide the BioProjectID or WGS Project ID. At eHOMD, one can readily compare dozens to over a hundred genomes for some taxa to begin to understand the pangenome of aerodigestive tract microbes. Fourth, we have also complied proteome sequence sets for genome-sequenced taxa enabling proteomics and mass spectra searches on a dataset limited to proteins from 2,000 relevant genomes. Fifth, for analysis of aerodigestive track 16S rRNA gene datasets, eHOMD is a focused collection and, therefore, smaller in size. This results in increased computational efficiency compared to the other databases. eHOMD performed a blastn of ten 16S rRNA gene full length reads in 0.277 seconds, while the same analysis with the NCBI 16 database took 3.647 seconds and RDP and SILVA needed more than 1 minute (see Supplementary Methods).

In terms of limitations, the taxa included in the eHOMD, the 16S rRNA reference sequences and genomes are not appropriate for samples from 1) human body sites outside of the aerodigestive and respiratory tracts, 2) nonhuman hosts or 3) the environment. In contrast, RDP, SILVA and Greengenes are curated 16S rRNA databases inclusive of all sources and environments. Whereas, the NCBI 16S database is a curated set of sequences for bacterial and archaeal named species only (aka RefSeqs) that is frequently updated. Finally, the NCBI nucleotide database (nr/nt) includes the largest set of 16S rRNA sequences available; however, the vast majority have no taxonomic attribution and are listed as simply “uncultured bacterium clone.” Thus, RDP, SILVA, NCBI, Greengenes and other similar general databases have advantages for research on microbial communities outside the human respiratory and upper digestive tracts, whereas eHOMD is preferred for the microbiomes of the human upper digestive and respiratory tracts.

II. The eHOMD Revealed Previously Unknown Properties of the Human Nasal Microbiome.

To date the human nasal microbiome has mostly been characterized at the genus level. For example, the Human Microbiome Project (HMP) characterized the bacterial community in the adult nostrils (nares) to the genus level using 16S rRNA sequences. However, the human nasal passages can host a number of genera that include both common commensals and important bacterial pathogens, e.g., Staphylococcus, Streptococcus, Haemophilus, Moraxella and Neisseria. Thus, species-level nasal microbiome studies are needed from both a clinical and ecological perspective. Therefore, to further our understanding of the adult nostril microbiome, we used MED, the RDP classifier and our eHOMD V1-V3 training set to reanalyze a subset of the HMP nares V1-V3 16S rRNA dataset consisting of one sample each from 210 adults (see Methods). Henceforth, we refer to this subset as the HMP nares V1-V3 dataset. This resulted in species/supraspecies-level taxonomic assignment for 95% of the sequences and revealed new insights into the adult nostril microbiome, which are described below.

A small number of cultivated species account for the majority of the adult nostril microbiome. Genus-level information from the HMP corroborates data from smaller cohorts showing the nostril microbiome has a very uneven distribution both overall and per person. In our reanalysis, 10 genera accounted for 95% of the total reads from 210 adults (see Methods), with the remaining genera each present at very low relative abundance and prevalence (FIG. 2A and Table S4A). Moreover, for the majority of participants, 5 or fewer genera constituted 90% of the sequences in their sample (FIG. 2B). This uneven distribution characterized by the numeric dominance of a small number of taxa was even more striking at the species level. We found that the 6 most relatively abundant species made up 72% of the total sequences, and the top 5 each had a prevalence of ≥81% (FIG. 2C and Table S4B). Moreover, between 2 and 10 species accounted for 90% of the sequences in 94% of the participants (FIG. 2D). Also, just 19 species/supraspecies-level taxa constituted 90% of the total 16S rRNA gene sequences from all 210 participants (Table S4B), and one of these belonged to an as-yet-uncultivated genus, as described below. The implication of these findings is that in vitro consortia consisting of small numbers of species can effectively represent the natural nasal community, facilitating functional studies of the nostril microbiome.

Identification of two previously unrecognized common nasal bacterial taxa. Reanalysis of both the HMP nares V1-V3 dataset and the SKn 16S rRNA gene clone dataset revealed two previously unrecognized taxa are common in the nostril microbiome: Lawsonella clevelandensis and an unnamed Neisseriaceae [G-1] bacterium, to which we assigned the provisional name Neisseriaceae [G-1] bacterium HMT-174. These are discussed in further detail below.

The human nasal passages are the primary habitat for a subset of bacterial species. The topologically external surfaces of the human body are the primary habitat for a number of bacterial taxa, which are often present at both high relative abundance and high prevalence in the human microbiome. In generating the eHOMDv15.1, we hypothesized that comparing the relative abundance of sequences identified to species or supraspecies level in the SKn clones and the SKs clones (nonnasal skin sites) would permit putative identification of the primary body-site habitat for a subset of nostril-associated bacteria. Based on criteria described in the methods, we putatively identified 13 species as having the nostrils and 1 species as having skin as their primary habitat (Table S5). Online at http://ehomd.org/index.php?name=HOMD the primary body site for each taxon is denoted as oral, nasal, skin, vaginal or unassigned. Definitive identification of the primary habitat of all human-associated bacteria will require species-level identification of bacteria at each distinct habitat across the surfaces of the human body from a cohort of individuals. This would enable a more complete version of the type of comparison performed here.

Members of the genus Corynebacterium (phylum Actinobacteria) are common in human nasal, skin and oral microbiomes but their species-level distribution across these body sites remains less clear. Our analysis of the SKns clones identified three Corynebacterium as primarily located in the nostrils compared to the other skin sites: C. propinquum, C. pseudodiphtheriticum and C. accolens (Table S5). In the species-level reanalysis of the HMP nares V1-V3 dataset, these were among the top five Corynebacterium species/supraspecies by rank order abundance of sequences (Table S4B). In this reanalysis, Corynebacterium tuberculostearicum accounted for the fourth largest number of sequences; however, in the SKns clones it was not disproportionately present in the nostrils. Therefore, although common in the nostrils, we did not consider the nostrils the primary habitat for C. tuberculostearicum, in contrast to C. propinquum, C. pseudodiphtheriticum and C. accolens.

The human skin and nostrils are primary habitats for Lawsonella clevelandensis. In 2016, Lawsonella clevelandensis was described as a novel genus and species within the suborder Corynebacterineae (phylum Actinobacteria); genomes for two isolates are available. It was initially isolated from several human abscesses, mostly from immunocompromised hosts, but its natural habitat was unknown. This led to speculation L. clevelandensis might either be a member of the human microbiome or an environmental microbe with the capacity for opportunistic infection. Our results indicate that L. clevelandensis is a common member of the bacterial microbiome of some oily skin sites and the nostrils of humans (Table S5). Indeed, in the SKn clones, we detected L. clevelandensis as the 11^{th} most abundant taxon. Validating the SKn data in our reanalysis of the HMP nares V1-V3 dataset from 210 participants, we found that L. clevelandensis was the 5th most abundant species overall with a prevalence of 86% (Table S4B). In the nostrils of individual HMP participants, L. clevelandensis had an average relative abundance of 5.7% and a median relative abundance of 2.6% (range 0 to 42.9%). L. clevelandensis is recently reported to be present on skin. Our reanalysis of the SKns clones indicated that of these body sites the primary habitat for L. clevelandensis is oily skin sites, in particular the alar crease, glabella and occiput where it accounts for higher relative abundance than in the nostrils (Table S5). Virtually nothing is known about the role of L. clevelandensis in the human microbiome. By report, it grows best under anaerobic conditions (<1% O₂) and cells are a mixture of pleomorphic cocci and bacilli that stain gram-variable to gram-positive and partially acid fast. Based on its 16S rRNA gene sequence, L. clevelandensis is most closely related to the genus Dietzia, which includes mostly environmental species. Within its suborder Corynebacterineae are other human associated genera, including Corynebacterium, which is commonly found on oral, nasal and skin surfaces, and Mycobacterium. Our analyses demonstrate L. clevelandensis is a common member of the human skin and nasal microbiomes, opening up opportunities for future research on its ecology and its functions with respect to humans.

The majority of the bacteria detected in our reanalysis of the human nasal passages are cultivated. Using blastn to compare the 16S rRNA gene SKn clones with eHOMDv15.1, we found that 93.1% of these sequences from adult nostrils can be assigned to cultivated named species, 2.1% to cultivated unnamed taxa, and 4.7% to uncultivated unnamed taxa. In terms of the total number of species-level taxa represented by the SKn clones, rather than the total number of sequences, 70.1% matched to cultivated named taxa, 14.4% to cultivated unnamed taxa, and 15.5% uncultivated unnamed taxa. Similarly, in the HMP nares V1-V3 dataset from 210 participants (see below), 91.1% of sequences represented cultivated named bacterial species. Thus, the bacterial microbiota of the nasal passages is numerically dominated by cultivated bacteria. In contrast, approximately 30% of the oral microbiota (ehomd.org) and a larger, but not precisely defined, fraction of the intestinal microbiota is currently uncultivated. The ability to cultivate the majority of species detected in the nasal microbiota is an advantage when studying the functions of members of the nasal microbiome.

One common nasal taxon remains to be cultivated. In exploring the SKn dataset to generate eHOMD, we realized that the 12th most abundant clone in the SKn dataset lacked genus-level assignment. To ensure this was not just a common chimera, we broke the sequence up into thirds and fifths and subjected each fragment to blastn against eHOMD and GenBank. The fragments hit only our reference sequences and were distant to other sequences across the entire length. Therefore, this clone represents an unnamed and apparently uncultivated Neisseriaceae bacterial taxon to which we have assigned the provisional name Neisseriaceae [G-1] bacterium HMT-174 ([G-1] to designate unnamed genus 1). Its provisional naming facilitates recognition of this bacterium in other datasets and its future study. In our reanalysis of the HMP nares V1-V3 dataset, Neisseriaceae [G-1] bacterium HMT-174 was the 10th most abundant species overall with a prevalence of 35%. In individual participants, it had an average relative abundance of 1.3% and a median relative abundance of 0 (range 0 to 38.4%). Blastn analysis of our reference sequence for Neisseriaceae [G-1] bacterium HMT-174 against the 16S ribosomal RNA sequences database at NCBI gave matches of 90% to 92% similarity to members of the family Neisseriaceae and matches to the neighboring family Chromobacteriaceae at 88% to 89%. A phylogenetic tree of taxon HMT-174 with members of these two families was more instructive since it clearly placed taxon HMT-174 as a deeply branching, but monophyletic, member of the Neisseriaceae family with the closest named taxa being Snodgrassella alvi (NR_118404) at 92% similarity and Vitreoscilla stercoraria (NR_0258994) at 91% similarity, and the main cluster of Neisseriaceae at or below 92% similar (Supplemental Data S1E). The main cluster of genera in a tree of the family Neisseriaceae includes Neisseria, Alysiella, Bergeriella, Conchiformibius, Eikenella, Kingella and other mammalian host-associated taxa. There is a separate clade of the insect associated genera Snodgrassella and Stenoxybacter, whereas Vitreoscilla is from cow dung and forms its own clade. Recognition of the as-yet-uncultivated Neisseriaceae [G-1] bacterium HMT-174 as a common member of the adult nostril microbiome supports future research to cultivate and characterize this bacterium. Neisseriaceae [G-1] bacterium HMT-327 is another uncultivated nasal taxon, likely from the same unnamed genus, and the 20th (HMP) and 46^{th} (SKn) most common nasal organism in the two datasets we reanalyzed. There are several additional uncultured nasal bacteria in eHOMD, highlighting the need for sophisticated cultivation studies even in the era of NGS studies. Having 16S rRNA reference sequences tied to the provisional taxonomic scheme in eHOMD allows targeted efforts to culture the previously uncultivated based on precise 16S rRNA identification methods.

No species are differentially abundant with respect to either Neisseriaceae [G-1] bacterium HMT-174 or L. clevelandensis. There is a lack of knowledge about potential relationships between the two newly recognized members of the nostril microbiome, L. clevelandensis and Neisseriaceae [G-1] bacterium HMT-174, and other known members of the nostril microbiome. Therefore, we performed Analysis of Composition of Microbiomes, aka ANCOM, on samples grouped based on the presence or absence of sequences of each of these two taxa of interest in search of species displaying differential relative abundance based on either one (FIG. 3A). For Neisseriaceae [G-1] bacterium HMT-174, this was targeted at identifying potential growth partners for this as-yet-uncultivated bacterium. However, ANCOM detected only the group-specific taxon in each case and did not reveal any other species with differential relative abundance with respect to either Neisseriaceae [G-1] bacterium HMT-174 (FIG. 3B) or L. clevelandensis (FIG. 3C).

Several common species of nasal bacteria are more abundant when S. aureus is absent. Finally, as proof of principle that eHOMD enhances the clinical relevance of 16S rRNA gene-based microbiome studies, we turned our attention to S. aureus, which is both a common member of the nasal microbiome and an important human pathogen, with >10,000 attributable deaths/year in the U.S. The genus Staphylococcus includes many human commensals hence the clinical importance of distinguishing aureus from non-aureus species. In our reanalysis of the HMP nares V1-V3 dataset, S. aureus sequences accounted for 3.9% of the total sequences with a prevalence of 34% (72 of the 210 participants), consistent with it being common in the nasal microbiome. S. aureus nostril colonization is a risk factor for invasive infection at distant body sites. Therefore, in the absence of an effective vaccine, there is increasing interest in identifying members of the nostril and skin microbiome that might play a role in colonization resistance to S. aureus, e.g., Although differential relative abundance does not indicate causation, identifying such relationships at the species level in a cohort the size of the HMP can arbitrate variations among findings in smaller cohorts and generate new hypotheses for future testing. Therefore, we used ANCOM to identify taxa displaying differential relative abundance in HMP nostril samples in which 16S rRNA gene sequences corresponding to S. aureus were absent or present. In this HMP cohort of 210 adults, two Corynebacterium species/supraspecies—accolens and accolens_macginleyi_tuberculostearicum—showed positive differential abundance in the absence of S. aureus nostril colonization (FIG. 3D, panels i and ii). These two were among the nine most abundant species in the cohort overall (FIG. 2C and Table S4B). As previously reviewed, there is variability between studies with smaller cohorts with respect to the reported correlations between S. aureus and specific Corynebacterium species in the nostril microbiome; this variability might relate to strain-level differences and/or to the small cohort sizes. D. pigrum also showed a positive differential abundance in the absence of S. aureus (FIG. 3D, panel iii). This is consistent with observations from Liu, Andersen and colleagues that high-levels of D. pigrum are the strongest predictor of absence of S. aureus nostril colonization in 89 older adult Danish twin pairs. In our reanalysis of the HMP nares V1-V3 dataset, D. pigrum was the 6th most abundant species overall with a prevalence of 41% (FIG. 2C and Table S4B). There were no species, other than the group-specific taxon S. aureus, with positive differential abundance when S. aureus was present (FIG. 3D, panel iv).

Summary. As demonstrated here, the eHOMD (ehomd.org) is a comprehensive well-curated online database for the bacterial microbiome of the entire aerodigestive tract enabling species/supraspecies-level taxonomic assignment to full-length and V1-V3 16S rRNA gene sequences and including correctly assigned, annotated available genomes. In generating the eHOMD, we identified two previously unrecognized common members of the adult human nostril microbiome, opening up new avenues for future research. As illustrated using the adult nostril microbiome, eHOMD can be leveraged for species-level analyses of the relationship between members of the aerodigestive tract microbiome, enhancing the clinical relevance of studies and generating new hypotheses about interspecies interactions and the functions of microbes within the human microbiome. The eHOMD provides a broad range of microbial researchers, from basic to clinical, a resource for exploring the microbial communities that inhabit the human respiratory and upper digestive tracts in health and disease.

### MATERIALS AND METHODS

Generating the provisional eHOMDv15.01 by adding bacterial species from culture-dependent studies. To identify candidate Human Microbial Taxa (cHMTs), we reviewed two studies that included cultivation of swabs taken from along the nasal passages in both health and chronic rhinosinusitis (CRS) and one study of mucosal swabs and nasal washes only in health. We also reviewed a culture-dependent study of anaerobic bacteria isolated from cystic fibrosis (CF) sputa to identify anaerobes that might be present in the nasal passages/sinuses in CF. Using this approach, we identified 162 cHMTs, of which 65 were present in HOMDv14.51 and 97 were not (FIG. 1A and Table S1A). For each of these 97 named species, we downloaded at least one 16S rRNA gene RefSeq from NCBI 16S (via a search of BioProjects 33175 and 33317) and assembled these into a reference database for blast. We then queried this via blastn with the SKn dataset to determine which of the 97 cHMTs were either residents or very common transients of the nasal passages (FIG. 1A). We identified 30 cHMTs that were represented by ≥10 sequences in the SKn dataset with a match at ≥98.5% identity. We added these 30 candidate taxa, represented by 31 16S rRNA gene reference sequences for eHOMD (eHOMDrefs), as permanent HMTs to the HOMDv14.51 alignment to generate the eHOMDv15.01 (FIG. 1A and Table S6A). Of note, with the addition of nonoral taxa, we have replaced the old provisional taxonomy prefix of Human Oral Taxon (HOT) with Human Microbial Taxon (HMT), which is applied to all taxa in the eHOMD.

Generating the provisional eHOMDv15.02 by identifying additional HMTs from a dataset of 16S rRNA gene clones from human nostrils. For the second step on the HOMD expansion, we focused on obtaining new eHOMDrefs from the SKn dataset (i.e., the 44,374 16S rRNA gene clones from nostril (anterior nares)). We used blastn to query the SKn clones versus the provisional database eHOMDv15.01. Of the nostril-derived 16S rRNA gene clones, 37,716 of 44,374 matched reference sequences in eHOMDv15.01 at ≥98.5% identity (FIG. 1B) and 6163 matched to eHOMDv15.01 at ≤98% (FIG. 1C). The SKn clones that matched eHOMDv15.01 at ≥98.5% could be considered already identified by eHOMDv15.01. Nevertheless, these already identified clones were used as query to perform blastn versus the NCBI 16S database to identify other NCBI RefSeqs that might match these clones with a better identity. We compared the blastn results against eHOMDv15.01 and NCBI 16S and if the match was substantially better to a high-quality sequence (close to full length and without unresolved nucleotides) from the NCBI 16S database then that one was considered for addition to the database. Using this approach, we identified two new HMTs (represented by one eHOMDref each) and five new eHOMDrefs for taxa present in eHOMDv14.51 that improved capture of sequences to these taxa (FIG. 1B and Table S6A). For the 6163 SKn clones that matched to eHOMDv15.01 at <98%, we performed clustering at ≥98.5% identity across 99% coverage and inferred an approximately maximum-likelihood phylogenetic tree (FIG. 1C and Supplemental Methods). If a cluster (an M-OTU) had ≥10 clone sequences (30 out of 32), then we chose representative sequence(s) from that cluster based on a visual assessment of the cluster alignment. Each representative sequence was then queried against the NCBI nr/nt database to identify either the best high-quality, named species-level match or, lacking this, the longest high-quality clone sequence to use as the eHOMDref. Clones lacking a named match were assigned a genus name based on their position in the tree and an HMT number, which serves as a provisional name. The cluster representative sequence(s) plus any potentially superior reference sequences from the NCBI nr/nt database were finally added to the eHOMDv15.01 alignment to create the eHOMDv15.02. Using this approach, we identified and added 28 new HMTs, represented in total by 38 eHOMDrefs (FIG. 1C and Table S6A). Of note, we set aside the 1.1% (495 of 44,374) of SKn clones that matched at between 98 and 98.5% identify, to avoid calling a taxon where no new taxon existed in the tree-based analysis of sequences that matched at <98%.

Generating the provisional eHOMDv15.03 by identifying additional candidate taxa from culture-independent studies of aerodigestive tract microbiomes. To further improve the performance of the evolving eHOMD, we took all of the SKn dataset clones that matched eHOMDv15.02 at <98.5% identity, clustered these at ≥98.5% identity across a coverage of 99% and inferred an approximately maximum-likelihood phylogenetic tree (Supplemental Methods). Subsequent evaluation of this tree (see previous section) identified two more HMTs (represented in total by 3 eHOMDrefs) and one new eHOMDref for a taxon already in the database for addition to eHOMDv15.03 (FIG. 1D and Table S6A). To identify additional taxa that are resident to sites in the aerodigestive tract beyond the mouth and that are not represented by enough clones in the SKn dataset to meet our criteria, we iteratively evaluated the performance of eHOMDv15.02 with 5 other 16S rRNA gene datasets from aerodigestive tract sites outside the mouth (FIG. 1E). We used the following criteria to select these datasets to assay for the performance of eHOMDv15.02 as a reference database for the aerodigestive tract across the span of human life in health and disease: (1) all sequences covered at least variable regions 1 and 2 (V1-V2), because for many bacteria resident in the aerodigestive tract V1-V2/V1-V3 includes sufficient sequence variability to get towards species-level assignment (Table 3); and (2) the raw sequence data was either publicly available or readily supplied by the authors upon request. This approach yielded a representative set of datasets (Table SIC). Additional information on how we obtained and prepared each dataset for use is in Supplemental Methods. For each dataset from Table S1C, we separately performed a blastn against eHOMDv15.02 and filtered the results to identify the percent of reads matching at ≥98.5% identity (FIG. 1E). To compare the performance of eHOMDv15.02 with other commonly used 16S rRNA gene databases, we also performed a blastn against NCBI 16S, RDP16 and SILVA128 databases using the same filter as with eHOMDv15.02 for each dataset (Table S1C). If one of these other databases captured more sequences than eHOMDv15.02 at ≥98.5% identity, we then identified the reference sequence in the outperforming database that was capturing those sequences and evaluated it for inclusion in eHOMD. Based on this comparative approach, we added three new HMTs (represented by one eHOMDref each) plus five new eHOMDrefs for taxa already present in eHOMDv15.02 to the provisional database to create eHOMDv15.03 (FIG. 1E and Table S6A).

Generating the provisional eHOMDv15.04 by identifying additional candidate taxa from a dataset of 16S rRNA gene clones from human skin. Having established that eHOMDv15.03 serves as an excellent 16S rRNA gene database for the aerodigestive tract microbiome in health and disease, we were curious as to how it would perform when evaluating 16S rRNA gene clone libraries from skin sites other than the nostrils. In humans, the area just inside the nostrils, which are the openings into the nasal passages, is the skin-covered-surface of the nasal vestibule. Prior studies have demonstrated that the bacterial microbiota of the skin of the nasal vestibule (aka nostrils or nares) is distinctive and most similar to other moist skin sites. To test how well eHOMDv15.03 performed as a database for skin microbiota in general, we executed a blastn using 16S rRNA gene clones from all of the nonnasal skin sites included in the Segre-Kong dataset (SKs) to assess the percentage of total sequences captured at ≥98.5% identity over ≥98% coverage. Only 81.7% of the SKs clones were identified with eHOMDv15.03, whereas 95% of the SKn clones were identified (Table SIB). We took the unidentified SKs sequences and did blastn versus the SILVA128 database with the same filtering criteria. To generate eHOMDv15.04, we first added the top 10 species from the SKs dataset that did not match to eHOMDv15.03, all of which had >350 reads in SKs (FIG. 1D and Table S6A). Of note, for two of the skin-covered body sites a single taxon accounted for the majority of reads that were unassigned with eHOMDv15.03: Staphylococcus auricularis from the external auditory canal and Corynebacterium massiliense from the umbilicus. Addition of these two considerably improved the performance of eHOMD for their respective body site. Next, we revisited the original list of 97 cHMTs and identified 4 species that are present in ≥3 of the 34 subjects (Table S1A), that had ≥30 reads in the SKs dataset and that matched to SILVA128 but not to eHOMDv15.03. These we added to generate eHOMDv15.04 (FIGS. 1A-1D and Table S6A).

Establishing eHOMD reference sequences and final updates to generate eHOMDv15.1. Each eHOMD reference sequence (eHOMDref) is a manually corrected representative sequence with a unique alphanumeric identifier that starts with its three-digit HMT #; each is associated with the original NCBI accession # of the candidate sequence. For each candidate 16S rRNA gene reference sequence selected, a blastn was performed against the NCBI nr/nt database and filtered for matches at ≥98.5% identity to identify additional sequences for comparison in an alignment, which was used to either manually correct the original candidate sequence or select a superior candidate from within the alignment. Manual correction included correction of all ambiguous nucleotides, any likely sequencing miscalls/errors and addition of consensus sequence at the 5′/3′ ends to achieve uniform length. All ambiguous nucleotides from earlier versions were corrected in the transition from HOMDv15.04 to eHOMDv15.1 because ambiguous bases, such as “R” and “Y”, are always counted as mismatches against a nonambiguous base. Also, in preparing v15.1, nomenclature for Streptococcus species was updated in accordance with and genus names were updated for species that were formerly part of the Propionibacterium genus. Cutibacterium is the new genus name for the formerly cutaneous Propionibacterium species. In addition to the 79 taxa added in the expansion from HOMDv14.51 to eHOMDv15.04 (Table S6A), 4 oral taxa were added to the final eHOMDv15.1: Fusobacterium hwasookii HMT-953, Saccharibacteria (TM7) bacterium HMT-954, Saccharibacteria (TM7) bacterium HMT-955 and Neisseria cinerea HMT-956. Also, Neisseria pharyngis HMT-729 was deleted because it is not validly named and is part of the N. sica-N. mucosa-N. flava complex.

Identification of taxa with a preference for the human nasal habitat. We assigned 13 taxa as having the nostrils as their preferred body site habitat. To achieve this, we first performed the following steps as illustrated in Table S5. 1) We performed blastn of SKn and SKs versus eHOMDv15.04 and used the first hit based on e-value to assign putative taxonomy to each clone; 2) used these names to generate a count table of taxa and body sites; 3) normalized the total number of clones per body site to 20,000 each for comparisons (columns B to V); 4) for each taxon, used the total number of clones across all body sites as the denominator (column W) to calculate the % of that clone present at each specific body site (columns Z to AT); 5) calculated the ratio of the % of each taxon in the nostrils to the expected % if that taxon was evenly distributed across all 21 body sites in the SKns clone dataset (column Y); and 6) sorted all taxa in Table S5 by the rank abundance among the nostril clones (column X). Finally, of these top 20, we assigned nasal as the preferred body site to those that were elevated ≥2x in the nostrils versus what would be expected if evenly distributed across all the skin sites (column Y). This conservative approach established a lower bound for the eHOMD taxa that have the nasal passages as their preferred habitat. The SKn dataset includes samples from children and adults in health and disease. In contrast, the HMP nares V1-V3 data are from adults 18 to 40 years of age in health only. Of the species classified as nasal in eHOMDv15.01, 8 of the 13 are in the top 19 most abundant species from the 210-person HMP nares V1-V3 dataset.

Reanalysis of the HMP nares V1-V3 dataset to species level. We aligned the 2,338,563 chimera-cleaned reads present in the HMPnV1-V3 (see Suppl. Methods) in QIIME 1 (align_seqs.py with default method; PyNAST), using eHOMDv15.04 as reference database and trimmed for MED using “o-trim-uninformative-columns-from-alignment” and “o-smart-trim” scripts. 2,203,471 reads (94.2% of starting) were recovered after the alignment and trimming steps. After these initial cleaning steps, samples were selected such that only those with more than 1000 reads were retained and each subject was represented by only one sample. For subjects with more than one sample in the total HMP nares V1-V3 data, we selected for use the one with more reads after the cleaning steps to avoid bias. Thus, what we refer to as the HMP nares V1-V3 dataset included 1,627,514 high quality sequences representing 210 subjects. We analyzed this dataset using MED with minimum substantive abundance of an oligotype (−M) equal to 4 and maximum variation allowed in each node (−V) equal to 12 nt, which equals 2.5% of the 820-nucleotide length of the trimmed alignment. Of the 1,627,514 sequences, 89.9% (1,462,437) passed the −M and −V filtering and are represented in the MED output. Oligotypes were assigned taxonomy in R with the dada2::assignTaxonomy( ) function (an implementation of the RDP naive Bayesian classifier algorithm with a kmer size of 8 and a bootstrap of 100) using the eHOMDv15.1 V1-V3 Training Set (version 1). We then collapsed oligotypes within the same species/supraspecies yielding the data shown in Table S7. The count data in Table S7 was converted to relative abundance by sample at the species/supraspecies level to generate an input table for ANCOM including all identified taxa (i.e., we did not remove taxa with low relative abundance). ANCOM (version 1.1.3) was performed using presence or absence of Neisseriaceae [G-1] bacterium HMT-174, L. clevelandensis or S. aureus as group definers. ANCOM default parameters were used (sig=0.05, tau=0.02, theta=0.1, repeated=FALSE) except that we performed a correction for multiple comparisons (multcorr=2) instead of using the default no correction (multcorr=3).

Recruitment of genomes matching HMTs to eHOMD and assignment of species-level names to genomes previously named only at then genus level. Genomic sequences were downloaded from the NCBI FTP site (ftp://ftp.ncbi.nlm.nih.gov/genomes). Genome information, e.g., genus, species and strain name were obtained from a summary file listed on the FTP site in July 2018: ftp://ftp.ncbi.nlm.nih.gov/genomes/ASSEMBLY_REPORTS/assembly_summary_genbank.txt. To recruit genomes for provisionally named eHOMD taxa (HMTs), genomic sequences from the same genus were targeted. For 6 genera present in eHOMD, we downloaded and analyzed 130 genomic sequences from GenBank that were taxonomically assigned only to the genus level (i.e., with “sp.” in the species annotation) because some of these might belong to a HMT. To determine the closest HMT for each of these genomes, the 16S rRNA genes were extracted from each genome and were blastn-searched against the eHOMDv15.1 reference sequences. Of the 130 genomes tested, we excluded 13 that had <98% sequence identity to any of the eHOMDrefs. The remaining 117 genomes fell within a total of 25 eHOMD taxa at a percent identity ≥98.5% to one of the eHOMDrefs (Table S6B). To validate the phylogenetic relatedness of these genomes to HMTs, the extracted 16S rRNA gene sequences were then aligned with the eHOMDrefs using the MAFFT software (V7.407) and a phylogenetic tree was generated using FastTree (Version 2.1.10.Dbl) with the default Jukes-Cantor+CAT model for tree inference (Supplemental Data S1C). The relationship of these genomes to eHOMD taxa was further confirmed by performing phylogenomic analysis in which all the proteins sequences of these genomes were collected and analyzed using PhyloPhlAn, which infers a phylogenomic tree based on the most conserved 400 bacterial protein sequences (Supplemental Data S1D). These 117 genomes were then added to the eHOMDv15.1 as reference genomes. At least one genome from each taxon is dynamically annotated against a frequently updated NCBI nonredundant protein database so that potential functions may be assigned to hypothetical proteins due to matches to newly added proteins with functional annotation in NCBI nr database.

Supplemental Methods

In exemplary experiments, the following aerodigestive tract microbiome datasets were used. Segre, Kong and colleagues have deposited close-to-full-length 16S RNA gene sequences from clone libraries collected from different skin sites, including the nostrils (nares) at NCBI under BioProjects PRJNA46333 and PRJNA30125 (1-6). A total of 413,606 sequences were downloaded from these BioProjects on May 11, 2017. The sequences were screened for bacterial 16S rRNA gene sequences only and parsed into two datasets: the SK nostril dataset (SKn), which includes 44,374 sequences from nostril samples with a mean length of 1354 bp (min. 1233, max. 1401); and the SK skin dataset (SKs), which includes 362,313 sequences with a mean length of 1356 bp (min. 1161, max. 1410). The SKs dataset includes 16S rRNA clone sequences derived from 20 non-nasal skin sites, including the alar crease, antecubital fossa, axillary vault, back, buttock, elbow, external auditory canal, glabella, gluteal crease, hypothenar palm, inguinal crease, interdigital web space, manubrium, occiput, plantar heel, popliteal fossa, retroauricular crease, toe web space, umbilicus and volar forearm.

The Human Microbiome Project (HMP) Data Coordination Center performed baseline processing and analysis of all 16S rRNA gene variable region sequences generated from >10,000 samples from healthy human subjects (7, 8). Table “HM16STR_healthy.csv” summarizes all the information for the 9811 files included in the dataset (https://www.hmpdacc.org/hmp/HM16STR/healthy). The 586 files labelled “anterior_nares” were downloaded from the corresponding url identified in the same table. The downloaded files contain V1-V3, V3-V5 and V6-V9 data, therefore the reads were filtered based on the primer information recorded in each read header, resulting in a total of U.S. Pat. No. 3,458,862 “anterior_nares” V1-V3 reads corresponding to 363 samples from 227 subjects. The 2,351,347 reads (67.9%) with length 2430 and 5652 bp (the range of the V1-V3 16S rRNA gene region in HOMDv14.51) were selected. After de novo chimera removal with UCHIME in QIIME 1 (9, 10) (identify_chimeric_seqs.py-m usearch61), there were 2,338,563 sequences for use. This dataset, dubbed HMPnV1-V3, was the starting point used to query the performance of the provisional versions of eHOMD and was the input for species-level reanalysis.

Laufer et al. analyzed nostril swabs collected from 108 children ages 6 to 78 months in Philadelphia, Pa. between Dec. 9, 2008 and Jan. 2, 2009 for cultivation of Streptococcus pneumoniae and DNA harvest. Of these, 44% were culture positive for S. pneumoniae and 23% were diagnosed with otitis media. 16S rRNA gene V1-V2 sequences were generated using Roche/454 with primers 27F and 338R. 184,685 sequences were obtained from the authors, of which 94% included sequence matching primer 338R and 1% included sequence matching primer 27F. Therefore, demultiplexing was performed in QIIME 1 (split_libraries.py) filtering reads for those 2250 bp in length, quality score ≥30 and with barcode type hamming_8. Sequences were eliminated from samples for which there was no metadata (n=108 for metadata) leaving 120,963 sequences on which de novo chimera removal was performed with UCHIME in QIIME 1 (identify_chimeric_seqs.py-m usearch61) (9, 10), yielding the 120,274 16S rRNA V1-V2 sequences used here.

Allen et al. collected nasal lavage fluid samples from 10 participants before, during and after experimental nasal inoculation with rhinovirus. 16S rRNA V1-V3 sequences were generated using 454-FLX platform and primers 27F and 534R. 99,095 sequences were obtained from the authors of which 77,322 (78%) passed a length filter of 2300 bp. After de novo chimera removal in with UCHIME in QIIME 1(identify_chimeric_seqs.py−m usearch61) (9, 10), there were 75,310 sequences for use in this study.

Pei et al. (2004) collected distal esophageal biopsies from four participants undergoing esophagogastroduodenoscopy for upper gastrointestinal complaints whose samples showed healthy esophageal tissue without evidence of pathology. From each of these, they generated ten 16s rRNA gene clone libraries from independent amplifications using two different primer pairs: 1) 318 to 1,519 with inosine at ambiguous positions and 2) from 8 to 1513. Pei et al. (2005) also collected esophageal biopsies from 24 patients (9 with normal esophageal mucosa, 12 with gastroesophageal reflux disease (GERD), and 3 with Barrett's esophagus) (14). The Pei et al. 2004-2005 dataset also include all the novel sequences deposited in GenBank from this subsequent study. A total of 7,414 close-to-full-length 16S rRNA gene sequences were downloaded from GenBank (GB: DQ537536.1 to DQ537935.1 and DQ632752.1 to DQ639751.1 (PopSet 109141097), AY212255.1 to AY212264.1 (PopSet 28894245), AY394004.1, AY423746.1, AY423747.1 and AY423748.1).

Harris et al. collected bronchoalveolar lavage fluid from children with cystic fibrosis and generated 16S rRNA clone libraries from these. These 3203 clones were downloaded from GenBank (GB: EU111806.1 to EU112454.1 (PopSet 157058892), DQ188268.1 to DQ188805.1 (PopSet 77819181) and AY805987.1 to AY808002.1 (PopSet 60499797)).

van der Gast et al. generated 16S rRNA gene clone libraries from spontaneously expectorated sputum samples collected from 14 adults with cystic fibrosis. These 2137 clones were downloaded from GenBank (GB: FM995625.1 to FM997761.1).

Flanagan et al. generated 16S rRNA gene clone libraries from daily endotracheal aspirates collected from seven intubated patients. These 3278 clones were downloaded from GenBank (GB: EF508731.1 to EF512008.1).

Perkins et al. collected endotracheal tubes from eight adults with mechanical ventilation to generate 16S rRNA gene clone libraries. These 1263 clones were downloaded from GenBank (GB: FJ557249.1 to FJ558511.1).

In exemplary experiments, the following 16S rRNA gene databases were used. The NCBI 16S Microbial database (NCBI 16S) was downloaded from ftp://ftp.ncbi.nlm.nih.gov/blast/db/ on May 28, 2017 (19). RDP16 (rdp_species_assignment_16.fa.gz) and SILVA128 (silva_species_assignment_v128.fa.gz) files were downloaded from https://benjjneb.github.io/dada2/training.html and converted to BLAST databases using “makeblastdb” from the NCBI blast 2.6.0+ package (https://www.ncbi.nlm.nih.gov/books/NBK279690) (20-22).

Greengenes GOLD was used instead of Greengenes because only 22.6% of 16S rRNA gene sequences in Greengenes had complete taxonomic information to the species level, whereas for 77.4% of the sequences the 7th (species) level was listed simply as “s_”. In contrast, in Greengenes GOLD all sequences included 7 levels of taxonomic information, as needed for species-level identification. The Greengenes GOLD was downloaded from http://greengenes.lbl.gov/Download/Sequence_Data/Fasta_data_files/gold_strains_ggl6S_aligned.fasta.gz. The total number of sequences in the database is 5441 (six of the entries in the fasta file consisted only of a header without data, thus were removed). The aligned fasta file was converted to a nonaligned file by removing all “.” and “-”, and further converted to a BLAST database using “makeblastdb” as above.

In exemplary experiments, 16S rRNA sequences were added to the e HOMD alignment as follows. eHOMD maintains an alignment of all its reference 16S rRNA sequences. This alignment is based on the 16S rRNA secondary structure and is performed manually on a custom sequence editor (written in QuickBasic and available from Floyd E. Dewhirst at fdewhirst{at}forsyth.org). The corresponding alignment, in phylogenetic order, for each release of HOMD/eHOMD can be downloaded at http://www.homd.org/?name=seqDownload&type=R.

In exemplary experiments, sequences are clustered at ≥98.5% and phylogenetic trees are generated as follows. blastn was performed with an all-by-all search of the input sequences (FIGS. 1C and 1D). The blastn results were used to cluster the sequences into operational taxonomic units (OTUs) based on percent sequence identity and alignment coverage. Specifically, all sequences were first sorted by size (seq_sort_len.fasta) in descending order and binned into operational taxonomic units (OTUs) at ≥98.5% identity across ≥99% coverage from longest to shortest sequences. If any subsequent sequence matched a previous sequence at ≥98.5% with coverage of ≥99%, the subsequent sequence was binned together with the previous sequence. If the subsequent sequence did not match any previous sequence, it was placed in new bin (i.e., 98.5% OTU). If the subsequent sequences matched multiple previous sequences that belong to more than one OTU, the subsequent sequence was binned to multiple OTUs, and at the same time, we formed a meta-OTU (M-OTU) linking these OTUs together. Next, sequences were extracted from each M-OTU and saved to individual fasta files. Sequence alignment was performed using software MAFFT (V7.407) for each M-OTU fasta file and constructed phylogenetic trees for each M-OTU. The trees were built using FastTree (v2.1.10.db1), which estimates nucleotide evolution with the Jukes-Cantor model and infers phylogenetic trees based on approximately maximum-likelihood. The trees were organized by using the longest branch as root and ordered from fewest nodes to more subnodes.

Of the 97 cHMTs for addition to HOMD, 82 are present in a nasal culturome of 34 participants (Table 51A, column E), 18 with evidence of chronic nasal inflammation and 16 without evidence of nasal/systemic inflammation, based on swabs taken during nasal surgery from the anterior and posterior nasal vestibule (skin surface inside the nostrils) and the inferior and middle meatuses. Of the other 15 cHMTs we found 7 only in a report of cultivation of intraoperative mucosal swabs from 38 participants with chronic rhinosinusitis (CRS) versus 6 controls; 7 only in sputa from 50 adults with CF; and 1 only in a report of the aerobic bacteria collected via a mucosal swab of the inferior turbinate and via a nasal wash from each of 10 healthy adults.

Ten 16S rRNA gene full length reads were randomly extracted from the SKn dataset for use as query in a blastn vs. different databases. The blast 2.6.0+ command: “blastn −db YOURDATABASEHERE −query YOURQUERYFILEHERE −out OUTPUT.txt −outfmt “10 std qcovs salltitles”−max_target_seqs 1” was run using a single processor thread on a computer with the Intel Xeon CPU (X5675 @ 3.07 GHZ with 24 Gb memory). The Linux shell command “time” was used before the blastn command to record the running time.

Supplemental Tables

Supplemental Table S1: The expanded eHOMDv15.1 was generated by (A) identifying candidate taxa from culture-dependent studies, (B) 16S rRNA gene clones from human nostrils and (C) skin and culture-independent studies of aerodigestive tract microbiomes.

Supplemental Table S2: Comparison of the taxonomic assignment at species-level by blastn of the SKn clones using eHOMDv15.1 vs. SILVA128 revealed a subset of reads that were classified as captured at 98.5% identity and 98% coverage by both databases but (A) had differential species-level assignment, (B) were identified only with SILVA, or (C) were identified only with eHOMDv15.1.

Supplemental Table S3: The subsets of taxa that collapsed into undifferentiated groups at each percent identity threshold (100%, 99.5% and 99%) for the (A-C) V1-V3 and (D-F) V3-V4 regions of the 16S rRNA gene, respectively.

Supplemental Table S4: (A) Genus and (B) species/supraspecies rank order abundance of sequences in the reanalysis of the HMP nares V1-V3 16S rRNA gene dataset.

Supplemental Table S5: Identification of taxa with a preference for the human nasal habitat using the SKn and SKs datasets.

Supplemental Table S6: Summary of additions in the current expansion of HOMD in order to generate eHOMDv15.1, including (A) new eHOMDrefs added to both new and existing HMTs, and (B) newly added genomes.

Supplemental Table S7: Table of counts per sample and taxa in the HMP nares V1-V3 dataset result of the reanalysis at the species/supraspecies level.

Referring now to FIG. 26, a schematic of an exemplary computing node is shown that may be used with the computer vision systems described herein. Computing node 10 is only one example of a suitable computing node and is not intended to suggest any limitation as to the scope of use or functionality of embodiments described herein. Regardless, computing node 10 is capable of being implemented and/or performing any of the functionality set forth hereinabove.

In computing node 10 there is a computer system/server 12, which is operational with numerous other general purpose or special purpose computing system environments or configurations. Examples of well-known computing systems, environments, and/or configurations that may be suitable for use with computer system/server 12 include, but are not limited to, personal computer systems, server computer systems, thin clients, thick clients, handheld or laptop devices, multiprocessor systems, microprocessor-based systems, set top boxes, programmable consumer electronics, network PCs, minicomputer systems, mainframe computer systems, and distributed cloud computing environments that include any of the above systems or devices, and the like.

Computer system/server 12 may be described in the general context of computer system-executable instructions, such as program modules, being executed by a computer system. Generally, program modules may include routines, programs, objects, components, logic, data structures, and so on that perform particular tasks or implement particular abstract data types. Computer system/server 12 may be practiced in distributed cloud computing environments where tasks are performed by remote processing devices that are linked through a communications network. In a distributed cloud computing environment, program modules may be located in both local and remote computer system storage media including memory storage devices.

As shown in FIG. 26, computer system/server 12 in computing node 10 is shown in the form of a general-purpose computing device. The components of computer system/server 12 may include, but are not limited to, one or more processors or processing units 16, a system memory 28, and a bus 18 coupling various system components including system memory 28 to processor 16.

Bus 18 represents one or more of any of several types of bus structures, including a memory bus or memory controller, a peripheral bus, an accelerated graphics port, and a processor or local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus.

Computer system/server 12 typically includes a variety of computer system readable media. Such media may be any available media that is accessible by computer system/server 12, and it includes both volatile and non-volatile media, removable and non-removable media.

System memory 28 can include computer system readable media in the form of volatile memory, such as random access memory (RAM) 30 and/or cache memory 32. Computer system/server 12 may further include other removable/non-removable, volatile/non-volatile computer system storage media. By way of example only, storage system 34 can be provided for reading from and writing to a non-removable, non-volatile magnetic media (not shown and typically called a “hard drive”). Although not shown, a magnetic disk drive for reading from and writing to a removable, non-volatile magnetic disk (e.g., a “floppy disk”), and an optical disk drive for reading from or writing to a removable, non-volatile optical disk such as a CD-ROM, DVD-ROM or other optical media can be provided. In such instances, each can be connected to bus 18 by one or more data media interfaces. As will be further depicted and described below, memory 28 may include at least one program product having a set (e.g., at least one) of program modules that are configured to carry out the functions of embodiments of the disclosure.

Program/utility 40, having a set (at least one) of program modules 42, may be stored in memory 28 by way of example, and not limitation, as well as an operating system, one or more application programs, other program modules, and program data. Each of the operating system, one or more application programs, other program modules, and program data or some combination thereof, may include an implementation of a networking environment. Program modules 42 generally carry out the functions and/or methodologies of embodiments described herein.

Computer system/server 12 may also communicate with one or more external devices 14 such as a keyboard, a pointing device, a display 24, etc.; one or more devices that enable a user to interact with computer system/server 12; and/or any devices (e.g., network card, modem, etc.) that enable computer system/server 12 to communicate with one or more other computing devices. Such communication can occur via Input/Output (I/O) interfaces 22. Still yet, computer system/server 12 can communicate with one or more networks such as a local area network (LAN), a general wide area network (WAN), and/or a public network (e.g., the Internet) via network adapter 20. As depicted, network adapter 20 communicates with the other components of computer system/server 12 via bus 18. It should be understood that although not shown, other hardware and/or software components could be used in conjunction with computer system/server 12. Examples, include, but are not limited to: microcode, device drivers, redundant processing units, external disk drive arrays, RAID systems, tape drives, and data archival storage systems, etc.

In other embodiments, the computer system/server may be connected to one or more cameras (e.g., digital cameras, light-field cameras) or other imaging/sensing devices (e.g., infrared cameras or sensors).

The present disclosure includes a system, a method, and/or a computer program product. The computer program product may include a computer readable storage medium (or media) having computer readable program instructions thereon for causing a processor to carry out aspects of the present disclosure.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present disclosure may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider). In various embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present disclosure.

Aspects of the present disclosure are described herein with reference to flowchart illustrations and/or block diagrams of methods, apparatus (systems), and computer program products according to embodiments of the disclosure. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the figures illustrate the architecture, functionality, and operation of possible implementations of systems, methods, and computer program products according to various embodiments of the present disclosure. In this regard, each block in the flowchart or block diagrams may represent a module, segment, or portion of instructions, which comprises one or more executable instructions for implementing the specified logical function(s). In various alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

### APPENDIX

Lengthy table referenced here
US20220122696A1-20220421-T00001
Please refer to the end of the specification for access instructions.

Lengthy table referenced here
US20220122696A1-20220421-T00002
Please refer to the end of the specification for access instructions.

Lengthy table referenced here
US20220122696A1-20220421-T00003
Please refer to the end of the specification for access instructions.

Lengthy table referenced here
US20220122696A1-20220421-T00004
Please refer to the end of the specification for access instructions.

Lengthy table referenced here
US20220122696A1-20220421-T00005
Please refer to the end of the specification for access instructions.

Lengthy table referenced here
US20220122696A1-20220421-T00006
Please refer to the end of the specification for access instructions.

Lengthy table referenced here
US20220122696A1-20220421-T00007
Please refer to the end of the specification for access instructions.

Lengthy table referenced here
US20220122696A1-20220421-T00008
Please refer to the end of the specification for access instructions.

Lengthy table referenced here
US20220122696A1-20220421-T00009
Please refer to the end of the specification for access instructions.

Lengthy table referenced here
US20220122696A1-20220421-T00010
Please refer to the end of the specification for access instructions.

Lengthy table referenced here
US20220122696A1-20220421-T00011
Please refer to the end of the specification for access instructions.

Lengthy table referenced here
US20220122696A1-20220421-T00012
Please refer to the end of the specification for access instructions.

Lengthy table referenced here
US20220122696A1-20220421-T00013
Please refer to the end of the specification for access instructions.

Lengthy table referenced here
US20220122696A1-20220421-T00014
Please refer to the end of the specification for access instructions.

Lengthy table referenced here
US20220122696A1-20220421-T00015
Please refer to the end of the specification for access instructions.

Lengthy table referenced here
US20220122696A1-20220421-T00016
Please refer to the end of the specification for access instructions.

Lengthy table referenced here
US20220122696A1-20220421-T00017
Please refer to the end of the specification for access instructions.

Lengthy table referenced here
US20220122696A1-20220421-T00018
Please refer to the end of the specification for access instructions.

Lengthy table referenced here
US20220122696A1-20220421-T00019
Please refer to the end of the specification for access instructions.

Lengthy table referenced here
US20220122696A1-20220421-T00020
Please refer to the end of the specification for access instructions.

Lengthy table referenced here
US20220122696A1-20220421-T00021
Please refer to the end of the specification for access instructions.

Lengthy table referenced here
US20220122696A1-20220421-T00022
Please refer to the end of the specification for access instructions.

Lengthy table referenced here
US20220122696A1-20220421-T00023
Please refer to the end of the specification for access instructions.

Lengthy table referenced here
US20220122696A1-20220421-T00024
Please refer to the end of the specification for access instructions.

Lengthy table referenced here
US20220122696A1-20220421-T00025
Please refer to the end of the specification for access instructions.

Lengthy table referenced here
US20220122696A1-20220421-T00026
Please refer to the end of the specification for access instructions.

Lengthy table referenced here
US20220122696A1-20220421-T00027
Please refer to the end of the specification for access instructions.

Lengthy table referenced here
US20220122696A1-20220421-T00028
Please refer to the end of the specification for access instructions.

Lengthy table referenced here
US20220122696A1-20220421-T00029
Please refer to the end of the specification for access instructions.

LENGTHY TABLES
The patent application contains a lengthy table section. A copy of the table is available in electronic form from the USPTO web site (https://seqdata.uspto.gov/?pageRequest=docDetail&DocID=US20220122696A1). An electronic copy of the table will also be available from the USPTO upon request and payment of the fee set forth in 37 CFR 1.19(b)(3).

What is claimed is:

1. A method of generating an enhanced set of sequences for taxonomical classification, the method comprising:
receiving a plurality of reference sequences, wherein each of the plurality of reference sequences corresponds to a taxonomical classification;
assigning to each of a plurality of supplemental sequences a label corresponding to at least one of the reference sequences;
truncating each of the plurality of supplemental sequences and each of the plurality of reference sequences to a region of interest to thereby generate a truncated set of sequences;
measuring similarity between pairs of truncated sequences in the truncated set of sequences to determine whether the similarity is above a predetermined threshold; and
assigning an intermediate taxonomical label to the pair of truncated sequences in the truncated set of sequences when the similarity is above the predetermined threshold to thereby generate an enhanced set of sequences.

2. The method of claim 1, wherein the plurality of reference sequences comprises RNA.

3. The method of claim 1, wherein the plurality of reference sequences comprises DNA.

4. The method of claim 3, wherein the plurality of reference sequences comprises a genome.

5. The method of claim 1, wherein each taxonomical classification comprises a species.

6. The method of claim 1, wherein the plurality of reference sequences comprises sequences of microbial organisms.

7. The method of claim 1, wherein the plurality of supplemental sequences comprises sequences of microbial organisms.

8. The method of claim 1, wherein the plurality of reference sequences comprises sequences of eukaryotes.

9. The method of claim 1, wherein the plurality of supplemental sequences comprises sequences of eukaryotes.

10. The method of claim 1, wherein the plurality of supplemental sequences comprises sequences from a next generation sequencer.

11. The method of claim 1, wherein the supplemental data comprise data from published studies.

12. The method of claim 1, wherein the supplemental data comprise data from isolated colonies.

13. The method of claim 1, wherein the supplemental data comprise data from a clone library.

14. The method of claim 1, further comprising:
collecting microbial organisms from a predetermined site;
performing amplification of at least one sequence of the microbial organisms;
sequencing the at least one amplified sequence.

15. The method of claim 14, wherein the predetermined site is a part of a human body.

16. The method of claim 15, wherein the part of the human body is an aerodigestive tract.

17. The method of claim 14, wherein the supplemental data comprises the sequenced amplified sequence of the collected microbial organisms.

18. The method of claim 1, wherein the region of interest comprises a V1-V3 region.

19. The method of claim 1, further comprising removing duplicates in the truncated set of sequences.

20. The method of claim 1, wherein the assigned label comprises the respective taxonomical classification of each of the reference sequences.

21. The method of claim 1, wherein the intermediate taxonomical classification is hierarchically inferior to a genus.

22. The method of claim 21, wherein the intermediate taxonomical classification is hierarchically superior to a species.

23. The method of claim 1, wherein the intermediate taxonomical classification is between a genus and a species.

24. The method of claim 1, wherein the predetermined threshold is greater than or equal to 90%.

25. The method of claim 1, wherein the predetermined threshold is greater than or equal to 98.5%.

26. The method of claim 1, wherein the predetermined threshold is determined based on a breadth of taxonomical classification.

27. The method of claim 1, wherein measuring similarity comprises comparing nucleotides between pairs of truncated sequences in the truncated set of sequences.

28. The method of claim 1, further comprising training a machine learning classifier on the enhanced set of data.

29. The method of claim 28, wherein applying the trained machine learning classifier results in an error rate of less than or equal to 1.5%.

30. The method of claim 28, wherein applying the trained machine learning classifier results in an error rate of less than or equal to 0.5%.

31. The method of claim 28, wherein applying the trained machine learning classifier results in a no-call rate of less than or equal to 40%.

32. The method of claim 28, wherein applying the trained machine learning classifier results in a no-call rate of less than or equal to 10%.

33. A computer program product for generating an enhanced set of sequences for taxonomical classification, the computer program product comprising a computer readable storage medium having program instructions embodied therewith, the program instructions executable by a processor to cause the processor to perform a method comprising:
receiving a plurality of reference sequences, wherein each of the plurality of reference sequences corresponds to a taxonomical classification;
assigning to each of a plurality of supplemental sequences a label corresponding to at least one of the reference sequences;
truncating each of the plurality of supplemental sequences and each of the plurality of reference sequences to a region of interest to thereby generate a truncated set of sequences;
measuring similarity between pairs of truncated sequences in the truncated set of sequences to determine whether the similarity is above a predetermined threshold; and
assigning an intermediate taxonomical label to the pair of truncated sequences in the truncated set of sequences when the similarity is above the predetermined threshold to thereby generate an enhanced set of sequences.

34. The computer program product of claim 33, wherein the plurality of reference sequences comprises RNA.

35. The computer program product of claim 33, wherein the plurality of reference sequences comprises DNA.

36. The computer program product of claim 33, wherein the plurality of reference sequences comprises a genome.

37. The computer program product of claim 33, wherein each taxonomical classification comprises a species.

38. The computer program product of claim 33, wherein the plurality of reference sequences comprises sequences of a microbial organism.

39. The computer program product of claim 33, wherein the plurality of supplemental sequences comprise sequences of a microbial organism.

40. The computer program product of claim 33, wherein the plurality of reference sequences comprises sequences of eukaryotes.

41. The computer program product of claim 33, wherein the plurality of supplemental sequences comprises sequences of eukaryotes.

42. The computer program product of claim 33, wherein the plurality of supplemental sequences comprises sequences from a next generation sequencer.

43. The computer program product of claim 33, wherein the supplemental data comprise data from published studies.

44. The computer program product of claim 33, wherein the supplemental data comprise data from isolated colonies.

45. The computer program product of claim 33, wherein the supplemental data comprise data from a clone library.

46. The computer program product of claim 33, wherein the region of interest comprises a V1-V3 region.

47. The computer program product of claim 33, further comprising removing duplicate in the truncated set of sequences.

48. The computer program product of claim 33, wherein the assigned label comprises the respective taxonomical classification of each of the reference sequences.

49. The computer program product of claim 33, wherein the intermediate taxonomical classification is hierarchically inferior to a genus.

50. The computer program product of claim 49, wherein the intermediate taxonomical classification is hierarchically superior to a species.

51. The computer program product of claim 33, wherein the intermediate taxonomical classification is between a genus and a species.

52. The computer program product of claim 33, wherein the predetermined threshold is greater than or equal to 90%.

53. The computer program product of claim 33, wherein the predetermined threshold is greater than or equal to 98.5%.

54. The computer program product of claim 33, wherein the predetermined threshold is determined based on a breadth of taxonomical classification.

55. The computer program product of claim 33, wherein measuring similarity comprises comparing nucleotides between pairs of truncated sequences in the truncated set of sequences.

56. The computer program product of claim 33, further comprising training a machine learning classifier on the enhanced set of data.

57. The computer program product of claim 56, wherein applying the trained machine learning classifier results in an error rate of less than or equal to 1.5%.

58. The computer program product of claim 56, wherein applying the trained machine learning classifier results in an error rate of less than or equal to 0.5%.

59. The computer program product of claim 56, wherein applying the trained machine learning classifier results in a no-call rate of less than or equal to 40%.

60. The computer program product of claim 56, wherein applying the trained machine learning classifier results in a no-call rate of less than or equal to 10%.

61. A method for generating a species-labelled set of sequences, the method comprising:
isolating nucleic acids from a microbial source;
amplifying a predetermined region of the nucleic acids to generate a sequence library;
sequencing the sequence library to generate a plurality of sequences; and
determining a species for each of the plurality of sequences to thereby generate a species-labelled set of sequences.

62. The method of claim 61, wherein determining a species for each of the plurality of sequences comprises:
receiving a plurality of reference sequences, wherein each of the plurality of reference sequences corresponds to a taxonomical classification;
assigning to each of a plurality of supplemental sequences a label corresponding to at least one of the reference sequences;
truncating each of the plurality of supplemental sequences and each of the plurality of reference sequences to a region of interest to thereby generate a truncated set of sequences;
measuring similarity between pairs of truncated sequences in the truncated set of sequences to determine whether the similarity is above a predetermined threshold; and
assigning an intermediate taxonomical label to the pair of truncated sequences in the truncated set of sequences when the similarity is above the predetermined threshold to thereby generate an enhanced set of sequences.

63. The method of claim 61 or 62, wherein the predetermined region is a 16S rRNA region.

64. The method of claim 63, wherein the 16S rRNA region is a V1-V3 region.

65. The method of any one of claims 61-64, wherein sequencing is performed without overlapping reads.

66. The method of any one of claims 61-65, wherein amplification comprises applying a one or more primers to the nucleic acids.

67. The method of any one of claims 61-66, wherein amplification is performed for a predetermined number of cycles.

68. The method of any one of claims 61-67, wherein sequencing comprises sequencing a reversed V3 region first and then sequencing a V1 region.

69. The method of any one of claims 61-68, wherein amplification comprises applying an adaptor-ligated library.

70. The method of claim 69, wherein as the adaptor-ligated library comprises a PhiX genome.

71. The method of any one of claims 61-70, wherein sequencing comprises asymmetric sequencing.

72. The method of claim 71, wherein asymmetric sequencing comprises reading alternating sides of the nucleic acids.

73. The method of claim 71, wherein asymmetric sequencing comprises alternating read lengths of 100 base pairs and 400 base pairs.

74. The method of any one of claims 61-73, wherein determining a species for each of the plurality of sequences comprises applying a RDP algorithm to the plurality of sequences.


SEQUENCE LISTING (5 sequences)

SEQ ID NO: 1 - DNA, 66 bp, Artificial Sequence
  synthetic primer
aatgatacggcgaccaccgagatctacacatcgtacgtatggtaattcaattaccgcggctgctgg

SEQ ID NO: 2 - DNA, 63 bp, Artificial Sequence
  synthetic primer
caagcagaagacggcatacgagataactctcgagtcagtcagccgagtttgatcmtggctcag

SEQ ID NO: 3 - DNA, 29 bp, Artificial Sequence
  synthetic primer
tatggtaattcaattaccgcggctgctgg

SEQ ID NO: 4 - DNA, 31 bp, Artificial Sequence
  synthetic primer
agtcagtcagccgagtttgatcmtggctcag

SEQ ID NO: 5 - DNA, 31 bp, Artificial Sequence
  synthetic primer
ctgagccakgatcaaactcggctgactgact